(19) United States
(12) Patent Application Publication
(10) Pub. No.: US 2024/0002350 A1
(43) Pub. Date: Jan. 4, 2024

(54) PROTEIN AND LIPID THERAPEUTIC TARGETS

(71) Applicants: UNIVERSITY OF PITTSBURGH-OF THE COMMONWEALTH SYSTEM OF HIGHER EDUCATION, Pittsburgh, PA (US); NATIONAL CENTER FOR ADVANCING TRANSLATIONAL SCIENCES/DPI/NIH, Rockville, MD (US)

(72) Inventors: Sally Ellen MORGANROTH, Pittsburgh, PA (US); Valerian KAGAN, Pittsburgh, PA (US); Jinming ZHAO, Pittsburgh, PA (US); Ganesha RAI, Rockville, MD (US); Hulya BAYIR, Pittsburgh, PA (US); Yulia TYURINA, Pittsburgh, PA (US); Haider DAR, Pittsburgh, PA (US); Tamil ANTHONYMUTHU, Pittsburgh, PA (US); Joel GREENBERGER, Pittsburgh, PA (US); Michael EPPERLY, Pittsburgh, PA (US); Diane LUCI, Okatie, SC (US); Juan MARUGAN, Gaithersburg, MD (US); Anton SIMEONOV, Bethesda, MD (US); Alexey V. ZAKHAROV, Ijamsville, MD (US); Adam YASGAR, Bethesda, MD (US); Andrew AMOSCATO, Pittsburgh, PA (US)

(21) Appl. No.: 18/037,406

(22) PCT Filed: Nov. 17, 2021
(86) PCT No.: PCT/US2021/059641

§ 371 Date: May 17, 2023

Related U.S. Application Data
(60) Provisional application No. 63/114,760, filed on Nov. 17, 2020.

(51) Int. Cl. C07D 239/42; C07D 233/84; C07D 233/88; C07D 403/12; C07C 335/16; C07D 401/04; C07D 237/20; C07D 249/10; C07D 263/46; C07D 263/48; C07D 277/36; C07D 277/42; A61P 25/00

(52) U.S. Cl. CPC C07D 239/42; C07D 233/84; C07D 233/88; C07D 403/12; C07C 335/16; C07D 401/04; C07D 237/20; C07D 249/10; C07D 263/46; C07D 263/48; C07D 277/36; C07D 277/42; A61P 25/00

(57) ABSTRACT

Disclosed are compounds and methods for preventing or treating necroinflammation associated with ferroptotic processes. The method includes inhibiting 15 lipoxygenase/phosphatidylethanolamine binding protein (15LOX/PEBP1) complex, wherein the inhibitor exhibits a higher binding affinity or binding activity for 15LOX/PEBP1 complex compared to 15LOX alone. Necroinflammation associated with ferroptotic processes causes several pathogenic conditions including upper or lower respiratory disorders, acute or chronic brain injury, renal injury, injury by radiation, neurodegenerative disorder, among others. The disclosed compounds and methods are useful in subjects diagnosed with one or more of these conditions.

### CROSS-REFERENCE TO RELATED APPLICATIONS

This application claims the benefit of priority to U.S. Provisional Application No. 63/114,760 filed Nov. 17, 2020, which is hereby incorporated herein by reference in its entirety.

### STATEMENT REGARDING FEDERALLY SPONSORED RESEARCH

This invention was made with government support under grant numbers AI145406, AI106684, and AI040600 awarded by the National Institutes of Health. The government has certain rights in the invention.

### BACKGROUND

Harmonized epithelial cell communities depend on the fidelity of their individual members to eliminate metabolic and/or toxic consequences to individual cells that subsequently endanger the health of the entire population. Several cell death programs have been identified as instruments of this sacrificial behavior, beginning with apoptosis and more recently expanded into other contextually regulated necrotic platforms (e.g., necroptosis, pyroptosis). Ferroptosis, the response to redox disbalance between the prooxidant enzymatically driven reactions of lipid peroxidation and their thiol-dependent control by glutathione (GSH) peroxidases, is a recent addition to these programmed cell death pathways; 15-lipoxygenase (15LO)-, when interacting with a 2^{nd} protein, phosphatidylethanolamine binding protein-1 (PEBP1) initiates peroxidation of arachidonoyl-phosphatidylethanolamines (AA-PEs) to hydroperoxy-products which distinguishes the characteristic features of ferroptotic cell demise. Because of the irrevocability of cell death, a variety of protective processes can be triggered to prevent and/or eliminate the “damage” by activating repair mechanisms. Among them are the reduction of unstable hydroperoxy-lipids to the stable alcohol-derivatives, lowering the amounts of oxidizable polyunsaturated lipid substrates by suppressing the activity of participating enzymes, Acyl-CoA-Synthase Long Chain 4 (ACSL4) and Lysophospholipid Acyl Transferase 3 (LPCAT4), activation of Ferroptosis Suppressor Protein 1 (FSP1), activation of autophagy, among others.

There is a need for compounds, compositions, and methods that regulate the mechanisms related to ferroptosis. Particularly, there is a need for compounds, compositions, and methods that modulate the ferroptotic cell death program in various cell types. The compounds, compositions, and methods disclosed herein address these and other needs.

### SUMMARY

In accordance with the purposes of the disclosed materials and methods, as embodied and broadly described herein, the disclosed subject matter, in one aspect, relates to compounds, compositions and methods of using compounds and compositions. The compounds, compositions, and methods disclosed herein can be used for treating or preventing necroinflammation associated with ferroptotic processes promoting upper or lower respiratory disorders, acute or chronic brain injury, renal injury, injury by radiation, neurodegenerative disorder, or a combination thereof in a subject. The particular methods can include administering a therapeutically effective amount of a compound or pharmaceutical composition that inhibits 15 lipoxygenase/phosphatidylethanolamine binding protein (15LOX/PEBP1) complexes, wherein the compounds or pharmaceutical compositions exhibit a higher binding affinity or binding activity for 15LOX/PEBP1 complex compared to 15LOX alone. This can be important since 15-HOO-AA-PE (formed by 15LOX/PEBP1 complex) represents pro-ferroptotic signals, while oxygenated free HOO-AA does not. Further, it has been shown that complexation of 15LOX with PEBP1 resulted in a gain of a new catalytic competence toward oxygenating PUFA-PE. In some examples, the compounds or pharmaceutical compositions disclosed herein can exhibit at least 2, at least 5, at least 10, at least 20, or at least 50 times higher binding affinity or binding activity for 15LOX/PEBP1 complex compared to 15LOX alone. Preferably, the compounds or pharmaceutical compositions do not bind to 15LOX alone.

In further embodiments, the methods for treating or preventing necroinflammation associated with ferroptotic processes can include inhibiting interaction of 15 lipoxygenase (15LOX) with phosphatidylethanolamine binding protein PEBP1, thus inhibiting formation of (15LOX/PEBP1) complex in the subject. In other embodiments, the method can include inhibiting accumulation of 15-hydroperoxy-eicasotetraenoyl-phosphatidylethanolamines (15 HpETE-PE) in the subject. In other further embodiments, the method can include inhibiting oxidation of arachidonic acid containing phosphatidylethanolamines (AA-PE).

As described herein, necroinflammation associated with ferroptotic processes promotes certain conditions including, but not limited to, upper or lower respiratory disorders, acute or chronic brain injury, renal injury, injury by radiation, neurodegenerative disorder, or a combination thereof. In some instances of the disclosed methods, the subject can be diagnosed with an upper or lower respiratory disorders such as asthma, COPD, bronchitis, cystic fibrosis, nasal polyps, sinusitis, or a combination thereof. When the respiratory disorder is asthma, the subject can be diagnosed with exacerbation prone asthma or other characteristics. In other instances of the disclosed methods, the subject can be diagnosed with a kidney injury, such as renal failure. In further instances of the disclosed methods, the subject can be diagnosed with an acute traumatic brain injury or a chronic traumatic brain injury. The methods disclosed can suppress TBI-induced neuronal death, preserve or restore at least a portion of motor function, sensory function, cognitive function, visual function, auditory function, or a combination thereof in the subject. The subject can also be diagnosed with injury caused by total body gamma-irradiation. In some instances, the subject can be diagnosed with a neurodegenerative disorder. Depending on the specific condition of the subject, the methods disclosed herein can further comprise administering an additional therapeutically active co-agent used in the treatment of upper or lower respiratory disorders, acute or chronic brain injury, renal injury, injury by radiation, neurodegenerative disorder, or a combination thereof in a subject.

The specific compounds disclosed herein can have a structure according to Formula I:

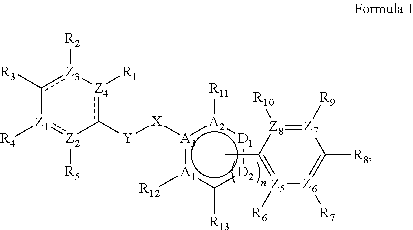

wherein
A₁, A₂, and A₃ are independently selected from C, N or S;
D₁ and D₂ are independently selected from O, CR′, NR′ or S wherein R′ is absent or selected from hydrogen, C₁-C₆ alkyl, C₂-C₆ alkenyl, C₂-C₆ alkynyl, aryl, alkylaryl, heteroaryl, or alkylheteroaryl, wherein R′ is optionally substituted or unsubstituted;
X is absent or selected from O, S, SO, SO₂, CHR′, or NR′, wherein R′ is selected from hydrogen, alkyl, or cycloalkyl;
Y is a bond, —C₁-C₃ alkyl, —C₂-C₃ alkenyl, —C₁-C₃ haloalkyl, —C₂-C₃ haloalkenyl, —C₁-C₃ alkyloxy, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, —C₁-C₃ ester, or cyclic, and wherein Y is optionally substituted with one or more groups;
Z₁, Z₂, Z₃, Z₄, Z₅, Z₆, Z₇, and Z₈ are independently selected from C, CH, O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, cyano, C₁-C₆ alkyl, C₂-C₆ alkenyl, C₂-C₆ alkynyl, aryl, alkylaryl, heteroaryl, or alkylheteroaryl, or R₁₁ can combine with D₁ to form a six membered ring, wherein R₁₁ is optionally substituted or unsubstituted;
R₁₂ is absent or selected from hydrogen, cyano, C₁-C₆ alkyl, C₂-C₆ alkenyl, C₂-C₆ alkynyl, aryl, alkylaryl, heteroaryl, or alkylheteroaryl, or R₁₂ can combine with X to form a six membered ring, wherein R₁₂ is optionally substituted or unsubstituted;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, or R₁₃ can combine with D₂ to form a six membered ring, wherein R₁₃ is optionally substituted or unsubstituted;
------ represents a bond that is present or absent;
n is 0 or 1;
or a pharmaceutically acceptable salt, ester, or prodrug thereof.

In some embodiments of the compounds, X is selected from S, O or NH and Y is —C₁-C₃ alkyl. In further embodiments, R₃ is selected from hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfoxide-, —C₁-C₆ alkylsulfonyl-, —C₁-C₆ alkylsulfonamide-, cyano, amide, alkylamide, carbamate, or alkylcarbamate, wherein R₃ is optionally substituted or unsubstituted. In even further embodiments, R₁₁ and R₁₃ are selected from hydrogen, aryl, alkylaryl, heteroaryl, or alkylheteroaryl, wherein R₁₃ is optionally substituted or unsubstituted. In some embodiments, R₁₂ is selected from C₂-C₆ alkenyl, C₂-C₆ alkynyl, aryl, alkylaryl, heteroaryl, or alkylheteroaryl, wherein R₁₂ is optionally substituted or unsubstituted.

Pharmaceutical compositions comprising a therapeutic effective amount of the compounds disclosed herein and a pharmaceutically acceptable carrier are also described.

### DESCRIPTION OF THE FIGURES

FIGS. 1A-1D show that controlled cortical impact (CCI) results in increased 15LOX levels and decreased GPX4 levels (FIG. 1A) and GPX4 activity in ipsilateral cortex of injured vs. naïve PND17 rats (FIG. 1B). FIG. 1C shows object based co-localization of PEBP1 and 15LOX in brain tissue. Stitched image shows high resolution large area confocal scanning of 3×5 image fields. Left panels: overlaid emissions for immunolocalization of PEBP1 (red), 15LOX (green) and nuclei (blue). Right panels: co-localization analysis for 15LOX and PEBP1, with the number of spots having both proteins appearing yellow. Scale bar is 200 microns. FIG. 1D shows number of co-localized 15LOX2 and PEBP1 in PND17 brain tissue is increased at 4 h after CCI vs sham (mean±SD, *p<0.001 vs. naïve or sham, N=3-5/group).

FIG. 2 is a volcano plot showing increase in oxidized PE with +1 to +4 oxygens in pericontusional cortex after CCI vs sham (n=4/gr). Additional panels show identification of pro-ferroptotic PEox in rat brain cortex after CCI using high resolution Orbitrap Fusion Lumos Tribrid MS. The plot labeled 1) shows full mass spectrum of PE (rat brain after CCI). Inset: spectrum in the range of m/z from 782.1 to 782.9. Molecular ion with m/z 782.5350 (Peox) is shown in red. The plots labeled 2) and 3) show MS2 spectra of precursor ions with m/z 750.5451 and 766.5411 containing AA and corresponding to PE-O-18:1/20:4 and PE-18:0/20:4, respectively. The plot labeled 4) shows MS2 spectra of Peox with m/z 782.5350. Note that 2 species with 1 and 2 oxygens formed after oxidation of PE(18:1/20:4) and PE(18:0/20:4), respectively. The plots labeled 5) and 6) show fragmentation patterns of ions with m/z 319 and m/z 317 (335-H2O) generated by MS3 analysis of Peox with m/z 782.5350. The fragment with m/z 113 is diagnostic of the OH- and OOH-groups at 15^{th} carbon of AA (i.e. 15-OH-AA and 15-OOH-AA).

FIGS. 3A-3C show data for ACSL4 expression after traumatic brain injury (TBI) and Triacsin C neuroprotection in TBI: FIG. 3A shows CCI leads to increased ACSL4 expression in cortex vs naïve at 4 and 24 h after injury. Mean+SD, N=4/group, *p<0.05 vs 0 h. FIG. 3B shows neurons were subjected to severe mechanical stretch injury (3-4 psi), resulting in increased cell death that was rescued with Fer-1 (0.4 μM), ACSL4 inhibitor Triacsin C (10 μM), 15LOX inhibitor baicalein (5 μM). Mean±SD, N=3-4/group, *p<0.05 vs control, #p<0.05 vs vehicle. FIG. 3C shows Triacsin C (i.c.v) administration after CCI attenuated neurodegeneration assessed by Fluoroade B (FJB) positivity 24 h post-CCI. Mean+SD, N=4/group, *p<0.05 vs vehicle.

FIGS. 4A-4C show OOH-AA-PE (red) is a better substrate for iPLA2β than AA-PE (blue; FIG. 4A). Hydrolysis of AA and 15-OOH-AA by iPLA2β is shown. Insert: Differences (A) in AA and 15-OOH-AA level formed by iPLA2β. Deficiency of iPLA2β induced by doxycycline-inducible Cas9 plasmid (FIG. 4B) or shPNPLA9 (FIG. 4C) lead to increased HOO-AA-PE generation and ferroptosis. Means±SD, *P<0.05, ***P<0.001, n=3, two-way ANOVA with Sidak post-hoc test.

FIGS. 5A-5B show baicalein post-treatment reduces -PE oxidation after CCI (FIG. 5A). Volcano plots show speciation of PE oxidation in cortex of vehicle-treated vs naïve and baicalein-treated animals at 4 h post-CCI. FIG. 5B shows identities of the significantly elevated Peox species confirmed as proferroptotic 15-OOH-AA-PE with fragmentation analyses.

FIGS. 6A-6C show baicalein treatment after CCI. FIG. 6A shows decreased TUNEL positivity with baicalein vs vehicle after CCI. (Mean±SD, n=4-5/grp, *p<0.05 vs sham, #p<0.05 vs vehicle). FIG. 6B shows representative fluorescent images of DAPI and TUNEL staining in ipsilateral dentate gyrus (DG) and CA3 at 24 h after sham or CCI with baicalein or vehicle treatment. FIG. 6C shows vehicle-CCI group had significantly longer time to hidden platform on d10-14 post-CCI than sham and baicalein-CCI groups. There was no difference in time to reach visible platform (v) on d15 among groups, indicating a lack of motor or visual deficit. (Mean±SEM, n=10/grp, *p<0.05 vs sham and baicalein-CCI.)

FIGS. 7A-7C show 15LOX/PABP1 complex inhibitor, NCGC00599973-01. FIG. 7A shows ferroptosis induced by RSL3 or erastin; FIG. 7B shows RSL3 induced accumulation of PE-AA-OOH (mean±s.d. N=3), and FIG. 7C shows improved survival after whole body irradiation (9.25Gy) N=10/grp.

FIG. 8 shows binding of NCGC00599973-01 (white arrow) to 15LOX in the absence (left) or presence (right) of PEBP1. NCGC00599973-01 does not bind to 15LOX catalytic site (yellow circle) without PEBP1.

FIG. 9 shows cells treated with GPX4 inhibitor RSL3 (1.0 μM) in the presence of new LOX-PEBP1 complex inhibitors (500 nM concentration). FER.1 (500 nM) is positive control. Insert: Chemical formula of two of the compounds.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an injury” includes two or more such injuries, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., necroinflammation or ferroptosis). It is understood that this is typically in relation to some standard or expected value, in other words it is relative, but that it is not always necessary for the standard or relative value to be referred to. For example, “reduces necroinflammation” means decreasing the amount of inflammatory markers relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as exacerbation prone asthma), diminishment of extent of exacerbation prone asthma, stabilized (i.e., not worsening) state of exacerbation prone asthma, preventing or delaying exacerbation of the asthma, delaying occurrence or recurrence of asthma, delay or slowing of asthma progression, amelioration of the asthma state (including general symptoms), and remission (whether partial or total).

The term “patient” preferably refers to a human in need of treatment for any purpose, and more preferably a human in need of such a treatment to a respiratory disorder, a traumatic brain injury, a neurodegenerative disorder, a renal injury, or a combination thereof. However, the term “patient” can also refer to non-human animals, preferably mammals such as dogs, cats, horses, cows, pigs, sheep and non-human primates, among others, that are in need of treatment.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbols Rⁿ, Xⁿ, or Lⁿ is used herein as merely a generic substituent in the definitions below.

The term “alkoxy” as used herein is an alkyl group bound through a single, terminal ether linkage; that is, an “alkoxy” group can be defined as -OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. The cycloalkyl group can include 3 or more carbon atoms, such as 3, 4, 5, 6, 7, 8, 9, or 10 carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. The cycloalkyl group can include 3 or more carbon atoms, such as 3, 4, 5, 6, 7, 8, 9, or 10 carbon atoms. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O—.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to necroinflammation, an effective amount comprises an amount sufficient to (i) reduce ferroptotic processes; (ii) inhibit, retard, slow to some extent and preferably stop ferroptotic processes; (iii) prevent or delay occurrence and/or recurrence of ferroptotic processes; and/or (iv) relieve to some extent one or more of the symptoms associated with ferroptotic processes. An effective amount can be administered in one or more doses.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

Compounds

Disclosed herein are compounds that prevent ferroptosis by inhibiting 15 lipoxygenase/phosphatidylethanol-amine binding protein (15LOX/PEBP1) complex. In general, the compounds exhibit a higher binding affinity or binding activity 15LOX/PEBP1 complex compared to 15LOX (15LO1 or 15LO2) alone. For example, the compounds can exhibit at least 2 (e.g., at least 5, at least 10, at least 15, at least 20, at least 25, at least 30, at least 35, at least 40, at least 45, or at least 50) times higher binding affinity or binding activity for 15LOX/PEBP1 complex compared to 15LOX alone.

The compounds disclosed herein can have a structure according to Formula I below:

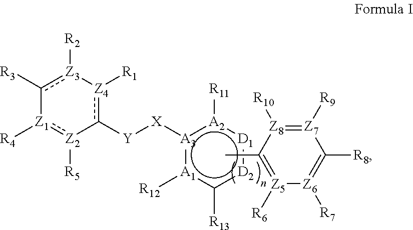

wherein
A₁, A₂, and A₃ are independently selected from C, N or S;
D₁ and D₂ are independently selected from O, CR′, NR′ or S wherein R′ is absent or selected from hydrogen, C₁-C₆ alkyl, C₂-C₆ alkenyl, C₂-C₆ alkynyl, aryl, alkylaryl, heteroaryl, or alkylheteroaryl, wherein R′ is optionally substituted or unsubstituted;
X is absent or selected from O, S, SO, SO₂, CHR′, or NR′, wherein R′ is selected from hydrogen, alkyl, or cycloalkyl;
Y is a bond, —C₁-C₃ alkyl-, —C₂-C₃ alkenyl-, —C₁-C₃ haloalkyl-, —C₂-C₃ haloalkenyl-, —C₁-C₃ alkyloxy-, —C₁-C₃ alkylamine-, —C₁-C₃ alkylamide-, —C₁-C₃ alkylsulfide-, —C₁-C₃ alkylthiol-, —C₁-C₃ alkylsulfoxide-, —C₁-C₃ alkylsulfonyl-, —C₁-C₃ alkylsulfonamide-, —C₁-C₃ ester-, or cyclic, and wherein Y is optionally substituted with one or more groups;
Z₁, Z₂, Z₃, Z₄, Z₅, Z₆, Z₇, and Z₈ are independently selected from C, CH, O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, cyano, C₁-C₆ alkyl, C₂-C₆ alkenyl, C₂-C₆ alkynyl, aryl, alkylaryl, heteroaryl, or alkylheteroaryl, or R₁ can combine with D₁ to form a six membered ring, wherein R₁₁ is optionally substituted or unsubstituted;
R₁₂ is absent or selected from hydrogen, cyano, C₁-C₆ alkyl, C₂-C₆ alkenyl, C₂-C₆ alkynyl, aryl, alkylaryl, heteroaryl, or alkylheteroaryl, or R₁₂ can combine with X to form a six membered ring, wherein R₁₂ is optionally substituted or unsubstituted;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, or R₁₃ can combine with D₂ to form a six membered ring, wherein R₁₃ is optionally substituted or unsubstituted;
[symbol] represents a bond that is present or absent;
n is 0 or 1;
or a pharmaceutically acceptable salt, ester, or prodrug thereof.

In certain embodiments, the compounds of Formula I can have a structure according to Formula I-A:

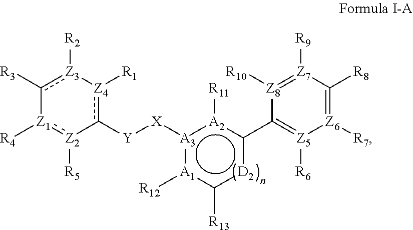

wherein
A₁, A₂, and A₃ are independently selected from C, N or S;
D₂ is selected from O, CR′, NR′ or S wherein R′ is absent or selected from hydrogen, or C₁-C₆ alkyl;
X is absent or selected from O, S, SO, SO₂, CHR′, or NR′, wherein R′ is selected from hydrogen, alkyl, or cycloalkyl;
Y is a bond, —C₁-C₃ alkyl-, —C₂-C₃ alkenyl-, —C₁-C₃ haloalkyl-, —C₂-C₃ haloalkenyl-, —C₁-C₃ alkyloxy-, —C₁-C₃ alkylamine-, —C₁-C₃ alkylamide-, —C₁-C₃ alkylsulfide-, —C₁-C₃ alkylthiol-, —C₁-C₃ alkylsulfoxide-, —C₁-C₃ alkylsulfonyl-, —C₁-C₃ alkylsulfonamide-, —C₁-C₃ ester-, or cyclic, and wherein Y is optionally substituted with one or more groups;
Z₁, Z₂, Z₃, Z₄, Z₅, Z₆, Z₇, and Z₈ are independently selected from C, CH, O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, cyano, C₁-C₆ alkyl, C₂-C₆ alkenyl, C₂-C₆ alkynyl, aryl, alkylaryl, heteroaryl, or alkylheteroaryl, wherein R₁₁ is optionally substituted or unsubstituted;
R₁₂ is absent or selected from hydrogen, cyano, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted; [symbol]
[symbol] represents a bond that is present or absent;
n is 0 or 1;
or a pharmaceutically acceptable salt, ester, or prodrug thereof.

In certain embodiments, the compounds of Formula I can have a structure according to Formula I-A:

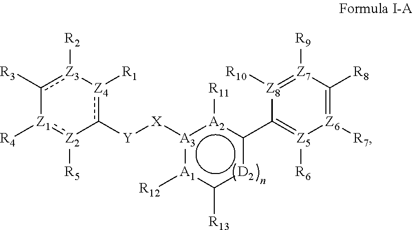

wherein
A₁, A₂, and A₃ are independently selected from C, N or S;
D₂ is selected from O, CR′, NR′ or S wherein R′ is absent or selected from hydrogen, or C₁-C₆ alkyl;
X is absent or selected from O, S, SO, SO₂, or NR′, wherein R′ is selected from hydrogen or alkyl;
Y is a bond, —C₁-C₃ alkyl-, —C₂-C₃ alkenyl-, —C₁-C₃ haloalkyl-, —C₂-C₃ haloalkenyl-, —C₁-C₃ alkyloxy-, —C₁-C₃ alkylamine-, —C₁-C₃ alkylamide-, —C₁-C₃ alkylsulfide-, —C₁-C₃ alkylthiol-, —C₁-C₃ alkylsulfoxide-, —C₁-C₃ alkylsulfonyl-, —C₁-C₃ alkylsulfonamide-, or —C₁-C₃ ester-, and wherein Y is optionally substituted with one or more groups;
Z₁, Z₂, Z₃, Z₄, Z₅, Z₆, Z₇, and Z₈ are independently selected from C, CH, O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, cyano, C₁-C₆ alkyl, C₂-C₆ alkenyl, C₂-C₆ alkynyl, aryl, alkylaryl, heteroaryl, or alkylheteroaryl, wherein R₁₁ is optionally substituted or unsubstituted;
R₁₂ is absent or selected from hydrogen, cyano, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is selected from hydrogen, C₁-C₆ alkyl, C₂-C₆ alkenyl, C₂-C₆ alkynyl, aryl, alkylaryl, heteroaryl, or alkylheteroaryl, wherein R₁₃ is optionally substituted or unsubstituted;
[symbol] represents a bond that is present or absent;
n is 0 or 1;
or a pharmaceutically acceptable salt, ester, or prodrug thereof.

In certain embodiments, the compounds of Formula I can have a structure according to Formula I-A′:

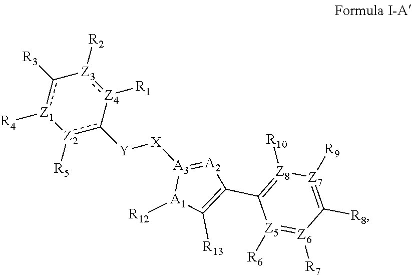

wherein
A₁, A₂, and A₃ are independently selected from C, N or S;
X is absent or selected from O, S, SO, SO₂, CHR′, or NR′, wherein R′ is selected from hydrogen alkyl, or cycloalkyl;
Y is a bond, —C₁-C₃ alkyl, —C₂-C₃ alkenyl, —C₁-C₃ haloalkyl, —C₂-C₃ haloalkenyl, —C₁-C₃ alkyloxy-, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, —C₁-C₃ ester, or cyclic, and wherein Y is optionally substituted with one or more groups;
Z₁, Z₂, Z₃, Z₄, Z₅, Z₆, Z₇, and Z₈ are independently selected from C, CH, or a heteroatom (e.g., O, S, or N);
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, C₁-C₆ alkyl, C₂-C₆ alkenyl, C₂-C₆ alkynyl, cycloalkyl, cycloalkenyl, heterocycloalkyl, cycloheteroalkenyl, aryl, alkylaryl, heteroaryl, or alkylheteroaryl, wherein R₁₁ is optionally substituted or unsubstituted;
R₁₂ is absent or selected from hydrogen, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted;
[symbol] represents a bond that is present or absent;
or a pharmaceutically acceptable salt, ester, or prodrug thereof.

In certain embodiments, the compounds of Formula I can have a structure according to Formula I-A′:

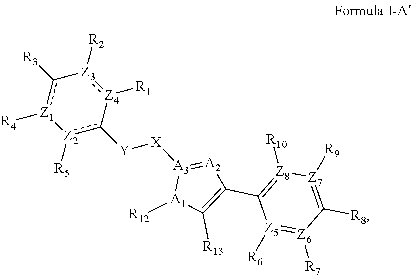

wherein
A₁, A₂, and A₃ are independently selected from C, N or S;
X is absent or selected from O, S, SO, SO₂, or NR′, wherein R′ is selected from hydrogen or alkyl;
Y is a bond, —C₁-C₃ alkyl, —C₂-C₃ alkenyl, —C₁-C₃ haloalkyl, —C₂-C₃ haloalkenyl, —C₁-C₃ alkyloxy-, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, or —C₁-C₃ ester, and wherein Y is optionally substituted with one or more groups;
Z₁, Z₂, Z₃, Z₄, Z₅, Z₆, Z₇, and Z₈ are independently selected from C, CH, or a heteroatom (e.g., O, S, or N);
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₁ is absent or selected from hydrogen, C₁-C₆ alkyl, C₂-C₆ alkenyl, C₂-C₆ alkynyl, cycloalkyl, cycloalkenyl, heterocycloalkyl, cycloheteroalkenyl, aryl, alkylaryl, heteroaryl, or alkylheteroaryl, wherein R₁₁ is optionally substituted or unsubstituted;
R₁₂ is absent or selected from hydrogen, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is selected from hydrogen, C₁-C₆ alkyl, C₂-C₆ alkenyl, C₂-C₆ alkynyl, aryl, alkylaryl, heteroaryl, or alkylheteroaryl, wherein R₁₃ is optionally substituted or unsubstituted;
[symbol] represents a bond that is present or absent;
or a pharmaceutically acceptable salt, ester, or prodrug thereof.

In certain embodiments, the compounds of Formula I can have a structure according to Formula I-A′-1:

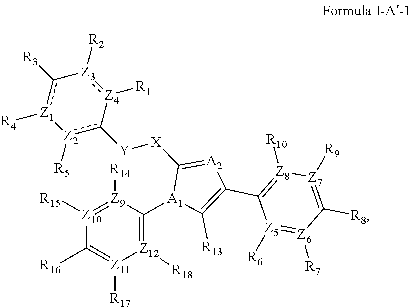

wherein
A₁ and A₂ are independently selected from C, N, or S;
X is absent or selected from O, S, SO, SO₂, CHR′, or NR′, wherein R′ is selected from hydrogen, alkyl, or cycloalkyl;
Y is a bond, —C₁-C₃ alkyl, —C₂-C₃ alkenyl, —C₁-C₃ haloalkyl, —C₂-C₃ haloalkenyl, —C₁-C₃ alkyloxy, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, —C₁-C₃ ester, or cyclic, and wherein Y is optionally substituted with one or more groups;
Z₁, Z₂, Z₃, Z₄, Z₅, Z₆, Z₇, Z₈, Z₉, Z₁₀, Z₁₁, and Z₁₂ are independently selected from C, CH, or a heteroatom (e.g., O, S, or N)O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁₄ to R₁₈ are optionally substituted with one or more groups;
[symbol] represents a bond that is present or absent;
or a pharmaceutically acceptable salt, ester, or prodrug thereof.

In certain embodiments, the compounds of Formula I can have a structure according to Formula I-A′-1:

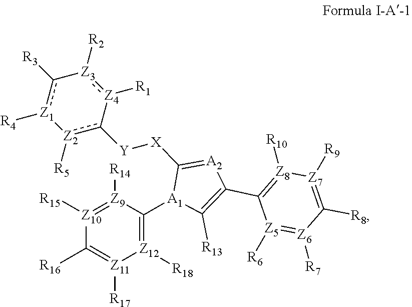

wherein
A₁ and A₂ are heteroatoms selected from N or S;
X is absent or selected from O, S, SO, SO₂, or NR′, wherein R′ is selected from hydrogen or alkyl;
Y is a bond, —C₁-C₃ alkyl, —C₂-C₃ alkenyl, —C₁-C₃ haloalkyl, —C₂-C₃ haloalkenyl, —C₁-C₃ alkyloxy, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, or —C₁-C₃ ester, and wherein Y is optionally substituted with one or more groups;
Z₁, Z₂, Z₃, Z₄, Z₅, Z₆, Z₇, Z₈, Z₉, Z₁₀, Z₁₁, and Z₁₂ are independently selected from C, CH, or a heteroatom (e.g., O, S, or N)O, S, or N;
R₁, R₂, R₄, and R₅ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently absent or selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₃ is selected from hydrogen, C₁-C₆ alkyl, C₂-C₆ alkenyl, C₂-C₆ alkynyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and
wherein R₁₄ to R₁₈ are optionally substituted with one or more groups;
[symbol] represents a bond that is present or absent;
or a pharmaceutically acceptable salt, ester, or prodrug thereof.

In some embodiments, the compounds of Formula I, Formula I-A, Formula I-A′, or Formula I-A′-1, can have a structure according to Formula I-A′-2:

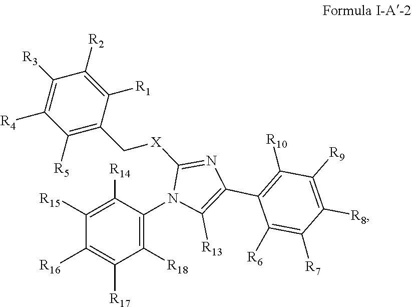

wherein
X is absent or selected from S, SO₂, CHR′, or NR′, wherein R′ is selected from hydrogen, alkyl, or cycloalkyl;
R₁, R₂, R₄, and R₅ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₃ is selected from hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁₄ to R₁₈ are optionally substituted with one or more groups;
or a pharmaceutically acceptable salt, ester, or prodrug thereof.

In some embodiments, the compounds of Formula I, Formula I-A, Formula I-A′, or Formula I-A′-1, can have a structure according to Formula I-A′-2:

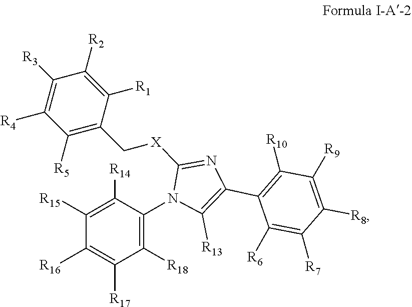

wherein
X is absent or selected from S, SO₂, or NR′, wherein R′ is selected from hydrogen or alkyl;
R₁, R₂, R₄, and R₅ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁, R₂, R₄, and R₅ are optionally substituted with one or more groups;
R₃ is selected from selected from hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₆ to R₁₀ are optionally substituted with one or more groups;
R₁₃ is selected from hydrogen, C₁-C₆ alkyl, C₂-C₆ alkenyl, C₂-C₆ alkynyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently selected from hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, alkylheteroaryl, and wherein R₁₄ to R₁₈ are optionally substituted with one or more groups;
or a pharmaceutically acceptable salt, ester, or prodrug thereof.

In some embodiments of Formula I, I-A, I-A′, I-A′-1, and I-A′-2, X can be selected from S or NH. For example, X can be S in some embodiments of Formula I, I-A, I-A′, I-A′-1, and I-A′-2. In other embodiments of Formula I, I-A, I-A′, I-A′-1, and I-A′-2, X can be NH.

In some embodiments of Formula I, I-A, I-A′, I-A′-1, and I-A′-2, Y can be selected from —C₁-C₃ alkyl, —C₁-C₃ alkoxy, or —C₁-C₃ alkylamide. For example, Y can be —C₁ alkyl, —C₂ alkyl, —C₃ alkyl, —C₁ alkoxy-, —C₂ alkoxy-, —C₃ alkoxy-, —C₁ alkylamide, —C₂ alkylamide, or —C₃ alkylamide, in some embodiments of Formula I, I-A, I-A′, I-A′-1, and I-A′-2. Y can be substituted or unsubstituted. For example, Y can be substituted with alkyl (for e.g., C₁-C₆ alkyl, C₁-C₃ alkyl, or methyl), halogen (for e.g., fluoro, chloro, or bromo), hydroxyl, or amine. In some instances, Y is unsubstituted. In some examples, Y can be —C₁-C₃ alkyl, —C₁-C₃ alkoxy-, or —C₁-C₃ alkylamide-, wherein Y can be optionally substituted with alkyl, halogen, hydroxyl, or amine. In further examples, Y can be —C₁-C₃ alkyl.

In some embodiments of Formula I, I-A, I-A′, I-A′-1, and I-A′-2, each of Z₁ to Z₁₂ are independently different. For example, Z₁ to Z₁₂ can be independently selected from C, CH, or a heteroatom (e.g., O, S, or N). In other embodiments of Formula I, I-A, I-A′, I-A′-1, and I-A′-2, Z₁ to Z₁₂ are all the same. For example, all of Z₁ to Z₁₂ can be C; or all of Z₁ to Z₁₂ can be CH; or all of Z₁ to Z₁₂ can be a heteroatom (e.g., O, S, or N). In some examples, Z₁ to Z₁₂ are all C.

In some examples of Formula I, I-A, I-A′, I-A′-1, and I-A′-2, all optional bonds are absent, such that the ring is a cycloalkyl group. In some examples of Formula I, I-A, I-A′, I-A′-1, and I-A′-2, all optional bonds are present, such that the ring is an aryl group.

In some examples of Formula I, I-A, I-A′, I-A′-1, and I-A′-2, at least one of A₁, A₂, and A₃ is not carbon.

In some embodiments of Formula I, I-A, I-A′, I-A′-1, and I-A′-2, A₁ and A₂ are independently different. For example, A₁ can be N and A₂ can be S. In other embodiments of Formula I, I-A, I-A′, I-A′-1, and I-A′-2, A₁ and A₂ are the same. For example, both A₁ and A₂ can be N or both A₁ and A₂ can be S. In some examples, A₁ and A₂ are the same and both are N.

In some embodiments of Formula I, I-A, I-A′, I-A′-1, and I-A′-2, R₁ is selected from hydrogen, halogen, alkyl, haloalkyl, haloalkenyl, alkoxy, haloalkoxy, amine, alkylamine, hydroxyl, cyano, or nitro. For example, R₁ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₁ can be substituted with one or more groups. For example, R₁ can be substituted with alkyl, halogen, hydroxyl, or amine. In some instances, however, R₁ is unsubstituted.

In some embodiments of Formula I, I-A, I-A′, I-A′-1, and I-A′-2, R₂ is selected from hydrogen, halogen, alkyl, haloalkyl, haloalkenyl, alkoxy, haloalkoxy, amine, alkylamine, hydroxyl, cyano, or nitro. For example, R₂ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₂ can be substituted with one or more groups. For example, R₂ can be substituted with alkyl, halogen, hydroxyl, or amine. In some instances, however, R₂ is unsubstituted.

In some embodiments of Formula I, I-A, I-A′, I-A′-1, and I-A′-2, R₃ is selected from hydrogen, halogen, alkyl, alkenyl, alkynyl, haloalkyl, haloalkenyl, haloalkynyl, alkoxy, haloalkoxy, amine, alkylamine, alkylsulfoxide, alkylsulfonyl, alkylsulfonamide, cyano, amide, alkylamide, carbamate, alkylcarbamate, hydroxyl, or nitro. For example, R₃ can be selected from hydrogen, C₁-C₆ alkyl (for e.g., methyl, ethyl, propyl, or butyl), C₂-C₆ alkenyl (for e.g., ethenyl, propenyl, or butenyl), C₂-C₆ alkynyl (for e.g., ethynyl, propynyl, or butynyl), C₁-C₆ haloalkyl (for e.g., trifluoromethyl, difluoromethyl), C₂-C₆ haloalkenyl, C₂-C₆ haloalkynyl, C₁-C₆ alkoxy (for e.g., methoxy, ethoxy, or propoxy), C₁-C₆ haloalkoxy, C₁-C₆ alkylamine (for e.g., methylamine or ethylamine), —C₁-C₆ alkylsulfoxide-, —C₁-C₆ alkylsulfonyl-, —C₁-C₆ alkylsulfonamide-, cyano, amide, alkylamide, carbamate, or alkylcarbamate. In some example, R₃ can be selected from hydrogen, halogen, C₁-C₆haloalkyl, C₂-C₆haloalkenyl, or C₁-C₆ haloalkoxy. In some embodiments, R₃ is not hydrogen. In other embodiments, R₃ is hydrogen. R₃ can be substituted with one or more groups. For example, R₃ can be substituted with alkyl, halogen, hydroxyl, or amine. In some instances, however, R₃ is unsubstituted.

In some embodiments of Formula I, I-A, I-A′, I-A′-1, and I-A′-2, R₄ is selected from hydrogen, halogen, alkyl, haloalkyl, haloalkenyl, alkoxy, haloalkoxy, amine, alkylamine, hydroxyl, cyano, or nitro. For example, R₄ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. In some example, R₄ is hydrogen. R₄ can be substituted with one or more groups. For example, R₄ can be substituted with alkyl, halogen, hydroxyl, or amine. In some instances, however, R₄ is unsubstituted.

In some embodiments of Formula I, I-A, I-A′, I-A′-1, and I-A′-2, R₅ is selected from hydrogen, halogen, alkyl, haloalkyl, haloalkenyl, alkoxy, haloalkoxy, amine, alkylamine, hydroxyl, cyano, or nitro. For example, R₅ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₅ can be substituted with one or more groups. For example, R₅ can be substituted with alkyl, halogen, hydroxyl, or amine. In some instances, however, R₅ is unsubstituted.

In some examples, R₁, R₂, and R₅ are independently selected from hydrogen, halogen, alkyl, haloalkyl, haloalkenyl, alkoxy, haloalkoxy, amine, alkylamine, hydroxyl, cyano, or nitro, wherein R₁, R₂, and R₅ are optionally substituted with one or more groups. In further examples, R₁, R₂, and R₅ are all hydrogen.

In some embodiments of Formula I, I-A, I-A′, I-A′-1, and I-A′-2, R₆ is selected from hydrogen, halogen, alkyl, haloalkyl, haloalkenyl, alkoxy, haloalkoxy, amine, alkylamine, hydroxyl, cyano, or nitro. For example, R₆ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₆ can be substituted with one or more groups. For example, R₆ can be substituted with alkyl, halogen, hydroxyl, or amine. In some instances, however, R₆ is unsubstituted.

In some embodiments of Formula I, I-A, I-A′, I-A′-1, and I-A′-2, R₇ is selected from hydrogen, halogen, alkyl, haloalkyl, haloalkenyl, alkoxy, haloalkoxy, amine, alkylamine, hydroxyl, cyano, or nitro. For example, R₇ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₇ can be substituted with one or more groups. For example, R₇ can be substituted with alkyl, halogen, hydroxyl, or amine. In some instances, however, R₇ is unsubstituted.

In some embodiments of Formula I, I-A, I-A′, I-A′-1, and I-A′-2, R₈ is selected from hydrogen, halogen, alkyl, alkenyl, alkynyl, haloalkyl, haloalkenyl, haloalkynyl, alkoxy, haloalkoxy, amine, alkylamine, alkylsulfoxide, alkylsulfonyl, alkylsulfonamide, cyano, amide, alkylamide, carbamate, alkylcarbamate, hydroxyl, or nitro. For example, R₈ can be selected from hydrogen, C₁-C₆ alkyl (for e.g., methyl, ethyl, propyl, or butyl), C₂-C₆ alkenyl (for e.g., ethenyl, propenyl, or butenyl), C₂-C₆ alkynyl (for e.g., ethynyl, propynyl, or butynyl), C₁-C₆ haloalkyl (for e.g., trifluoromethyl, difluoromethyl), C₂-C₆ haloalkenyl, C₂-C₆ haloalkynyl, C₁-C₆ alkoxy (for e.g., methoxy, ethoxy, or propoxy), C₁-C₆ haloalkoxy, C₁-C₆ alkylamine (for e.g., methylamine or ethylamine), —C₁-C₆ alkylsulfoxide-, —C₁-C₆ alkylsulfonyl-, —C₁-C₆ alkylsulfonamide-, cyano, amide, alkylamide, carbamate, or alkylcarbamate. In some example, R₈ is selected from hydrogen, halogen, C₁-C₆ haloalkyl, C₂-C₆ haloalkenyl, or C₁-C₆ haloalkoxy. In some embodiments, R₈ is not hydrogen. In other embodiments, R₈ can be hydrogen. R₈ can be substituted with one or more groups. For example, R₈ can be substituted with alkyl, halogen, hydroxyl, or amine. In some instances, however, R₈ is unsubstituted.

In some embodiments of Formula I, I-A, I-A′, I-A′-1, and I-A′-2, R₉ is selected from hydrogen, halogen, alkyl, haloalkyl, haloalkenyl, alkoxy, haloalkoxy, amine, alkylamine, hydroxyl, cyano, or nitro. For example, R₉ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. In some example, R₉ is hydrogen. R₉ can be substituted with one or more groups. For example, R₉ can be substituted with alkyl, halogen, hydroxyl, or amine. In some instances, however, R₉ is unsubstituted.

In some embodiments of Formula I, I-A, I-A′, I-A′-1, and I-A′-2, R₁₀ is selected from hydrogen, halogen, alkyl, haloalkyl, haloalkenyl, alkoxy, haloalkoxy, amine, alkylamine, hydroxyl, cyano, or nitro. For example, R₁₀ can be selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₁₀ can be substituted with one or more groups. For example, R₁₀ can be substituted with alkyl, halogen, hydroxyl, or amine. In some instances, however, R₁₀ is unsubstituted.

In some examples, R₆, R₇, and R₁₀ are independently selected from hydrogen, halogen, alkyl, haloalkyl, haloalkenyl, alkoxy, haloalkoxy, amine, alkylamine, hydroxyl, cyano, or nitro, wherein R₆, R₇, and R₁₀ are optionally substituted with one or more groups. In further examples, R₆, R₇, and R₁₀ are all hydrogen.

In some embodiments of Formula I, I-A, I-A′, I-A′-1, and I-A′-2, R₁₁ is selected from cycloalkyl, cycloalkenyl, heterocycloalkyl, heteroalkyl, cycloheteroalkenyl, aryl, or heteroaryl. For example, R₁₁ can be selected from aryl, alkylaryl, heteroaryl, or alkylheteroaryl. In some examples, R₁₁ can be selected from aryl or alkylaryl. In other examples, R₁₁ can be hydrogen. R₁₁ is optionally substituted with one or more groups. For example, R₁₁ can be substituted with one or more groups selected from halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, or —NR′R″, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkyl aryl, heteroaryl, or alkyl heteroaryl. In some instances, however, R₁₁ is unsubstituted.

In some embodiments of Formula I, I-A, I-A′, I-A′-1, and I-A′-2, R₁₂ is selected from C₂-C₆ alkenyl, C₂-C₆ alkynyl, cycloalkyl, cycloalkenyl, heterocycloalkyl, heteroalkyl, cycloheteroalkenyl, aryl, or heteroaryl. For example, R₁₂ can be selected from aryl, alkylaryl, heteroaryl, or alkylheteroaryl. In some examples, R₁₂ can be selected from aryl or alkylaryl. In some embodiments, R₁₂ is not hydrogen. R₁₂ is optionally substituted with one or more groups. For example, R₁₂ can be substituted with one or more groups selected from halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R′″, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, or —NR′R″, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkyl aryl, heteroaryl, or alkyl heteroaryl. In some instances, however, R₁₂ is unsubstituted.

In some embodiments of Formula I, I-A, I-A′, I-A′-1, and I-A′-2, R₁₃ is selected from cycloalkyl, cycloalkenyl, heterocycloalkyl, heteroalkyl, cycloheteroalkenyl, aryl, or heteroaryl. For example, R₁₃ can be selected from aryl, alkylaryl, heteroaryl, or alkylheteroaryl. In some examples, R₁₃ can be selected from aryl or alkylaryl. In other examples, R₁₃ can be hydrogen. R₁₃ is optionally substituted with one or more groups. For example, R₁₃ can be substituted with one or more groups selected from halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, or —NR′R″, wherein R′, R″, and R″′ are independently selected from hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkyl aryl, heteroaryl, or alkyl heteroaryl. For example, R₁₃ can be fluorinated alkyl or fluorinated cycloalkyl. In some instances, however, R₁₃ is unsubstituted.

In some embodiments of Formula I, I-A, I-A′, I-A′-1, and I-A′-2, R₁₄ is selected from hydrogen, halogen, alkyl, haloalkyl, haloalkenyl, alkoxy, haloalkoxy, amine, alkylamine, hydroxyl, cyano, or nitro. For example, R₁₄ is selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₁₄ can be substituted with one or more groups. For example, R₁₄ can be substituted with alkyl, halogen, hydroxyl, or amine. In some instances, however, R₁₄ is unsubstituted.

In some embodiments of Formula I, I-A, I-A′, I-A′-1, and I-A′-2, R₁₅ is selected from hydrogen, halogen, alkyl, alkenyl, alkynyl, haloalkyl, haloalkenyl, haloalkynyl, alkoxy, haloalkoxy, amine, alkylamine, alkylsulfoxide, alkylsulfonyl, alkylsulfonamide, cyano, amide, alkylamide, carbamate, alkylcarbamate, hydroxyl, or nitro. For example, R₁₅ can be selected from hydrogen, C₁-C₆ alkyl (for e.g., methyl, ethyl, propyl, or butyl), C₂-C₆ alkenyl (for e.g., ethenyl, propenyl, or butenyl), C₂-C₆ alkynyl (for e.g., ethynyl, propynyl, or butynyl), C₁-C₆ haloalkyl (for e.g., trifluoromethyl, difluoromethyl), C₂-C₆ haloalkenyl, C₂-C₆ haloalkynyl, C₁-C₆ alkoxy (for e.g., methoxy, ethoxy, or propoxy), C₁-C₆ haloalkoxy, C₁-C₆ alkylamine (for e.g., methylamine or ethylamine), —C₁-C₆ alkylsulfoxide-, —C₁-C₆ alkylsulfonyl-, —C₁-C₆ alkylsulfonamide-, cyano, amide, alkylamide, carbamate, or alkylcarbamate. In some example, R₁₅ is selected from hydrogen, halogen, C₁-C₆ haloalkyl, C₂-C₆ haloalkenyl, or C₁-C₆ haloalkoxy. In further examples, R₁₅ is not hydrogen. In other further examples, R₁₅ is hydrogen. R₁₅ can be substituted with one or more groups. For example, R₁₅ can be substituted with alkyl, halogen, hydroxyl, or amine. In some instances, however, R₁₅ is unsubstituted.

In some embodiments of Formula I, I-A, I-A′, I-A′-1, and I-A′-2, R₁₆ is selected from hydrogen, halogen, alkyl, haloalkyl, haloalkenyl, alkoxy, haloalkoxy, amine, alkylamine, hydroxyl, cyano, or nitro. For example, R₁₆ is selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. In some examples, R₁₆ is hydrogen. R₁₆ can be substituted with one or more groups. For example, R₁₆ can be substituted with alkyl, halogen, hydroxyl, or amine. In some instances, however, R₁₆ is unsubstituted.

In some embodiments of Formula I, I-A, I-A′, I-A′-1, and I-A′-2, R₁₇ is selected from hydrogen, halogen, alkyl, haloalkyl, haloalkenyl, alkoxy, haloalkoxy, amine, alkylamine, hydroxyl, cyano, or nitro. For example, R₁₇ is selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₁₇ can be substituted with one or more groups. For example, R₁₇ can be substituted with alkyl, halogen, hydroxyl, or amine. In some instances, however, R₁₇ is unsubstituted.

In some embodiments of Formula I, I-A, I-A′, I-A′-1, and I-A′-2, R₁₈ is selected from hydrogen, halogen, alkyl, haloalkyl, haloalkenyl, alkoxy, haloalkoxy, amine, alkylamine, hydroxyl, cyano, or nitro. For example, R₁₈ is selected from hydrogen, halogen (for e.g., bromo, chloro, or fluoro), alkyl (for e.g., methyl, ethyl, propyl, or butyl), haloalkyl (for e.g., trifluoromethyl, difluoromethyl), alkoxy (for e.g., methoxy, ethoxy, or propoxy), amine, alkylamine (for e.g., methylamine or ethylamine), or hydroxyl. R₁₈ can be substituted with one or more groups. For example, R₁₈ can be substituted with alkyl, halogen, hydroxyl, or amine. In some instances, however, R₁₈ is unsubstituted.

In some embodiments, R₁₄, R₁₇, and R₁₈ can be independently selected from hydrogen, halogen, alkyl, haloalkyl, haloalkenyl, alkoxy, haloalkoxy, amine, alkylamine, hydroxyl, cyano, or nitro, and wherein R₁₄, R₁₇, and R₁₈ are optionally substituted with one or more groups. In some examples, R₁₄, R₁₇, and R₁₈ are all hydrogen.

The compounds disclosed herein can have a structure below:

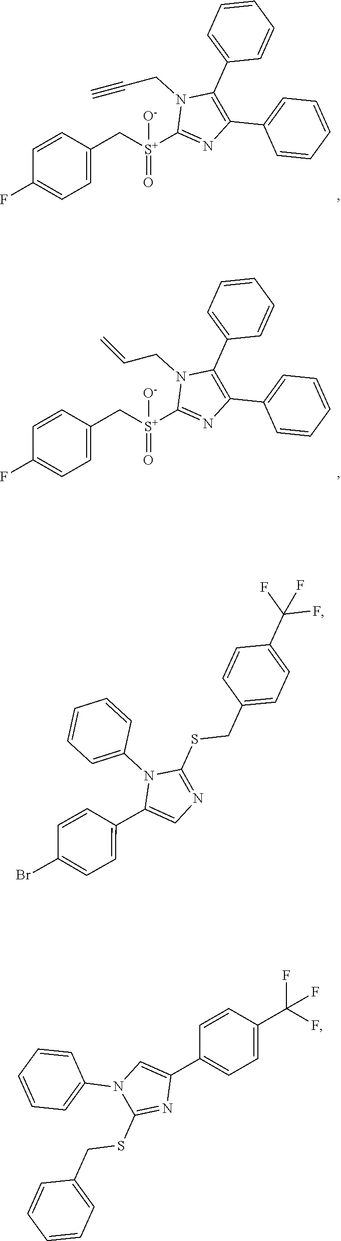

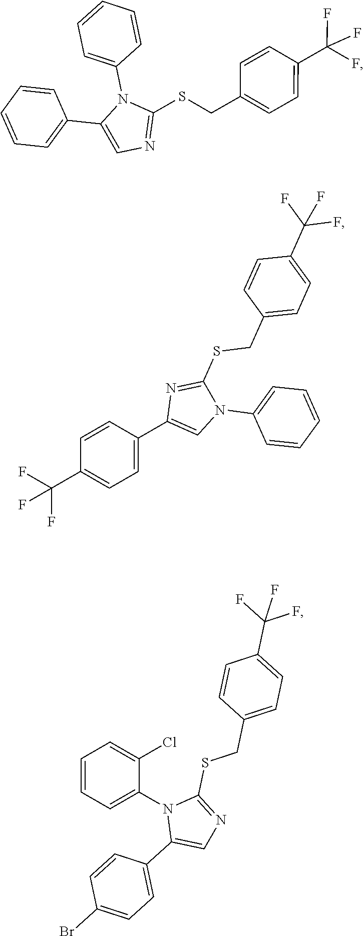

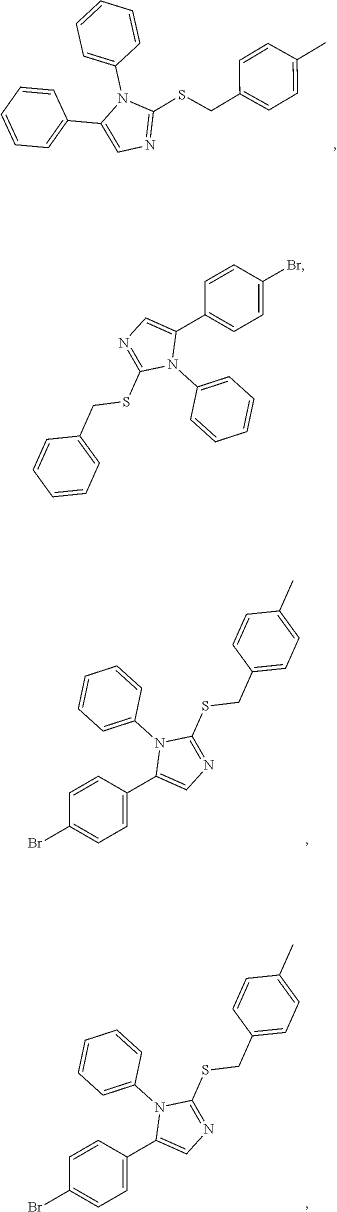

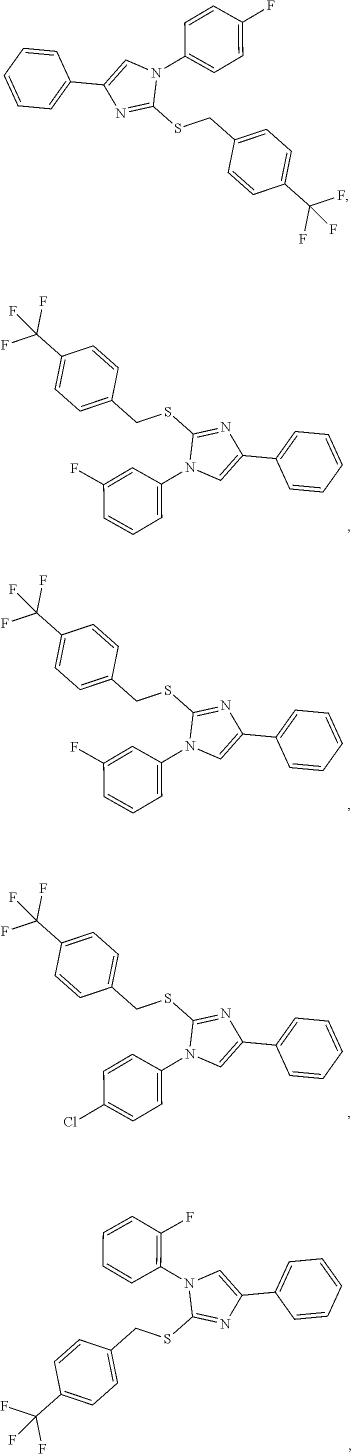

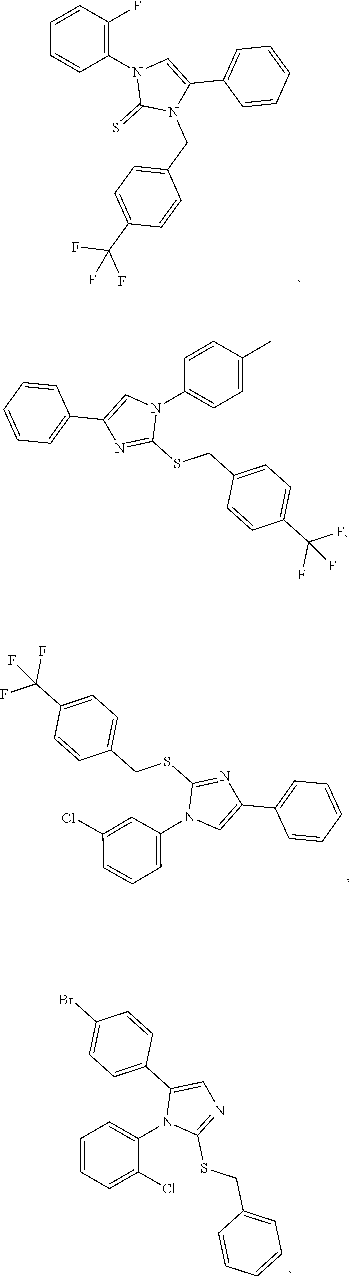

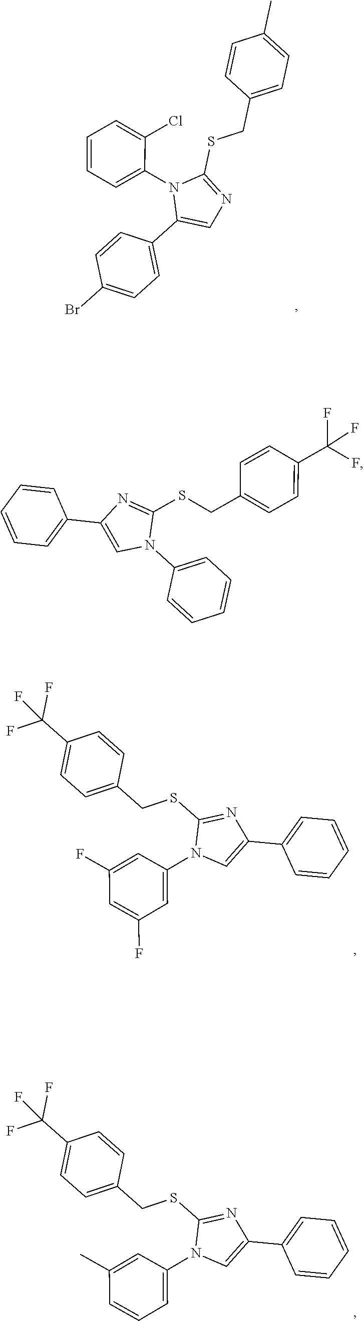

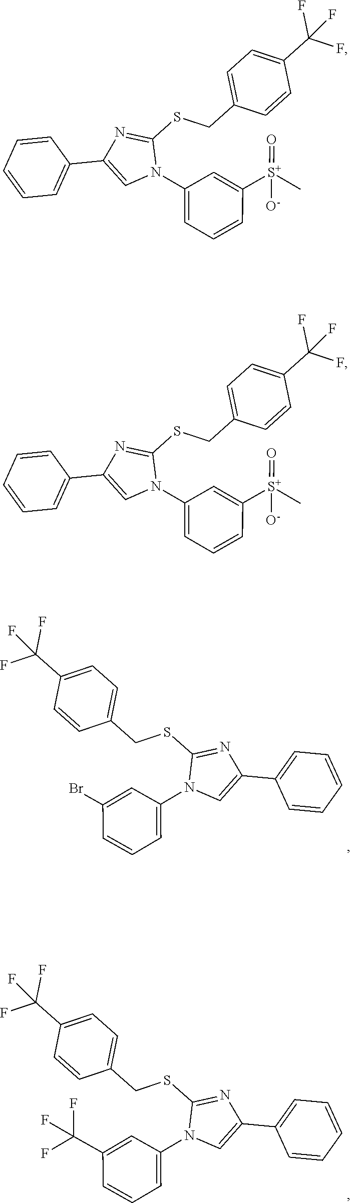

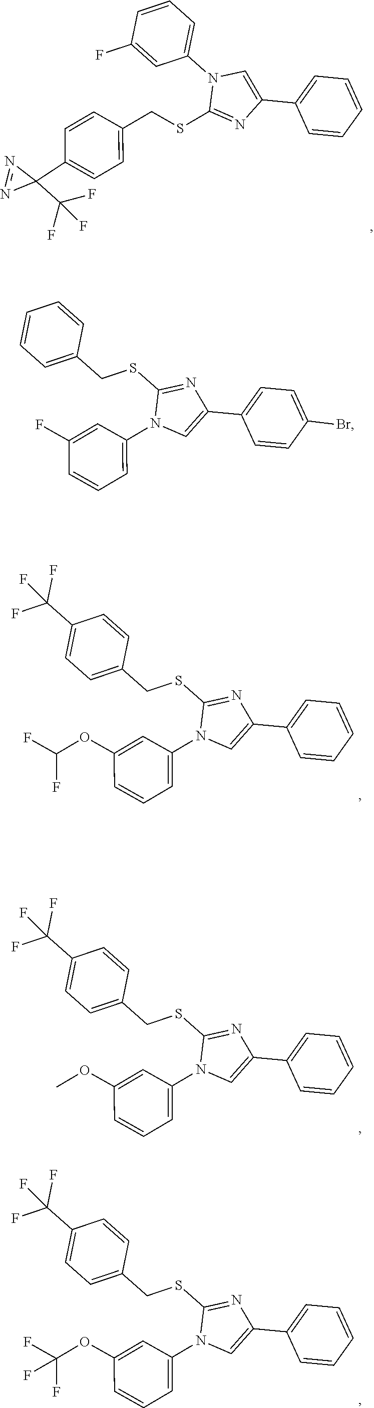

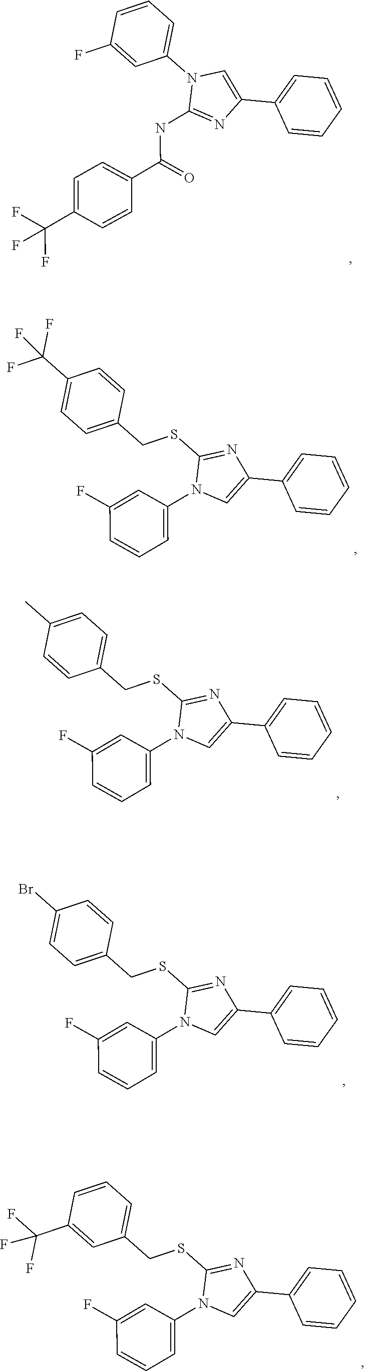

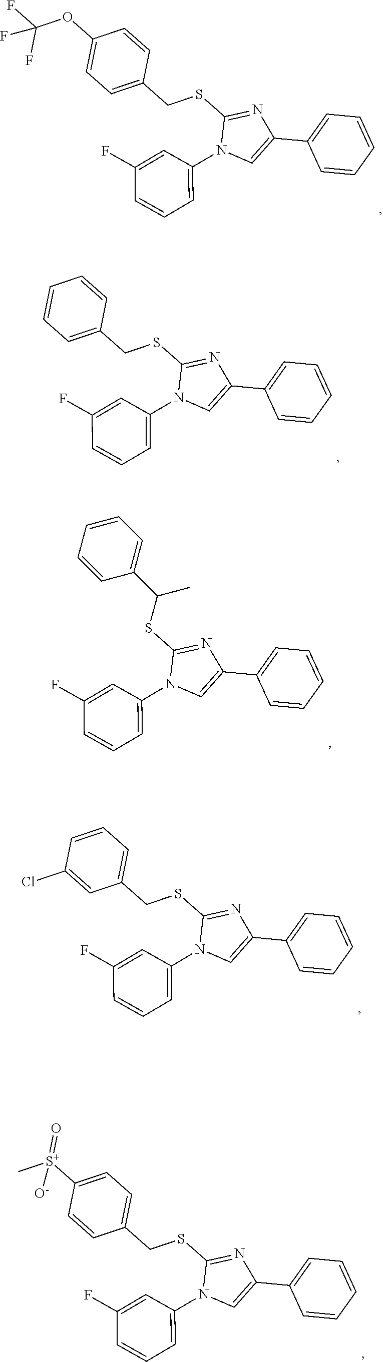

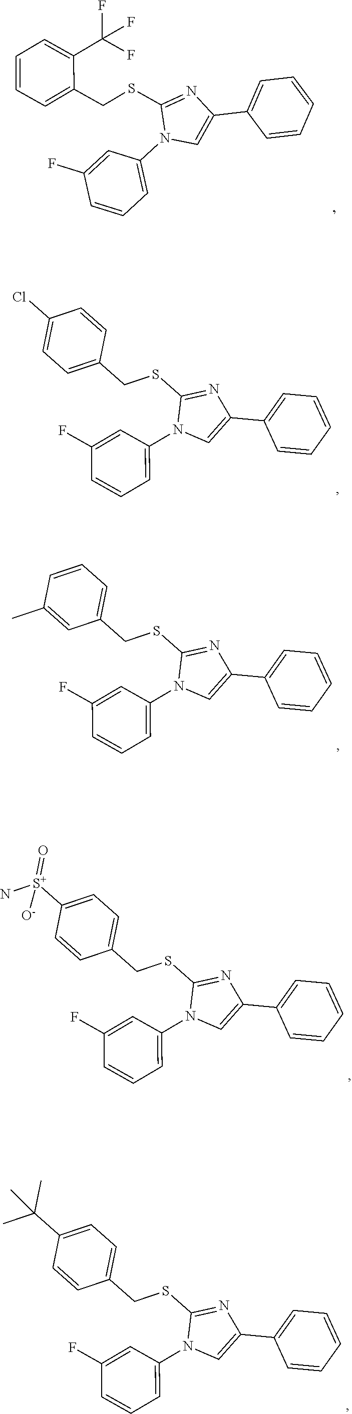

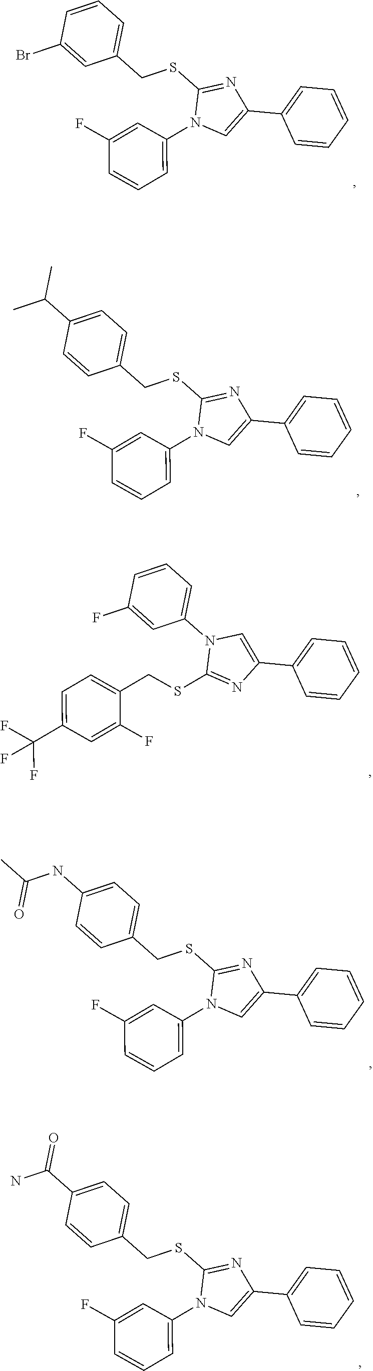

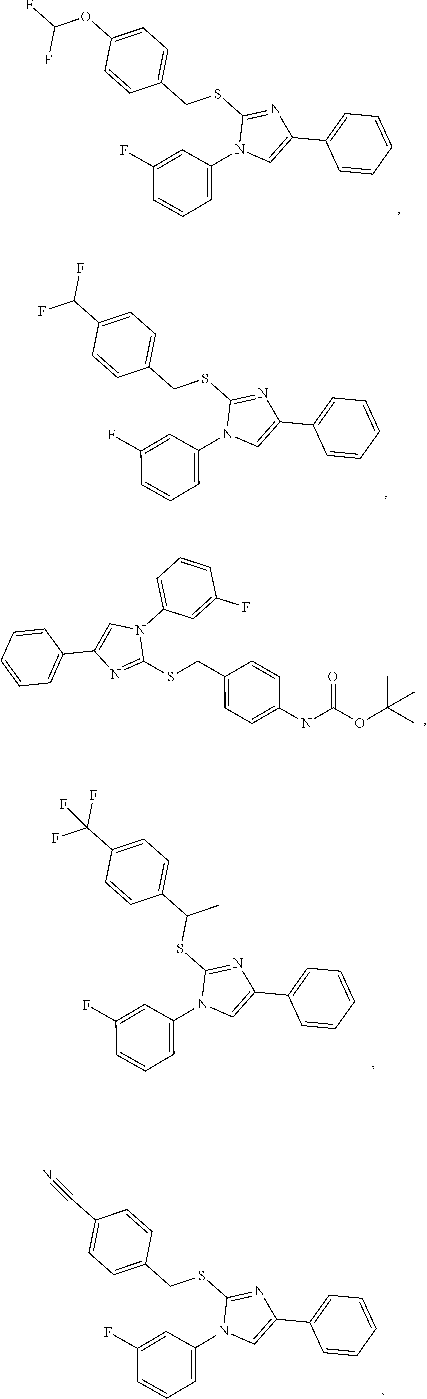

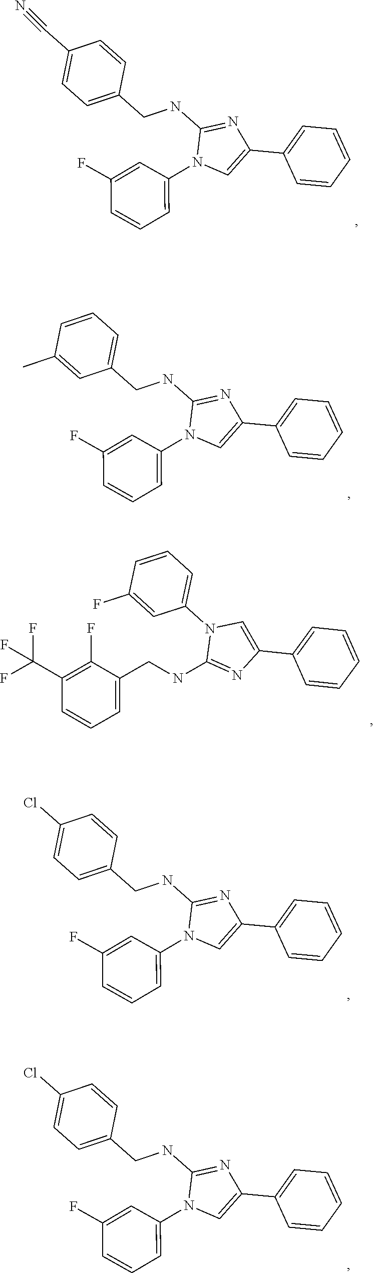

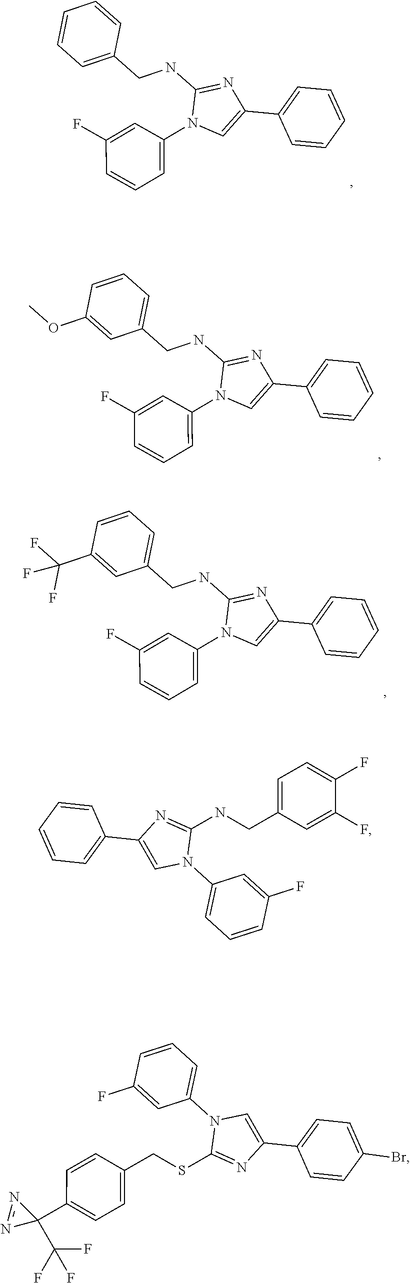

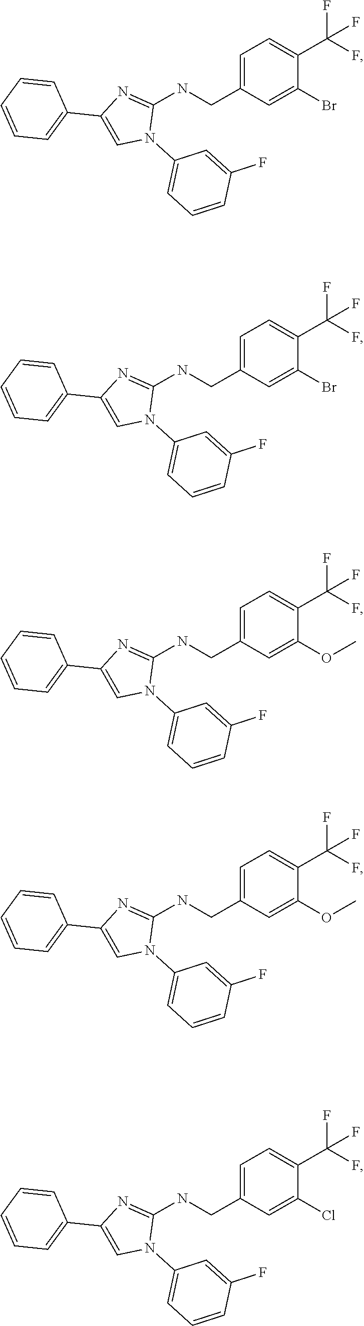

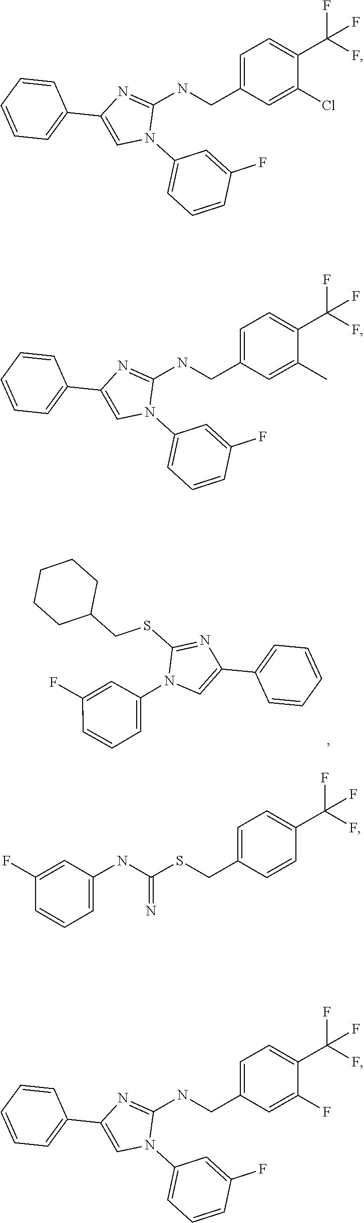

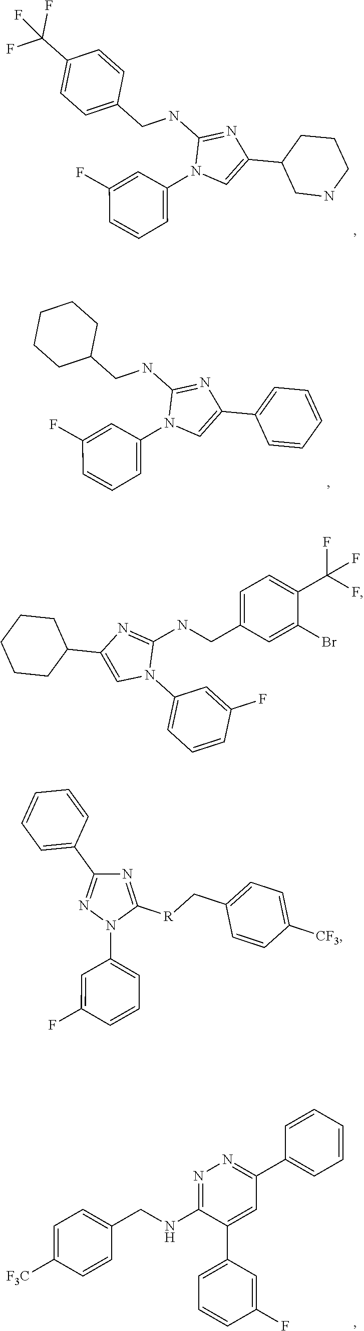

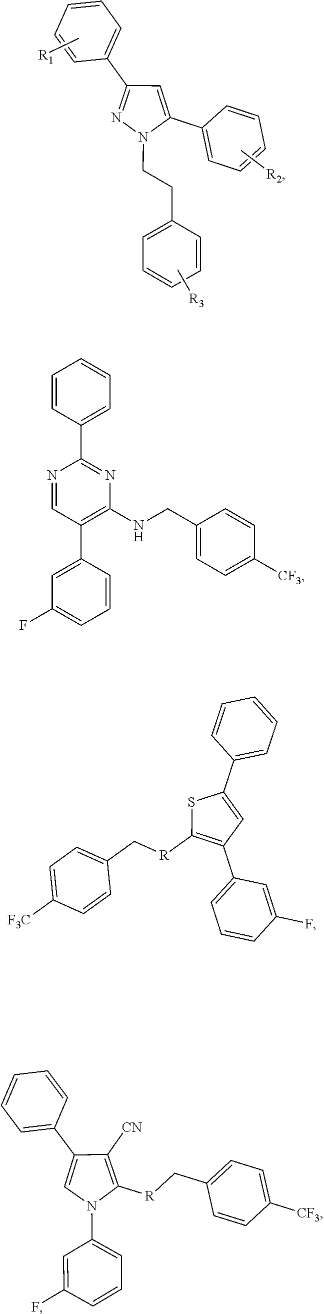

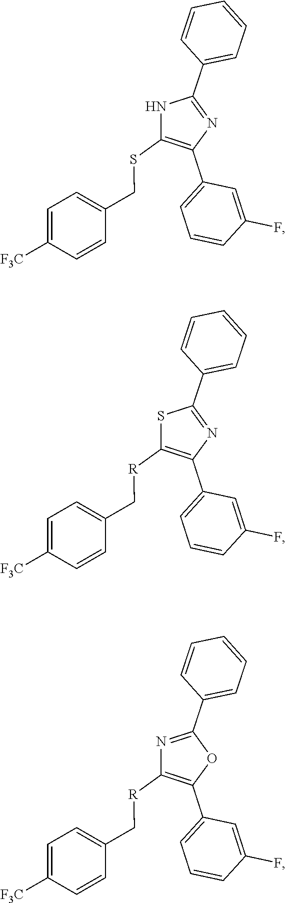

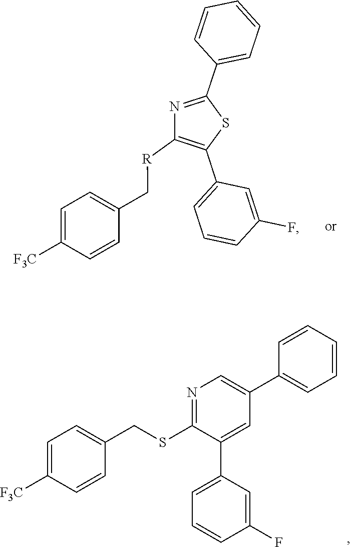

wherein R is S or NH.

The compounds disclosed herein can have a structure below:

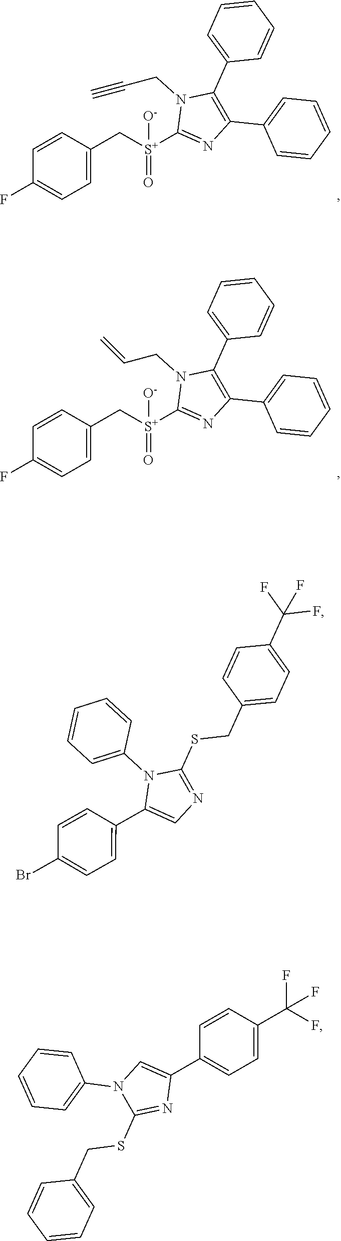

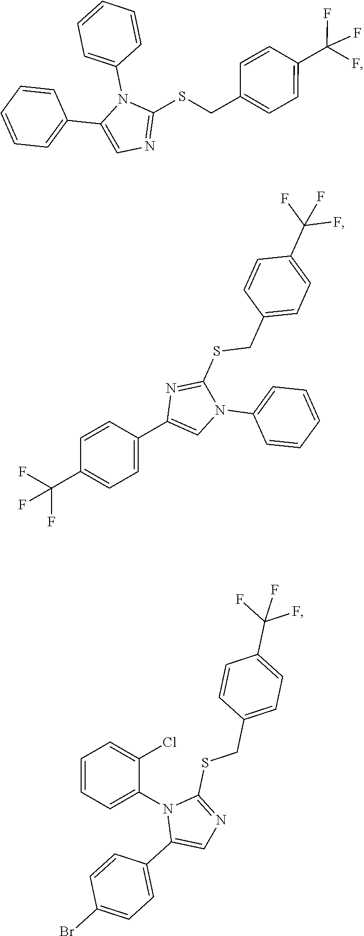

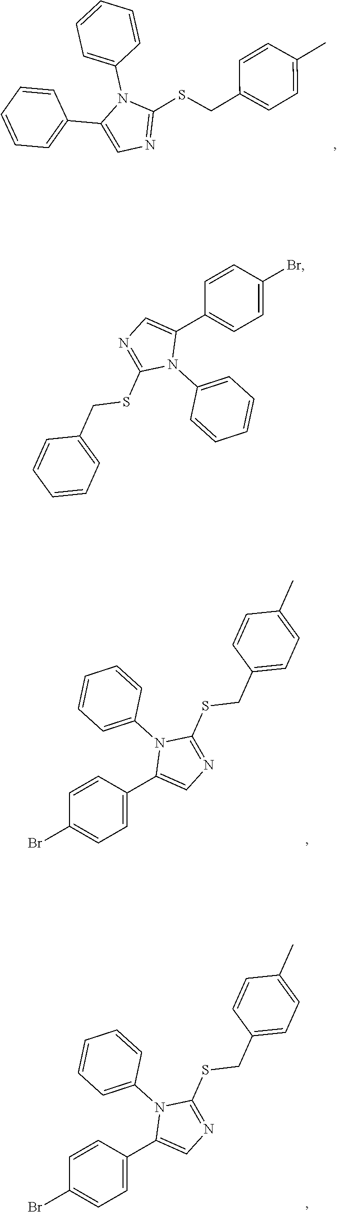

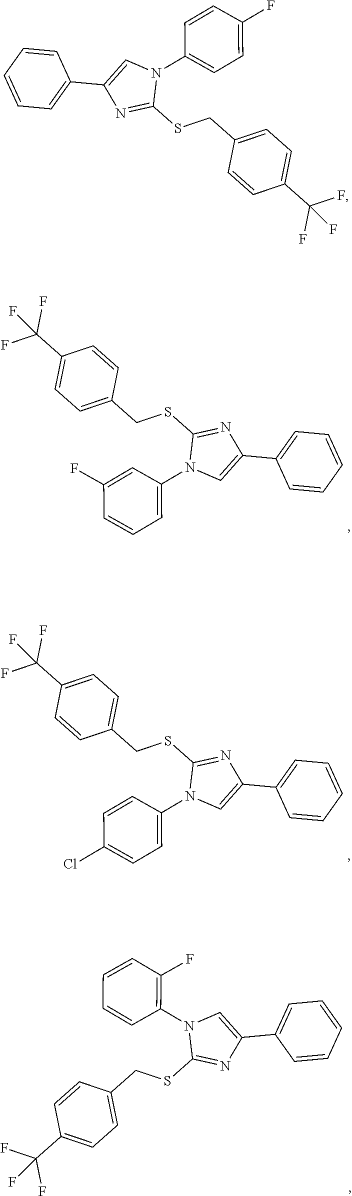

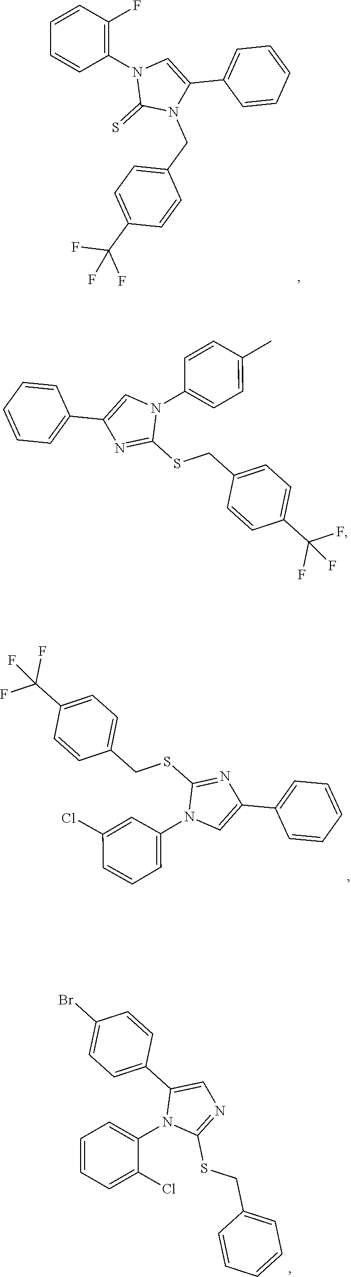

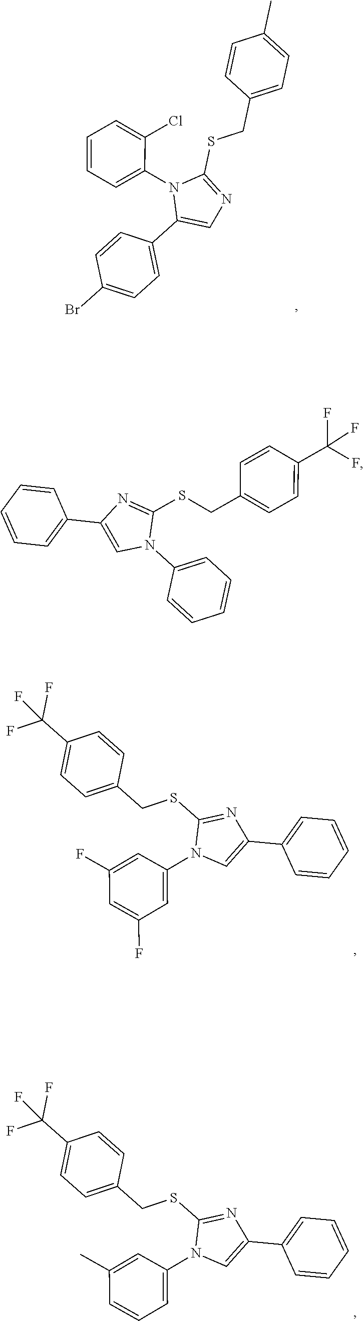

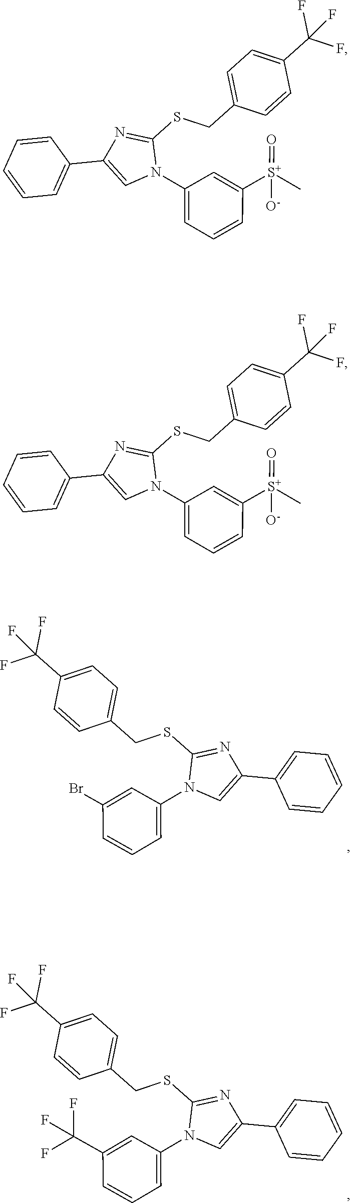

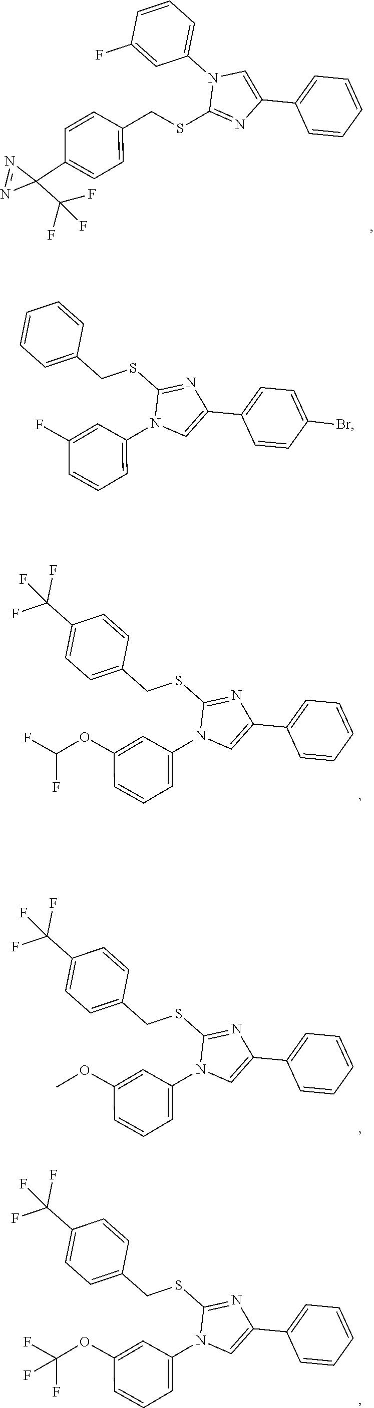

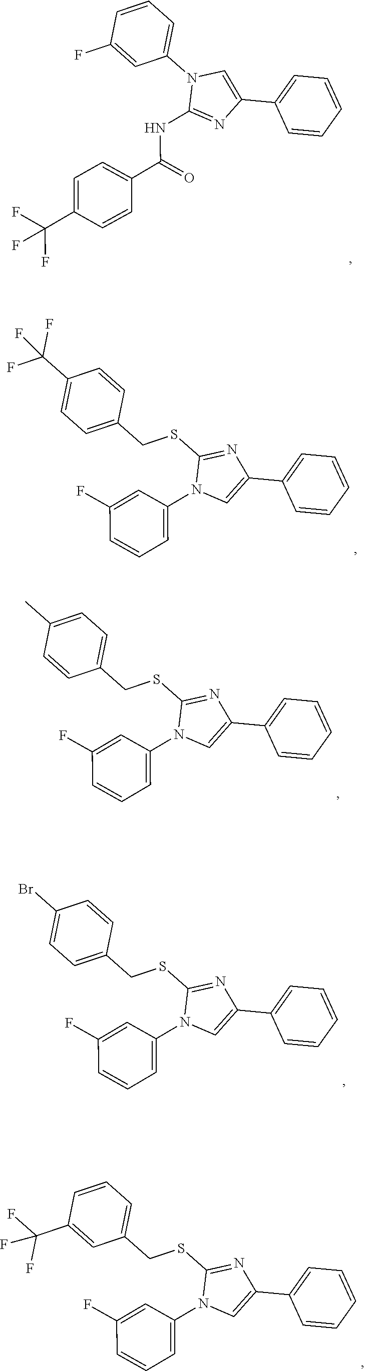

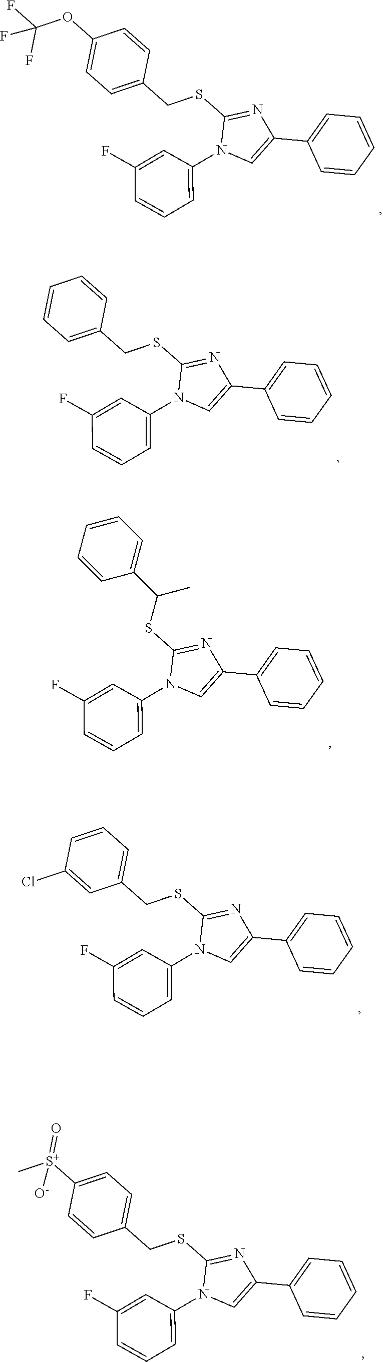

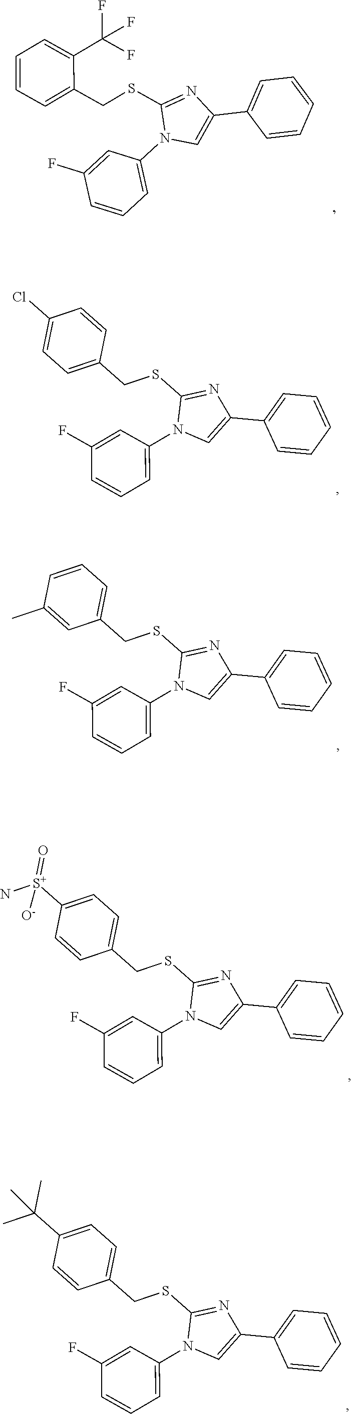

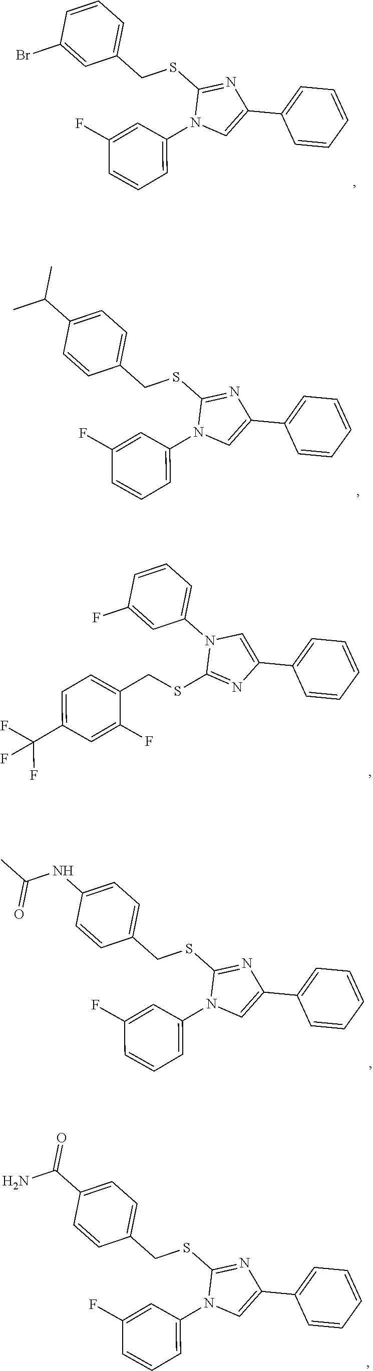

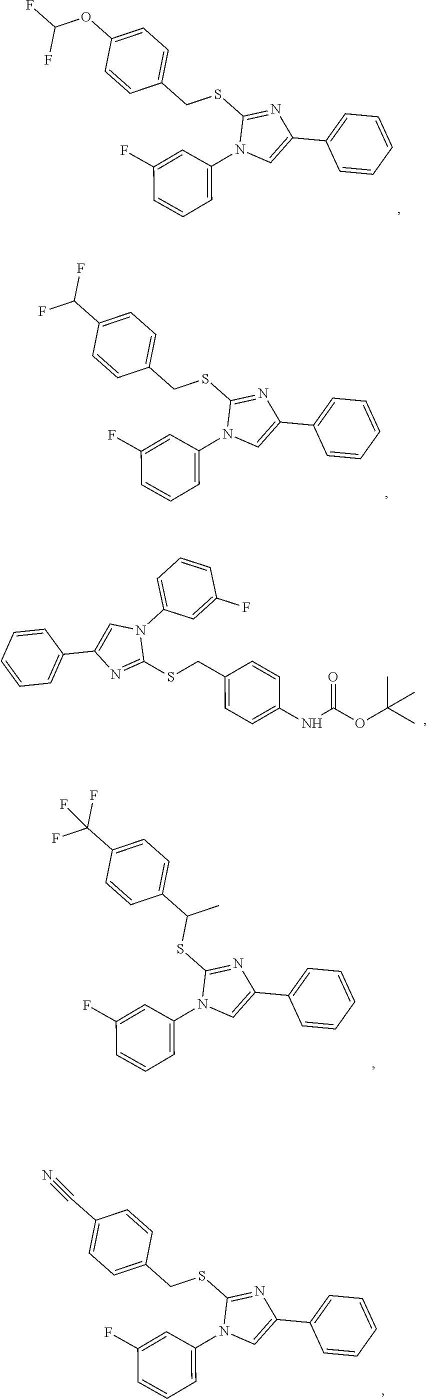

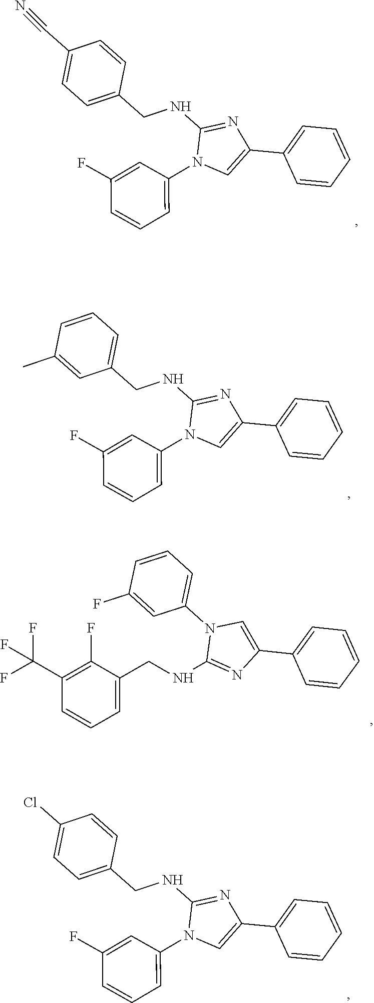

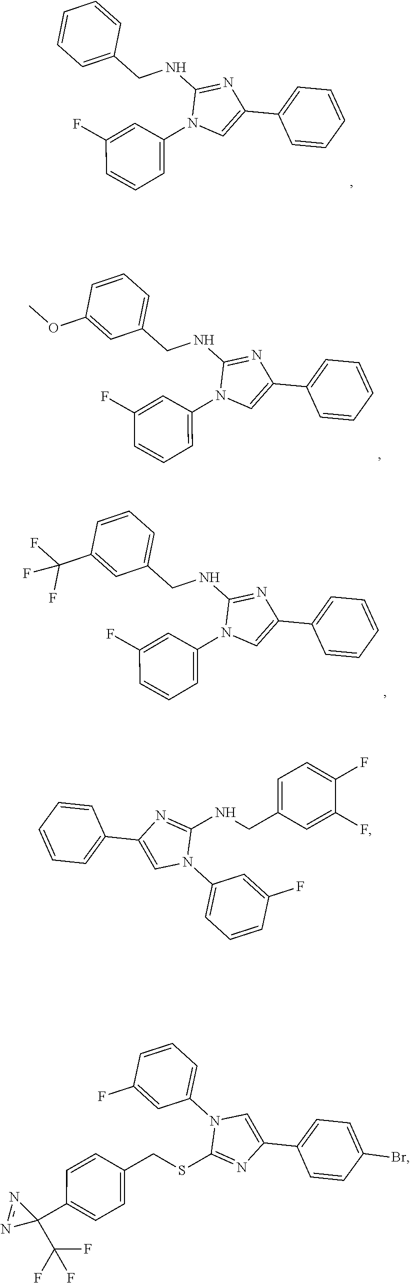

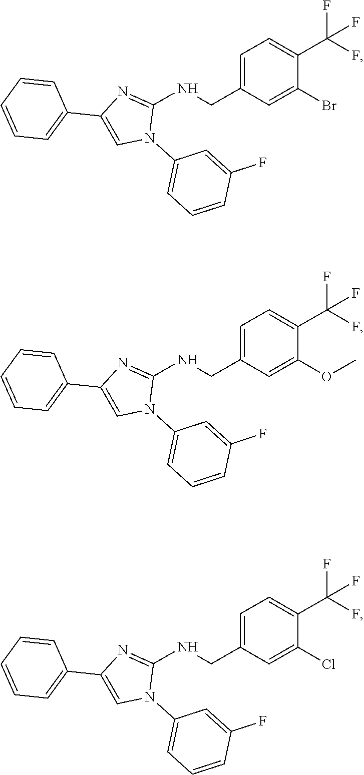

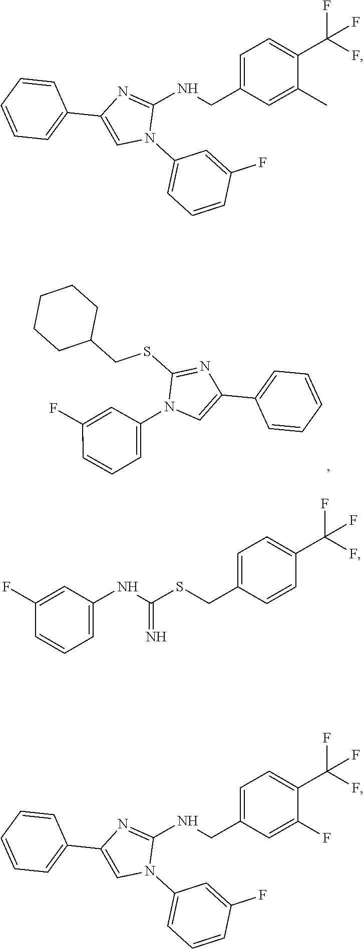

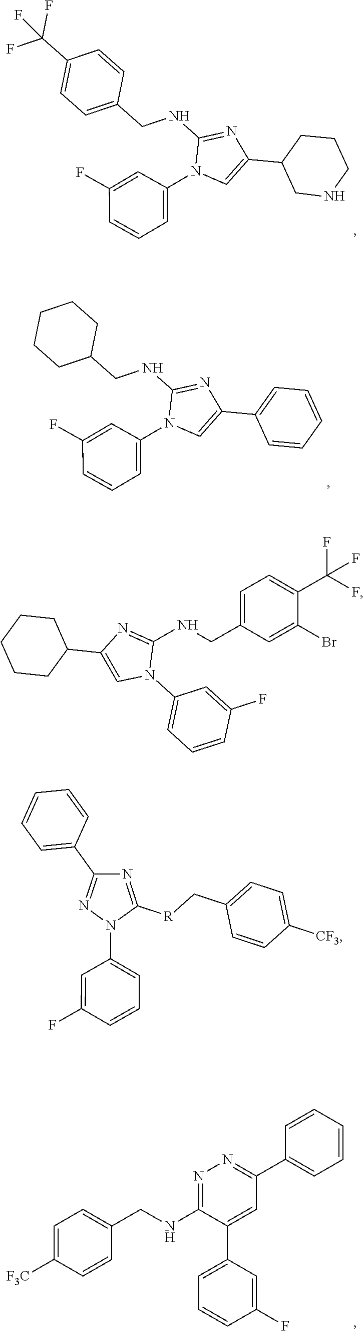

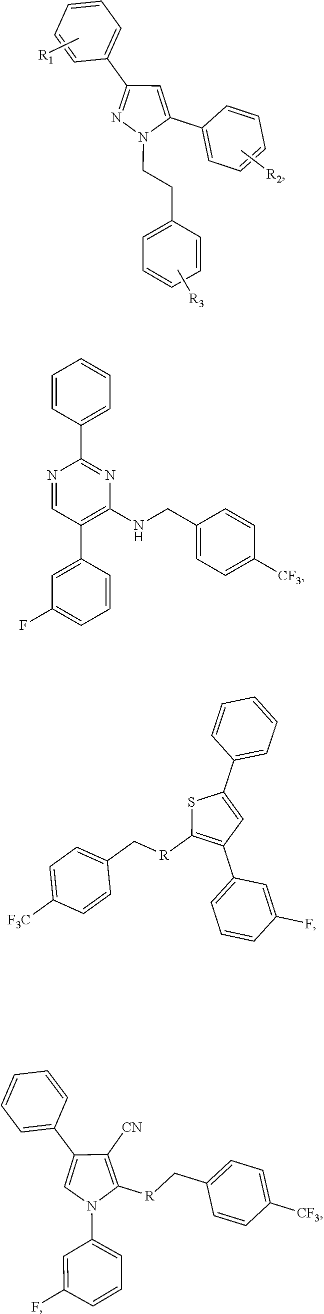

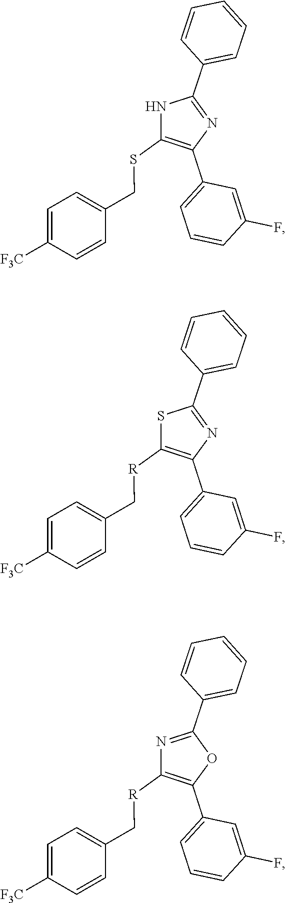

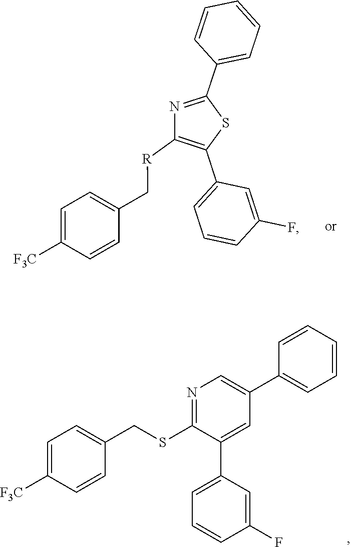

wherein R is S or NH.

In some examples, the compounds disclosed herein can be selected from the group consisting of: 2-((4-fluorobenzyl)sulfonyl)-4,5-diphenyl-1-(prop-2-yn-1-yl)-1H-imidazole; 1-allyl-2-((4-fluorobenzyl)sulfonyl)-4,5-diphenyl-1H-imidazole; 5-(4-bromophenyl)-1-phenyl-2-((4-(trifluoromethyl)benzyl)thio)-1H-imidazole; 2-(benzylthio)-1-phenyl-4-(4-(trifluoromethyl)phenyl)-1H-imidazole; 1,5-diphenyl-2-((4-(trifluoromethyl)benzyl)thio)-1H-imidazole; 1-phenyl-2-((4-(trifluoromethyl)benzyl)thio)-4-(4-(trifluoromethyl)phenyl)-1H-imidazole; 5-(4-bromophenyl)-1-(2-chlorophenyl)-2-((4-(trifluoromethyl)benzyl)thio)-1H-imidazole; 2-((4-methylbenzyl)thio)-1,5-diphenyl-1H-imidazole; 2-(benzylthio)-5-(4-bromophenyl)-1-phenyl-1H-imidazole; 5-(4-bromophenyl)-2-((4-methylbenzyl)thio)-1-phenyl-1H-imidazole; 1-(4-fluorophenyl)-4-phenyl-2-((4-(trifluoromethyl)benzyl)thio)-1H-imidazole; 1-(3-fluorophenyl)-4-phenyl-2-((4-(trifluoromethyl)benzyl)thio)-1H-imidazole; 1-(4-chlorophenyl)-4-phenyl-2-((4-(trifluoromethyl)benzyl)thio)-1H-imidazole; 1-(2-fluorophenyl)-4-phenyl-2-((4-(trifluoromethyl)benzyl)thio)-1H-imidazole; 1-(2-fluorophenyl)-4-phenyl-3-(4-(trifluoromethyl)benzyl)-1,3-dihydro-2H-imidazole-2-thione; 4-phenyl-1-(p-tolyl)-2-((4-(trifluoromethyl)benzyl)thio)-1H-imidazole; 1-(3-chlorophenyl)-4-phenyl-2-((4-(trifluoromethyl)benzyl)thio)-1H-imidazole; 2-(benzylthio)-5-(4-bromophenyl)-1-(2-chlorophenyl)-1H-imidazole; 5-(4-bromophenyl)-1-(2-chlorophenyl)-2-((4-methylbenzyl)thio)-1H-imidazole; 1,4-diphenyl-2-((4-(trifluoromethyl)benzyl)thio)-1H-imidazole; 1-(3,5-difluorophenyl)-4-phenyl-2-((4-(trifluoromethyl)benzyl)thio)-1H-imidazole; 4-phenyl-1-(m-tolyl)-2-((4-(trifluoromethyl)benzyl)thio)-1H-imidazole; 1-(3-(methylsulfonyl)phenyl)-4-phenyl-2-((4-(trifluoromethyl)benzyl)thio)-1H-imidazole; 1-(3-bromophenyl)-4-phenyl-2-((4-(trifluoromethyl)benzyl)thio)-1H-imidazole; 4-phenyl-2-((4-(trifluoromethyl)benzyl)thio)-1-(3-(trifluoromethyl)phenyl)-1H-imidazole; 1-(3-fluorophenyl)-4-phenyl-2-((4-(3-(trifluoromethyl)-3H-diazirin-3-yl)benzyl)thio)-1H-imidazole; 2-(benzylthio)-4-(4-bromophenyl)-1-(3-fluorophenyl)-1H-imidazole; 1-(3-(difluoromethoxy)phenyl)-4-phenyl-2-((4-(trifluoromethyl)benzyl)thio)-1H-imidazole; 1-(3-methoxyphenyl)-4-phenyl-2-((4-(trifluoromethyl)benzyl)thio)-1H-imidazole; 4-phenyl-1-(3-(trifluoromethoxy)phenyl)-2-((4-(trifluoromethyl)benzyl)thio)-1H-imidazole; N-(1-(3-fluorophenyl)-4-phenyl-1H-imidazol-2-yl)-4-(trifluoromethyl)benzamide; 1-(3-fluorophenyl)-4-phenyl-N-(4-(trifluoromethyl)benzyl)-1H-imidazol-2-amine; 1-(3-fluorophenyl)-2-((4-methylbenzyl)thio)-4-phenyl-1H-imidazole; 2-((4-bromobenzyl)thio)-1-(3-fluorophenyl)-4-phenyl-1H-imidazole; 1-(3-fluorophenyl)-4-phenyl-2-((3-(trifluoromethyl)benzyl)thio)-1H-imidazole; 1-(3-fluorophenyl)-4-phenyl-2-((4-(trifluoromethoxy)benzyl)thio)-1H-imidazole; 2-(benzylthio)-1-(3-fluorophenyl)-4-phenyl-1H-imidazole; 1-(3-fluorophenyl)-4-phenyl-2-((1-phenylethyl)thio)-1H-imidazole; 2-((3-chlorobenzyl)thio)-1-(3-fluorophenyl)-4-phenyl-1H-imidazole; 1-(3-fluorophenyl)-2-((4-(methylsulfonyl)benzyl)thio)-4-phenyl-1H-imidazole; 1-(3-fluorophenyl)-4-phenyl-2-((2-(trifluoromethyl)benzyl)thio)-1H-imidazole; 2-((4-chlorobenzyl)thio)-1-(3-fluorophenyl)-4-phenyl-1H-imidazole; 1-(3-fluorophenyl)-2-((3-methylbenzyl)thio)-4-phenyl-1H-imidazole; 4-(((1-(3-fluorophenyl)-4-phenyl-1H-imidazol-2-yl)thio)methyl)benzenesulfonamide; 2-((4-(tert-butyl)benzyl)thio)-1-(3-fluorophenyl)-4-phenyl-1H-imidazole; 2-((3-bromobenzyl)thio)-1-(3-fluorophenyl)-4-phenyl-1H-imidazole; 1-(3-fluorophenyl)-2-((4-isopropylbenzyl)thio)-4-phenyl-1H-imidazole; 2-((2-fluoro-4-(trifluoromethyl)benzyl)thio)-1-(3-fluorophenyl)-4-phenyl-1H-imidazole; N-(4-(((1-(3-fluorophenyl)-4-phenyl-1H-imidazol-2-yl)thio)methyl)phenyl)acetamide; 4-(((1-(3-fluorophenyl)-4-phenyl-1H-imidazol-2-yl)thio)methyl)benzamide; 2-((4-(difluoromethoxy)benzyl)thio)-1-(3-fluorophenyl)-4-phenyl-1H-imidazole; 2-((4-(difluoromethyl)benzyl)thio)-1-(3-fluorophenyl)-4-phenyl-1H-imidazole; tert-butyl (4-(((1-(3-fluorophenyl)-4-phenyl-1H-imidazol-2-yl)thio)methyl)phenyl)carbamate; 1-(3-fluorophenyl)-4-phenyl-2-((1-(4-(trifluoromethyl)phenyl)ethyl)thio)-1H-imidazole; 4-(((1-(3-fluorophenyl)-4-phenyl-1H-imidazol-2-yl)thio)methyl)benzonitrile; 4-(((1-(3-fluorophenyl)-4-phenyl-1H-imidazol-2-yl)amino)methyl)benzonitrile; 1-(3-fluorophenyl)-N-(3-methylbenzyl)-4-phenyl-1H-imidazol-2-amine; N-(2-fluoro-3-(trifluoromethyl)benzyl)-1-(3-fluorophenyl)-4-phenyl-1H-imidazol-2-amine; N-(4-chlorobenzyl)-1-(3-fluorophenyl)-4-phenyl-1H-imidazol-2-amine; N-(4-chlorobenzyl)-1-(3-fluorophenyl)-4-phenyl-1H-imidazol-2-amine; N-benzyl-1-(3-fluorophenyl)-4-phenyl-1H-imidazol-2-amine; 1-(3-fluorophenyl)-N-(3-methoxybenzyl)-4-phenyl-1H-imidazol-2-amine; 1-(3-fluorophenyl)-4-phenyl-N-(3-(trifluoromethyl)benzyl)-1H-imidazol-2-amine; N-(3,4-difluorobenzyl)-1-(3-fluorophenyl)-4-phenyl-1H-imidazol-2-amine; 4-(4-bromophenyl)-1-(3-fluorophenyl)-2-((4-(3-(trifluoromethyl)-3H-diazirin-3-yl)benzyl)thio)-1H-imidazole; N-(3-bromo-4-(trifluoromethyl)benzyl)-1-(3-fluorophenyl)-4-phenyl-1H-imidazol-2-amine; 1-(3-fluorophenyl)-N-(3-methoxy-4-(trifluoromethyl)benzyl)-4-phenyl-1H-imidazol-2-amine; N-(3-chloro-4-(trifluoromethyl)benzyl)-1-(3-fluorophenyl)-4-phenyl-1H-imidazol-2-amine; 1-(3-fluorophenyl)-N-(3-methyl-4-(trifluoromethyl)benzyl)-4-phenyl-1H-imidazol-2-amine; 2-((cyclohexylmethyl)thio)-1-(3-fluorophenyl)-4-phenyl-1H-imidazole; 4-(trifluoromethyl)benzyl (3-fluorophenyl)carbamimidothioate; N-(3-fluoro-4-(trifluoromethyl)benzyl)-1-(3-fluorophenyl)-4-phenyl-1H-imidazol-2-amine; 1-(3-fluorophenyl)-4-(piperidin-3-yl)-N-(4-(trifluoromethyl)benzyl)-1H-imidazol-2-amine; N-(cyclohexylmethyl)-1-(3-fluorophenyl)-4-phenyl-1H-imidazol-2-amine; N-(3-bromo-4-(trifluoromethyl)benzyl)-4-cyclohexyl-1-(3-fluorophenyl)-1H-imidazol-2-amine; 4-(1-(3-fluorophenyl)-2-((4-(trifluoromethyl)benzyl)amino)-1H-imidazol-4-yl)benzonitrile; N-(3-bromo-4-(trifluoromethyl)benzyl)-4-(4-ethynylphenyl)-1-(3-fluorophenyl)-1H-imidazol-2-amine; 4-(4-ethynylphenyl)-1-(3-fluorophenyl)-N-(4-(trifluoromethyl)benzyl)-1H-imidazol-2-amine; 4-(4-ethoxyphenyl)-1-(3-fluorophenyl)-N-(4-(trifluoromethyl)benzyl)-1H-imidazol-2-amine; 1-(3-fluorophenyl)-N-(4-(trifluoromethyl)benzyl)-4-(3-(trifluoromethyl)phenyl)-1H-imidazol-2-amine; N-(3-bromo-4-(trifluoromethyl)benzyl)-4-(4-ethoxyphenyl)-1-(3-fluorophenyl)-1H-imidazol-2-amine; 4-(4-ethoxyphenyl)-1-(3-fluorophenyl)-N-(3-methoxy-4-(trifluoromethyl)benzyl)-1H-imidazol-2-amine; 3,5-diphenyl-1-(4-(trifluoromethyl)benzyl)-1H-pyrazole; 1-(3-fluorophenyl)-4-(tetrahydro-2H-pyran-4-yl)-N-(4-(trifluoromethyl)benzyl)-1H-imidazol-2-amine; 4-(4-bromophenyl)-N-(3-fluoro-4-(trifluoromethyl)benzyl)-1-(3-fluorophenyl)-1H-imidazol-2-amine; 1-phenethyl-3,5-diphenyl-1H-pyrazole; 4-(4-bromophenyl)-1-(3-fluorophenyl)-N-(4-(trifluoromethyl)benzyl)-1H-imidazol-2-amine; 1-(3-fluorophenyl)-4-phenyl-N-((4-(trifluoromethyl)phenyl)methyl-d2)-1H-imidazol-2-amine; 4-(4-bromophenyl)-1-(3-fluorophenyl)-N-(3-methoxy-4-(trifluoromethyl)benzyl)-1H-imidazol-2-amine; 1-(3-fluorophenyl)-N-(3-methoxy-4-(trifluoromethyl)benzyl)-4-(tetrahydro-2H-pyran-4-yl)-1H-imidazol-2-amine; N-(3-fluoro-4-(trifluoromethyl)benzyl)-1-(3-fluorophenyl)-4-(tetrahydro-2H-pyran-4-yl)-1H-imidazol-2-amine; N-(3-bromo-4-(trifluoromethyl)benzyl)-1-(3-fluorophenyl)-4-(tetrahydro-2H-pyran-4-yl)-1H-imidazol-2-amine; 4-(4-chlorophenyl)-1-(3-fluorophenyl)-N-(4-(trifluoromethyl)benzyl)-1H-imidazol-2-amine; 4-(4-ethynylphenyl)-1-(3-fluorophenyl)-N-(3-methoxy-4-(trifluoromethyl)benzyl)-1H-imidazol-2-amine; 1-phenyl-N-(4-(trifluoromethyl)phenethyl)-1H-pyrazol-4-amine; 1-phenyl-N-(4-(trifluoromethyl)phenethyl)-1H-pyrazol-3-amine; 1-(3-bromophenyl)-N-(3-methoxy-4-(trifluoromethyl)benzyl)-4-phenyl-1H-imidazol-2-amine; N-(3-methoxy-4-(trifluoromethyl)benzyl)-4-phenyl-1-(m-tolyl)-1H-imidazol-2-amine; 1,4-diphenyl-N-(4-(trifluoromethyl)benzyl)-1H-pyrazol-3-amine; 4-(3-fluorophenyl)-1-phenyl-N-(4-(trifluoromethyl)benzyl)-1H-pyrazol-3-amine; 3,5-diphenyl-1-(4-(trifluoromethyl)phenethyl)-1H-pyrazole; 4-(3,5-difluorophenyl)-1-phenyl-N-(4-(trifluoromethyl)benzyl)-1H-pyrazol-3-amine; 5-(3-fluorophenyl)-1-phenyl-N-(4-(trifluoromethyl)phenethyl)-1H-pyrazol-4-amine; 1,5-diphenyl-3-((4-(trifluoromethyl)benzyl)thio)-1H-1,2,4-triazole; 1,3-diphenyl-5-((4-(trifluoromethyl)benzyl)thio)-1H-1,2,4-triazole; a pharmaceutically acceptable salt, ester, or prodrug thereof; and combinations thereof.

Methods of Use

Methods for treating or preventing necroinflammation in a subject are disclosed herein. Necroinflammation is the immune response to necrosis in a living organism. Necrosis is executed as a regulated process through defined signaling pathways such as necroptosis, ferroptosis, and pyroptosis or may happen in a nonregulated fashion as traumatic necrosis. Ferroptosis is an iron-dependent form of programmed cell death and is pathogenic to several acute and chronic diseases including asthma, renal failure, neurodegenerative diseases, injury by ionizing radiation, and brain trauma. Ferroptosis is executed via oxygenation of polyunsaturated phosphatidylethanolamines (PE) by 15-lipoxygenases (15-LO) that normally use free polyunsaturated fatty acids as substrates. Specifically, it has been shown that PEBP1, a scaffold protein inhibitor of protein kinase cascades, complexes with 15LO1 and 15LO2, and changes their substrate competence to generate hydroperoxy-PE. Inadequate reduction of hydroperoxy-PE due to insufficiency or dysfunction of a selenoperoxidase, GPX4, leads to ferroptosis. 15LOX/PEBP1-dependent regulatory mechanisms of ferroptotic death in airway epithelial cells in asthma, kidney epithelial cells in renal failure, cortical and hippocampal neurons in brain trauma, intestinal epithelial cells in radiation disease among other relevant conditions, represent important targets for drugs.

In some aspects, the methods and compounds disclosed herein are for preventing or treating necroinflammation associated with ferroptotic processes by inhibiting 15 lipoxygenase/phosphatidylethanolamine binding protein (15LOX/PEBP1) complexes, wherein the inhibitor exhibits a higher binding affinity or binding activity for the 15LOX/PEBP1 complex compared to 15LOX alone. In some other aspects, the methods and compounds disclosed herein are for preventing or treating necroinflammation associated with ferroptotic processes by inhibiting interaction of 15 lipoxygenase (15LOX) with phosphatidylethanolamine binding protein PEBP1. 15 lipoxygenase as described herein includes two 15LO isoforms, namely 15 lipoxygenase 1 (15LO1) and 15 lipoxygenase 2 (15LO2) and both forms are considered in the methods disclosed herein. In further aspects, the methods and compounds disclosed herein are for preventing or treating necroinflammation associated with ferroptotic processes by inhibiting accumulation of 15-hydroperoxy-eicasotetraenoyl-phosphatidylethanolamines (15 HpETE-PE).

In specific aspects, provided herein are methods for preventing or treating necroinflammation associated with ferroptotic processes causing renal injury in a subject in need thereof. The method can comprise inhibiting 15 lipoxygenase/phosphatidylethanolamine binding protein (15LOX/PEBP1) complex, inhibiting interaction of 15 lipoxygenase (15LOX) with phosphatidylethanolamine binding protein PEBP1, or inhibiting accumulation of 15 HpETE-PE in the subject. In particular embodiments, the methods are directed to protecting a kidney from acute renal injury or from chronic renal injury. The renal injury can be associated with the pre-existence of one or more known risk factors for prerenal, intrinsic renal, or postrenal failure in the subject. In some examples, the renal injury can be associated with exertional rhabdomyolysis, dehydration, a crush-injury, blood loss, burn, sepsis, or an existing diagnosis of one or more of congestive heart failure, preeclampsia, eclampsia, diabetes mellitus, hypertension, coronary artery disease, proteinuria, renal insufficiency, glomerular filtration below the normal range, cirrhosis, serum creatinine above the normal range, injury to renal function, reduced renal function, or acute renal failure, or based on undergoing or having undergone major vascular surgery, coronary artery bypass, or other cardiac surgery, or based on exposure to a nephrotoxic agent such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin. The methods of preventing and treating necroinflammation associated with ferroptotic processes causing renal injury can further comprise administering to the subject an additional therapeutically active co-agent used in the treatment of renal injury.

In other specific aspects, provided herein are methods for preventing or treating necroinflammation associated with ferroptotic processes causing an upper or lower respiratory disorders in a subject in need thereof. The method can comprise inhibiting 15 lipoxygenase/phosphatidylethanolamine binding protein (15LOX/PEBP1) complexes, inhibiting interaction of 15 lipoxygenase (15LOX) with phosphatidylethanolamine binding protein PEBP1, or inhibiting accumulation of 15 HpETE-PE in the subject. In particular embodiments, the upper or lower respiratory disorder is asthma, sinusitis, nasal polyps, COPD, bronchitis, bronchiectasis, or cystic fibrosis. In some examples, the methods treats or prevents exacerbation prone asthma. The methods of preventing and treating necroinflammation associated with ferroptotic processes causing upper or lower respiratory disorders can further comprise administering an additional therapeutically active co-agent used in the treatment of the respiratory disorder.

In other specific aspects, provided herein are methods for preventing or treating necroinflammation associated with ferroptotic processes causing an acute or chronic brain injury in a subject in need thereof. The method can comprise inhibiting 15 lipoxygenase/phosphatidylethanolamine binding protein (15LOX/PEBP1) complexes, inhibiting interaction of 15 lipoxygenase (15LOX) with phosphatidylethanolamine binding protein PEBP1, or inhibiting accumulation of 15 HpETE-PE in the subject. In particular embodiments, the methods result in a decrease in tissue damage and/or enhance recovery post-injury. For example, the methods can suppress traumatic brain injury-induced neuronal death. In some embodiments, the methods preserve or restore at least a portion of motor function, sensory function, cognitive function, visual function, auditory function, or a combination thereof in the subject. The methods of preventing and treating necroinflammation associated with ferroptotic processes causing an acute or chronic brain injury can further comprise administering an additional therapeutically active co-agent used in the treatment of the traumatic brain injury.

In other specific aspects, provided herein are methods for preventing or treating necroinflammation associated with ferroptotic processes causing injury by radiation in a subject in need thereof. The method can comprise inhibiting 15 lipoxygenase/phosphatidylethanolamine binding protein (15LOX/PEBP1) complexes, inhibiting interaction of 15 lipoxygenase (15LOX) with phosphatidylethanolamine binding protein PEBP1, or inhibiting accumulation of 15 HpETE-PE in the subject. In particular embodiments, the methods are directed to protecting a subject from injury associated with partial or full body radiation.

In other specific aspects, provided herein are methods for preventing or treating necroinflammation associated with ferroptotic processes causing a neurodegenerative disorder in a subject in need thereof. The method can comprise inhibiting 15 lipoxygenase/phosphatidylethanolamine binding protein (15LOX/PEBP1) complexes, inhibiting interaction of 15 lipoxygenase (15LOX) with phosphatidylethanolamine binding protein PEBP1, or inhibiting accumulation of 15 HpETE-PE in the subject. In particular embodiments, the methods are directed to protect against degradation of neurons (e.g., cerebellum, spinal cord, and surrounding neurons (e.g., neuromuscular synapses), more typically brain and spinal cord neurons, or in a preferred embodiment, the degradation of neurons in the brain). Neurodegenerative diseases may include Alzheimer's disease; Huntington's disease; Parkinson's disease, Kennedy's disease, frontotemporal dementia, ischemic stroke, hemorrhagic stroke, global cerebral ischemia-reperfusion, a prion-related disorder, and/or similar neurodegenerative condition. Neurodegenerative diseases may also include exposure to seizures, heat stress, radiation, toxins, infection, injury, and the degradation of neurons.

In some aspect, disclosed are methods for preventing or treating necroinflammation associated with ferroptotic processes causing an upper or lower respiratory disorder, an acute or chronic brain injury, renal failure, a radiation induced injury, or a combination thereof in a subject by administration of one or more of the compounds disclosed herein and optionally a combination of at least one compound or composition as disclosed herein and at least one active co-agent. Administration may be accomplished via direct immersion; systemic or localized intravenous (i.v.), intraperitoneal (i.p.), subcutaneous (s.c.), intramuscular (i.m.), or direct injection into an organ; and/or by oral or inhaled/nebulized administration of the appropriate formulations.

Administration

The term “administration” and variants thereof (e.g., “administering” a compound) in reference to a compound disclosed herein means introducing the compound or a prodrug of the compound into the system of the animal in need of treatment. When a compound or prodrug thereof is provided in combination with one or more other active agents, “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, inhaled, nasal, rectal, topical, and parenteral routes of administration. As used herein, the term parenteral includes subcutaneous, intradermal, intravenous, intramuscular, intraperitoneal, intraventricular and intrasternal administration, such as by injection. Administration of the disclosed compounds or compositions can be a single administration, or at continuous or distinct intervals as can be readily determined by a person skilled in the art.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer; poly[bis(p-carboxyphenoxy) propane:sebacic acid] in a 20:80 molar ratio (as used in GLIADEL); chondroitin; chitin; and chitosan.

For the treatment of necroinflammation associated with ferroptotic processes causing an upper or lower respiratory disorder, an acute or chronic brain injury, renal failure, a radiation induced injury, neurodegenerative disorder, or a combination thereof, the compounds disclosed herein can be administered to a patient in need of treatment in combination with other active coagents. These other substances or treatments can be given at the same as or at different times from the compounds disclosed herein.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of injury (such as a site of brain injury), optionally in combination with a pharmaceutically acceptable carrier such as an inert diluent. Compounds and compositions disclosed herein can be systemically administered, such as intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally, intraventricularly, by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For administration by inhalation, the composition can be conveniently delivered from an insufflator, nebulizer pressurized packs or other convenient means of delivering an aerosol spray. Pressurized packs can comprise a suitable propellant such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Alternatively, for administration by inhalation or insufflation, the compounds can take the form of a dry powder composition, for example a powder mix of the compound and a suitable powder base such as lactose or starch. The powder composition can be presented in unit dosage form, in for example, capsules, cartridges, gelatin or blister packs from which the powder can be administered with the aid of an inhalator or insufflator.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of traumatic brain injury, respiratory disorder, renal injury, injury by ionizing radiation, or neurodegenerative disorders, compounds and agents and compositions disclosed herein can be administered to a patient in need of treatment prior to, subsequent to, or in combination with other active agents that can treat the condition or disorders.

Kits

Kits for practicing the methods of the invention are further provided. By “kit” is intended any manufacture (e.g., a package or a container) comprising at least one reagent, e.g., anyone of the compounds described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight. The precise amount of composition administered to an individual will be the responsibility of the attendant physician. The specific dose level for any particular individual will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration can vary depending on the condition and its severity. The dosage can be increased or decreased over time, as required by an individual. An individual initially can be given a low dose, which is then increased to an efficacious dosage tolerable to the individual.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

Traumatic brain injury (TBI) is reported to be the leading cause of death and disability in children around the world. Each year in the US, severe TBI in children results in ˜7400 deaths and 60,000 hospitalizations. Fifty percent of surviving children with severe TBI have poor neurological outcome at six months. Severe TBI in children is thus a critical problem in desperate need of impactful therapies. Free radicals and oxidative stress have been uniformly accepted as universal pathogenic mechanisms of TBI prompting therapeutic use of antioxidants. Invariably, clinical trials of non-specific free radical scavengers/antioxidants failed. This suggests that true sources and mechanisms of TBI redox disbalance remain undefined, and represent a potential therapeutic opportunity. It has been shown that lipid peroxidation after TBI in immature brain occurs as a result of controlled enzymatic reactions. Peroxidation of mitochondrial phospholipid cardiolipin (CL) represents a required stage of neuronal apoptosis after TBI in postnatal day (PND) 17 rats. Cytochrome c was identified as a catalyst of CL peroxidation and showed that a mitochondria targeted inhibitor of CL peroxidation suppressed TBI-induced apoptosis and preserved cognitive function in PND17 rats. Highly selective oxidation of arachidonic acid (AA) containing phosphatidylethanolamines (PE) by 15 lipoxygenase (15LOX) was identified to be causative to ferroptosis. It was discovered that PE binding protein 1 (PEBP1) complexes with 15LOX and changes its substrate specificity from free AA to AA esterified into PE to generate hydroperoxy-AA-PE death signals. Normally hydroperoxy-AA-PE are eliminated by combined action of glutathione peroxidase 4 (GPX4)/glutathione (GSH). Immature brain has lower GSH levels and GPX activity vs adult brain thus could be more vulnerable to ferroptosis upon injury. Indeed, data show that TBI leads to marked increase in expression and activity of 15LOX, and accumulation of oxidized AA-PE in PND17 rat brain. Furthermore, the data provided herein indicate that inhibition of AA-PE oxidation suppresses TBI-induced neuronal death and preserves cognitive function. Thus, it can be said that generation of oxidized AA-PE by 15LOX/PEBP1 complex leads to neuronal death and represents a new target for drug discovery. This premise can be analyzed as follows:
a) Determine the degree, spatial and temporal pattern of 15LOX/PEBP1 complex formation and AA-PE oxidation after TBI.
B) Investigate the mechanisms of AA-PE oxidation in TBI-induced neuronal death.
c) Create and investigate the mechanism of action and neuroprotective potential of small-molecule regulators of 15LOX and 15LOX/PEBP1 activity in TBI.

Lipidomics and oxidative lipidomics technology can be utilized to provide important mechanistic information on the role of PE oxidation in neuronal ferroptosis after pediatric TBI. The ability to selectively modulate PE oxidation, a significant early event in the mechanism of ferroptosis, could lead to targeted therapies for TBI and ultimately improve outcome for children after brain injury.

Methodology

a) 15LOX/PEBP1 Complex Formation and AA-PE Oxidation after TBI.

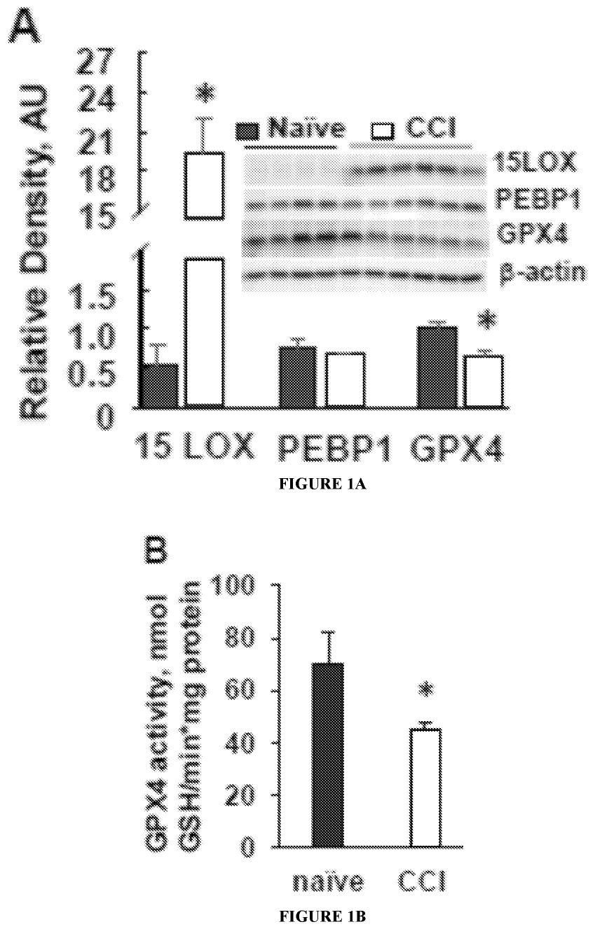

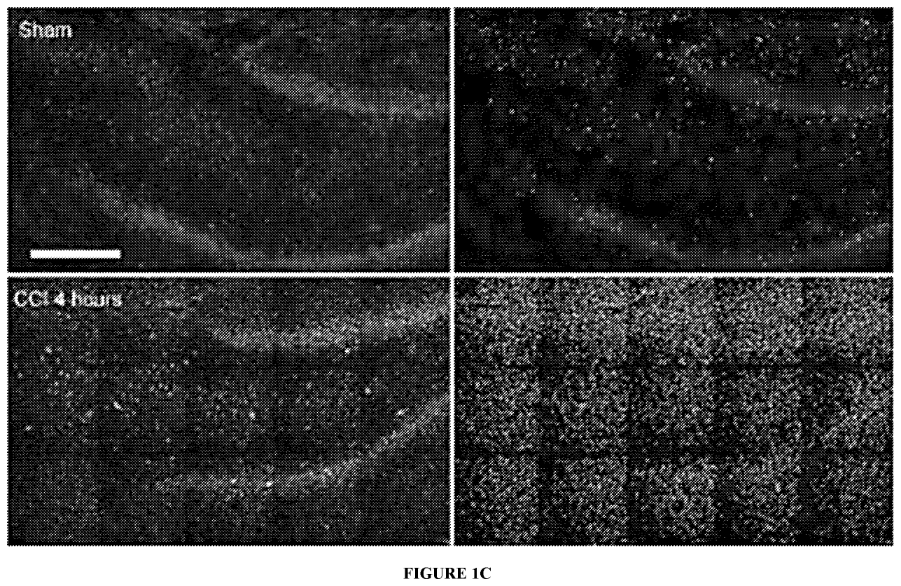

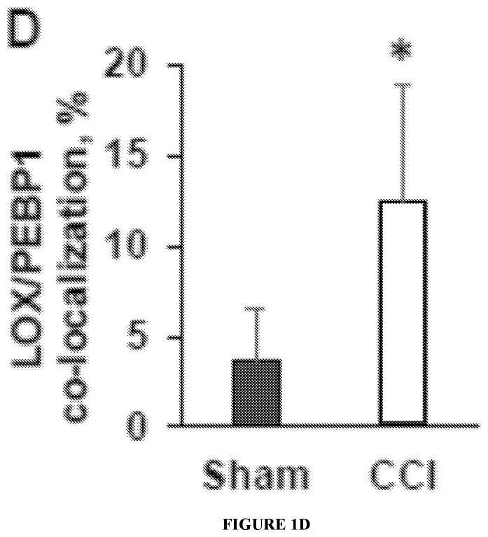

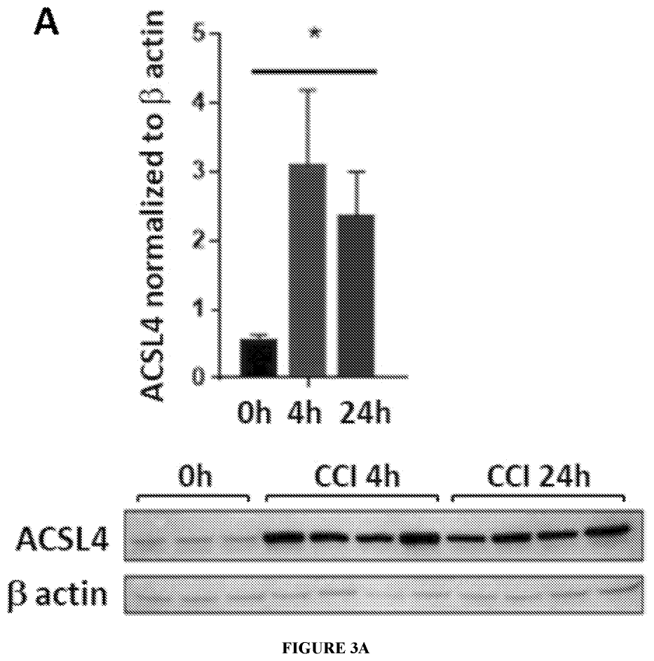

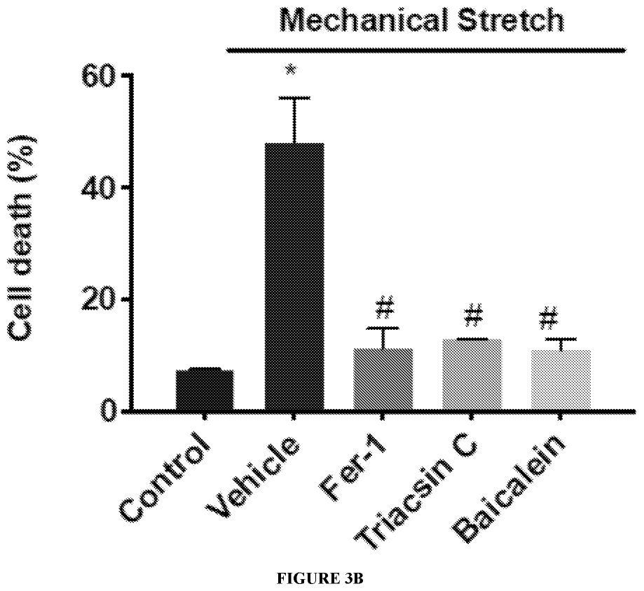

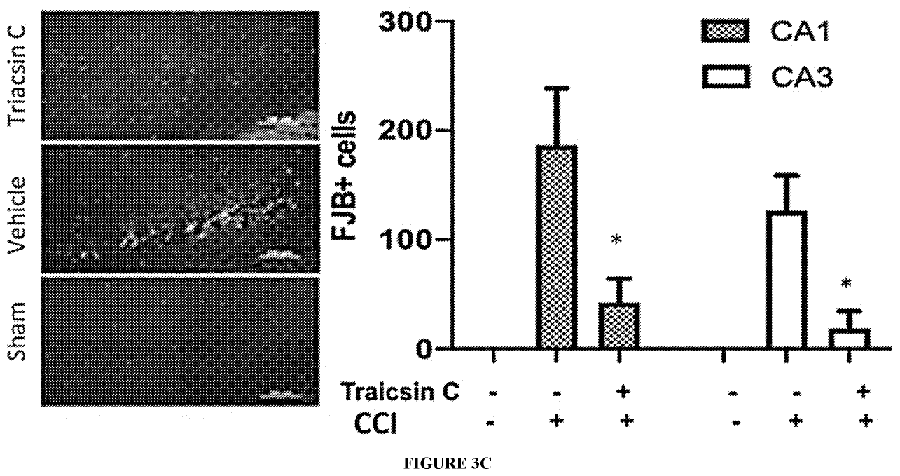

PND17 rat CCI model was utilized to detect changes in pro-ferroptotic proteins and oxygenated AA-PE after injury. At 4 h after CCI, ipsilateral cortical expression of 15LOX was markedly elevated vs control rats (FIG. 1A) whereas GPX4 levels and its enzymatic activity (FIG. 1A, FIG. 1B) were decreased. Similar protein profiles were observed in ipsilateral hippocampus after CCI. High-resolution large area confocal immuno-fluorescence microscopy assessments of PEBP1/15LOX co-localizations (FIG. 1C) showed a remarkably higher abundance of co-localized puncta in the brain of injured vs. sham rats. Overall, the protein profile in injured brain reflected a shift toward a pro-oxidative lipid environment (15-LOX and ACSL4 (FIG. 3A)) without a compensatory increase in lipid-hydroperoxide reducing capacity (GPX4 and GSH77).

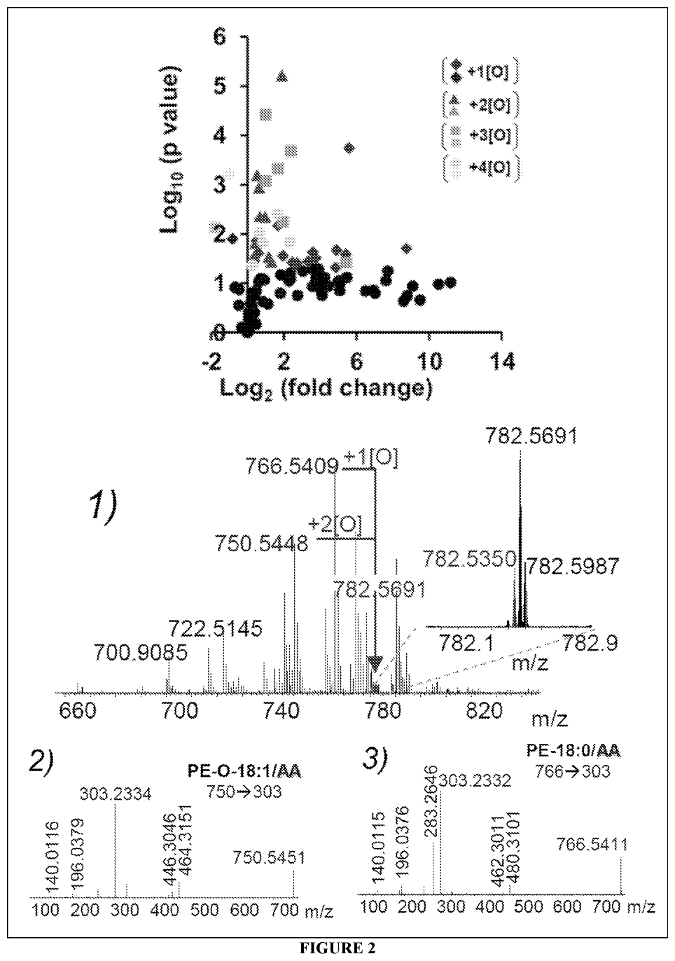

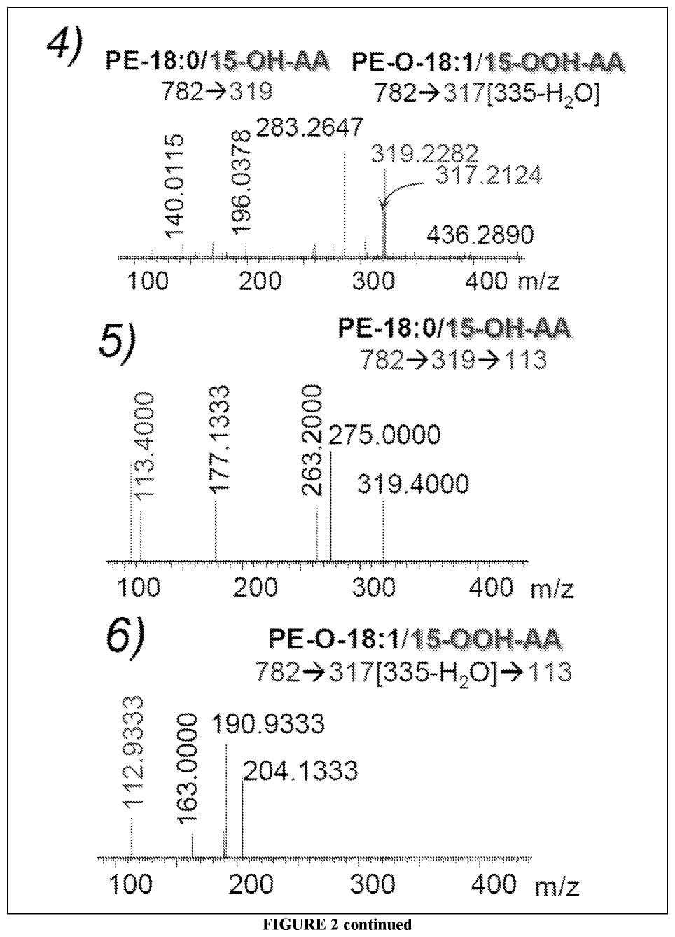

FIG. 2 shows a significant increase in PEox in contusional cortex after CCI vs. sham controls. MS/MS fragmentation analyses of PEox demonstrated a predominance of 15-LOX product 15-HOO-AA-PE (FIG. 2).

Time course studies (0, 4, 8, 24 and 72 h) of TBI-induced changes in i) PE oxidation (oxidative lipidomics); ii) expression and activity of 15LOX, PEBP1, ACSL4, LPCAT3, iPLA2β and GPX4 (western blot); iii) formation of 15LOX/PEBP1 complexes (dual object recognition); and iv) GSH levels (fluorescent assay) will be performed as follows. An established model of in vitro TBI, severe mechanical stretch injury in primary cortical neurons, can be used. In brief, cortical neurons are cultured on 0.05% poly-D-lysine hydrobromide coated silicone substrate wells, and subjected to a computer controlled quantifiable mechanical insult by displacing the silicone substrate over a hollowed platform. In vivo TBI is induced by severe CCI (6-mm impactor tip, velocity of ±0.2 m/secs, deformation depth of 2.5 mm, impact duration of 50 msecs) to left parietal cortex in male and female PND 17 rats. To maximize rigor and reproducibility, animals are assigned to either sham (anesthesia and craniotomy) or CCI groups by block randomization. To ensure appropriate sample size for robustness, power analyses are performed in each experimental context. A sample size of 5 is preferred to detect a 25% difference in PE-OOH between CCI and control based on standard deviation observed in the experiments with an alpha of 0.05 and power of 0.80. Group assignments are blinded to the investigators who perform biochemical and histological endpoints. Statistical differences are analyzed using one-way ANOVA with post-hoc comparisons between groups if overall p<0.05 as appropriate. To address biological variability, both sexes are included.

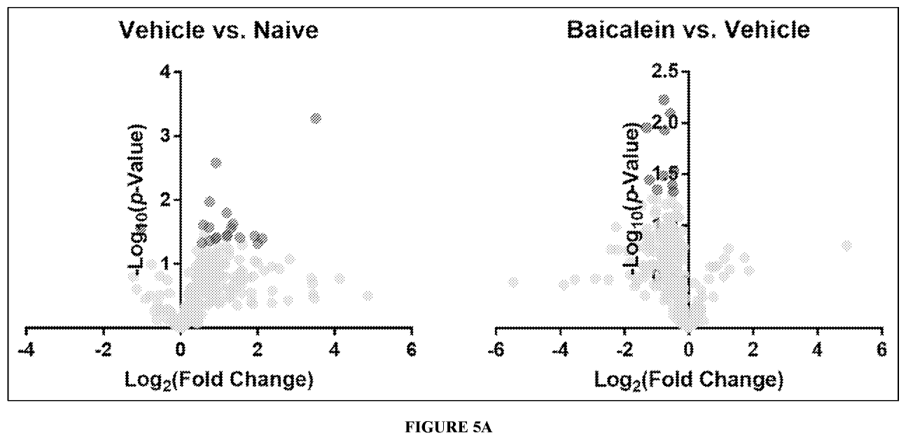

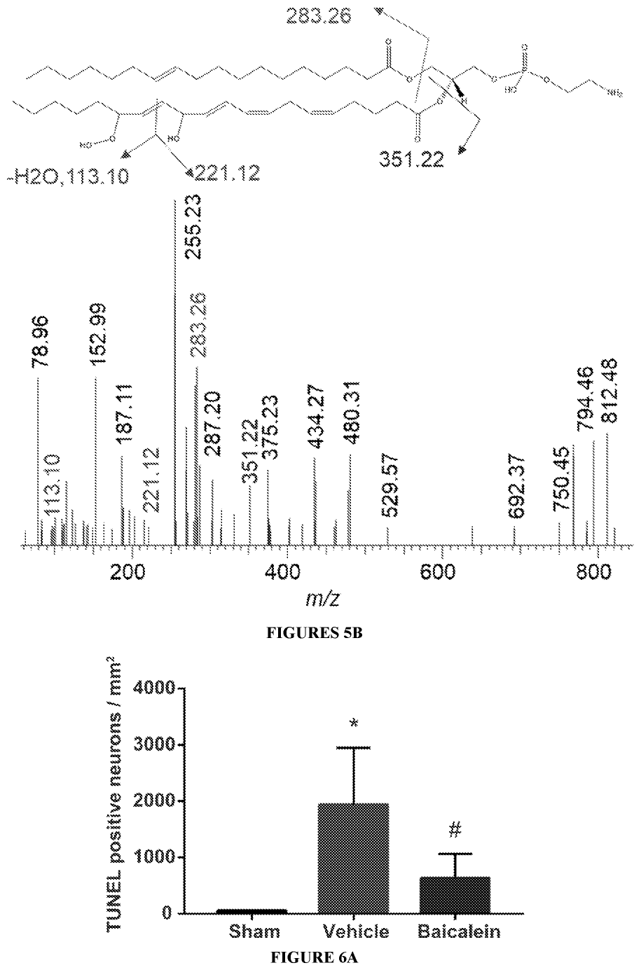

PE oxidation is quantified using oxidative phospholipidomics that uses combination of various LC and MS methodologies. Substrate specificity is determined for fatty acid-speciation of all PLs as well as towards different major classes: phosphatidylcholine (PC), PE, phosphatidylserine (PS), phosphatidic acid (PA) phosphatidylglycerol (PG), phosphatidylinositol (PI), CL. Differences in chemical nature of PL oxidation products (with varying number and positions of oxygen atoms in the fatty acid chains) are characterized. Tandem MS/MS analysis is performed using the most advanced Fusion Lumos Orbitrap LC-MS (Thermo Fisher Scientific) that permits unlimited fragmentation in its ion-trap and establish the structure of oxidation products in spite of their low abundance (FIG. 2, FIG. 5B).

Object based co-localization analyses of 15LOX/PEBP1 complex is then performed. Briefly, 3D confocal (Nikon A1, 60×, 1.4NA) stacks (200 nm optical sections) are processed using blind deconvolution (10 iterations, NIS Elements, Nikon Inc.) to maximize information in each image and improve accuracy of subsequent object based analysis. Puncta (objects) are then segmented based on size and intensity using the 3D spot detection tool in NIS Elements General Analysis 3, which generates a binary layer containing spatial positioning of each object in x-, y- and z-axis for each individual protein label (i.e. 15LOX, PEBP1). Co-localization is tested using a Boolean “having” operation. This logical argument tests intersection of selected binary layers and identify objects that have both 15LOX/PEBP1.

Fluorescence assay of GSH. GSH levels are assessed using ThioGlo™-134 as described previously using a Shimadzu spectrofluorophotometer RF-5301PC (Shimadzu, Kyoto, Japan).

b) Regulatory mechanisms of AA-PE oxidation in TBI-induced neuronal death.

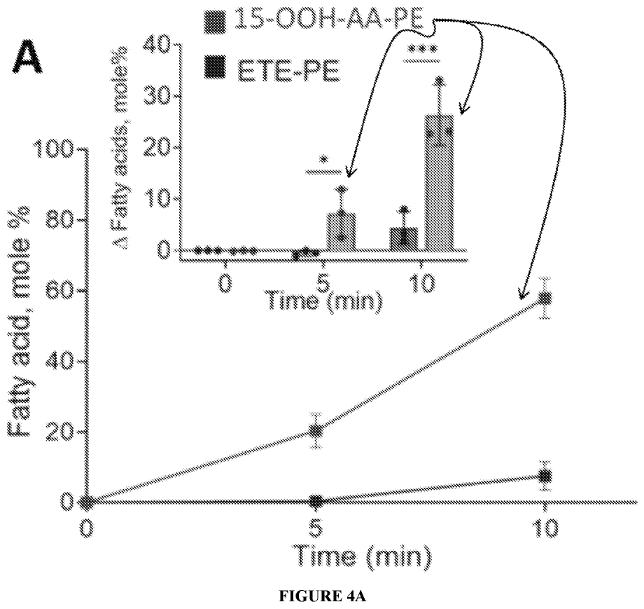

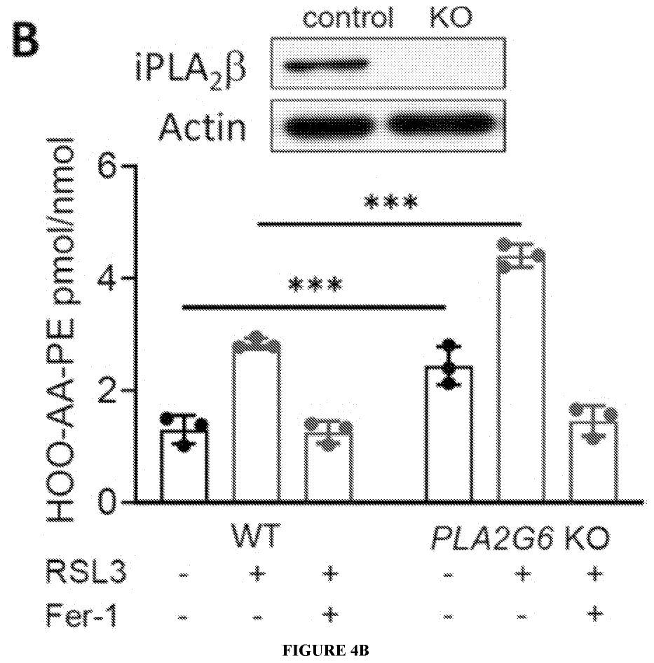

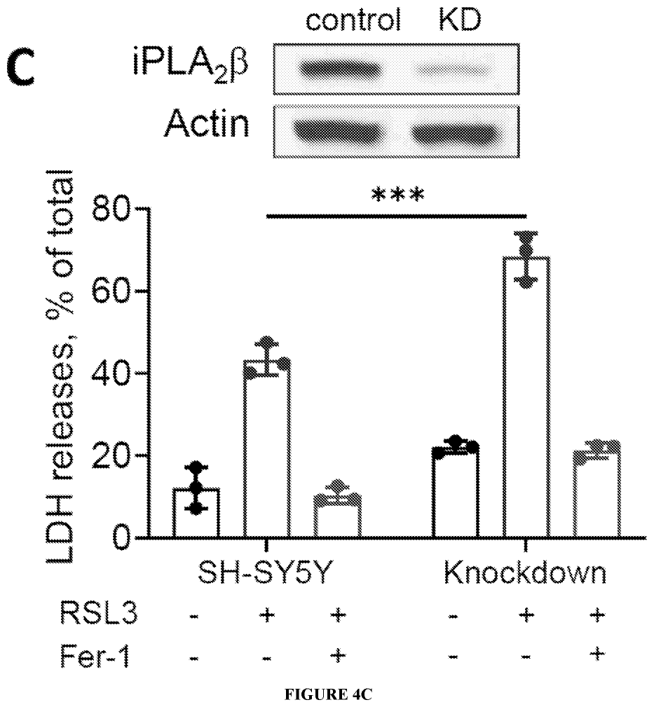

Experimental and methods: The effect of ACSL4, LPCAT3 and iPLA2β deficiency on PE oxidation and cell death in primary cortical neurons exposed to RSL3 and mechanical stretch is determined. siRNA approach is used to decrease ACSL4, LPCAT3 and iPLA2β protein levels. Pharmacological inhibitors including Triacsin C and rosiglitazone (for ACSL4), CI-976 and Thimerosal (for LPCAT3) and S-Bel (for iPLA2β) are used. PL oxidation, neuronal death is evaluated as described above. FIG. 4C shows successful siRNA KD of iPLA2β in neuronal SHSY-5Y cell line.

Parallel in vivo studies are performed to evaluate the effect of ACSL4 and LPCAT3 inhibition on PL and free fatty acid oxidation (particularly AA-PE, AA and AA-PE) as well as histological and functional outcome in PND17 rats after CCI. One inhibitor for ACSL4 and one for LPCAT3 are selected based on their performance in attenuating mechanical stretch-induced neuronal death. A comparison of two separate doses of selected inhibitors with vehicle is made in sham and CCI injured rats. The inhibitors are given i.v. within 1 h after injury. FIG. 3A-FIG. 3C shows that successful translation of compounds (e.g., Triacsin C) from mechanical stretch model to CCI in terms of dose and timing. To maximize rigor, rats are randomize to treatment groups; the investigators performing the surgery and outcome testing are blinded to treatment group. Sample size of 4-6/group should be sufficient to detect differences in biochemical studies based on work evaluating the effect of TBI on PL oxidation and treatment on markers of cell death and PL oxidation. Data are presented as mean±SD and analyzed using 1 or 2-factor ANOVA with Tukey's (or Dunn's test if not normally distributed) post-hoc test. Sample size estimates for histological and functional outcome are typically n=10 (required to detect a 30% difference between groups with an α=0.05 and power=0.8133-135). For histological outcome, comparisons between treatment groups are done using 2-way ANOVA with Tukey's post hoc test. For functional outcome, comparisons between treatment groups are done using 2-way RM-ANOVA with Bonferroni's post hoc test.

RNA interference. siRNA targeting rat ACSL4, LPCAT3 and iPLA2β and pooled scrambled control siRNA are purchased commercially. Primary neurons are transfected on day 4 with siRNA using Lipofectamine 2000 (Invitrogen). KD efficacy is assessed by WB. Timing of optimal KD is 72 h after transfection. Dose (25-100 nM) and time course (48-96 h) analysis of siRNA KD in normal neurons are performed first. Once optimal KD conditions are obtained, stretch injury and RSL3 exposure are performed.

Histological outcome assessment. Brains are quantitatively analyzed for total number of specific cell types and lesion volume using unbiased stereology as described. Diagnostic of ferroptosis in vivo are: 1) accumulation of doubly and triply oxygenated AA-PE species; 2) increased ACSL4; 3) decreased GPX4 activity or deficiency of GSH; 4) increased activity and expression of 15LOX; and 5) high levels of 15LOX-PEBP1 co-localization detectable by dual object recognition. Ferroptotic cells exhibit shrunken and damaged mitochondria without changes in nuclear morphology. TUNEL staining is used to quantify cell death. TUNEL is commonly used to detect apoptotic cells. However, cells undergoing other types of regulated death, including ferroptosis, are also TUNEL positive. In order to determine the apoptotic cell death, staining with cleaved caspase-3 antibody is performed.

Functional outcome assessment. Motor function is tested by beam-balance and inclined plane tasks.

Neurocognitive function is assessed by novel object recognition (NOR) and MWM (Morris Water Maze) tests. For NOR testing, rat is placed in a small enclosure with two similar objects and allowed to explore for a 35 min habituation phase. 24 h later (postoperative d10) rat is placed back in arena, where 1 of the original objects is replaced with a novel one. Percent time exploring original vs. novel object is recorded (2 min max). MWM test is done on post-operative d11 with a block of 4 daily trials (4-min inter-trial interval) for five consecutive days (d12-16) to locate hidden escape platform. Visible platform performance and memory retention in a single probe trial on d17 are assessed. Latency to find hidden platform and % time in target quadrant represent cognitive outcomes. Swim speed serves as a control to ensure there are no unrecognized motor deficits between groups.

c) Design and Investigate Mechanism of Action and Neuroprotective Potential of Small-Molecule Regulators of 15LOX and 15LOX/PEBP1 Activity in TBI.

There are several known general ferroptosis inhibitors, such as Fer-1 and baicalein. Fer-1 is an aromatic amine that acts as a general scavenger of lipid radicals and ROS. Fer-1 is widely used to confirm ferroptotic mechanism of cell death in vitro however its low bioavailability hinders in vivo use. Baicalein is a highly potent reductant with three hydroxy-groups in chromane ring that has a well-documented activity as a pan-LOX inhibitor. Baicalein has additional pharmacological activities associated with its ligand binding of benzodiazepine receptor as well as antiestrogen activity. This obscures mechanism of baicalein's action exclusively as suppression of 15LOX.

There is a substantial conceptual gap in current approaches to design 15LOX inhibitors. The major approach is based on known crystal structures of the enzyme to design orthosteric inhibitors that target catalytic site. Such inhibitors would competitively bind the pocket that would otherwise accommodate the major substrate—free AA159 and thus inhibit production of eicosanoid-, docosapentanoid-, and docosahexanoid-lipid mediators, particularly those with pro-resolving propensities. To avoid this undesirable interference with biosynthesis of important lipid mediators, specific inhibitors of 15LOX/PEBP1 complex were designed. Indeed, change in specificity of 15LOX to bind and oxygenate AA-PEs (rather than free AA, DPA and DHA) results from allosteric gain-in function acquired upon interaction with PEBP1.

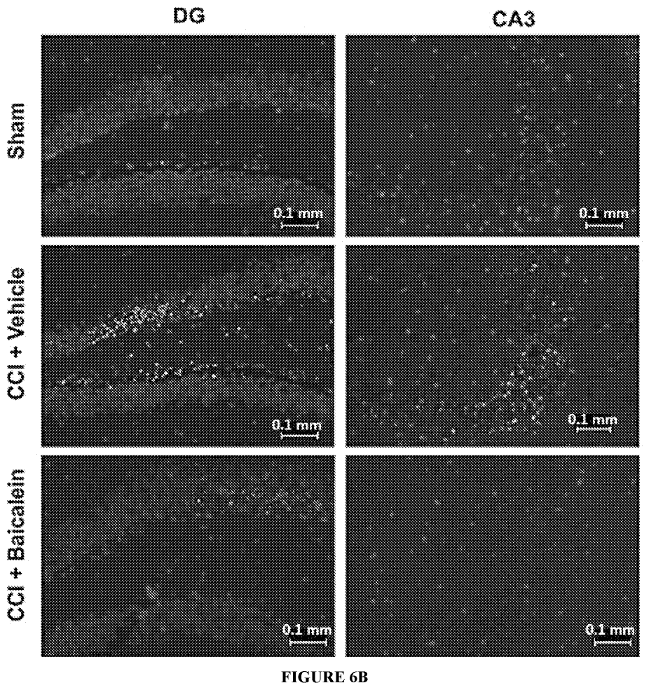

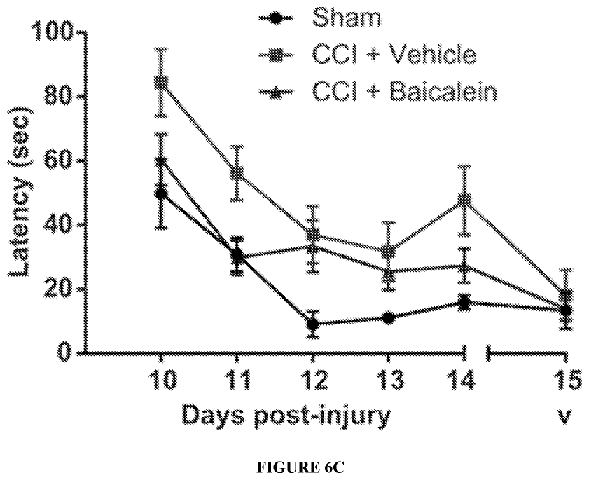

Preliminary results: In preliminary experiments, baicalein decreased AA-PE oxidation, attenuated hippocampal TUNEL positivity and functional deficits in MWM task when administered at a dose of 50 mg/kg i.p. 15 min after CCI (FIG. 5A, FIG. 5B, FIG. 6A-FIG. 6C). There is, however, room for improvement in the level of neuroprotection achieved. This may be related to the fact that as an effective general inhibitor of 15LOX, baicalein can suppress production of essential lipid mediators from free AA, DPA and DHA that may play important roles in memory as well as in anti-inflammatory processes.

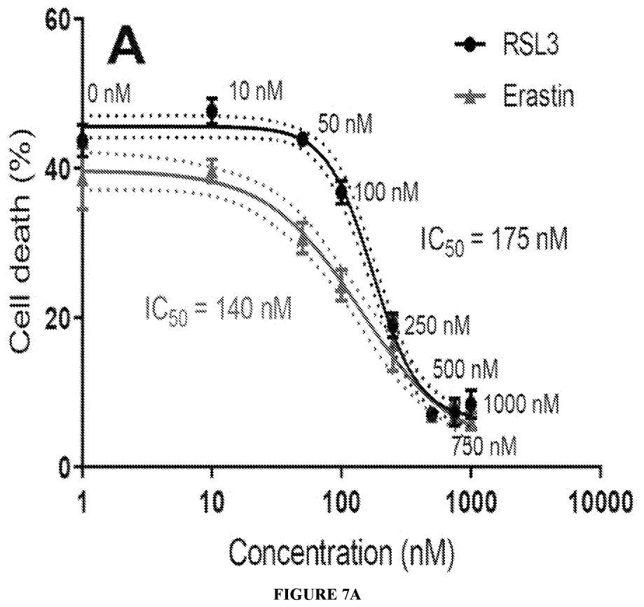

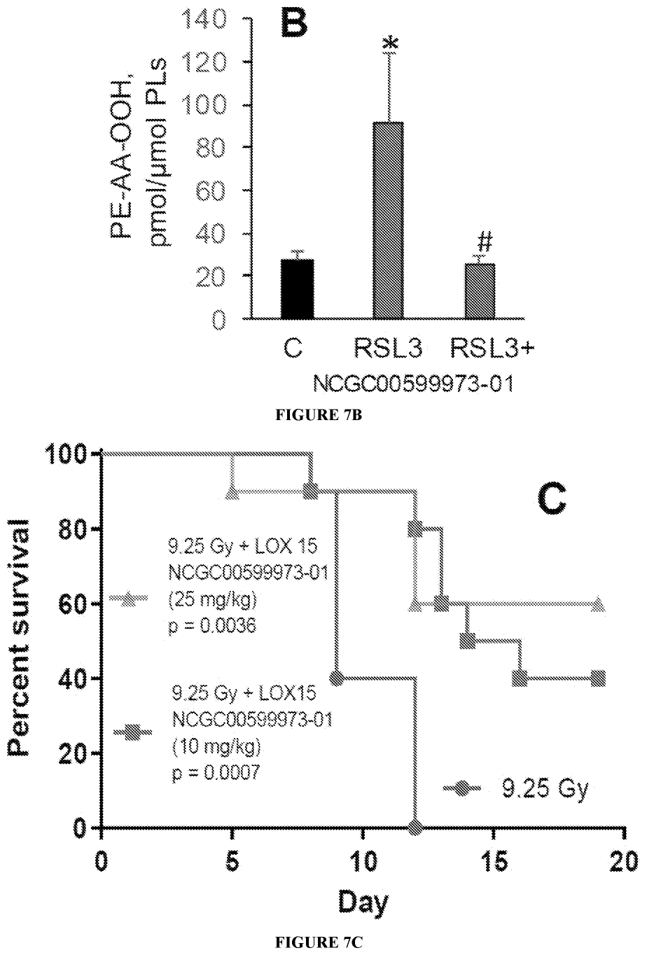

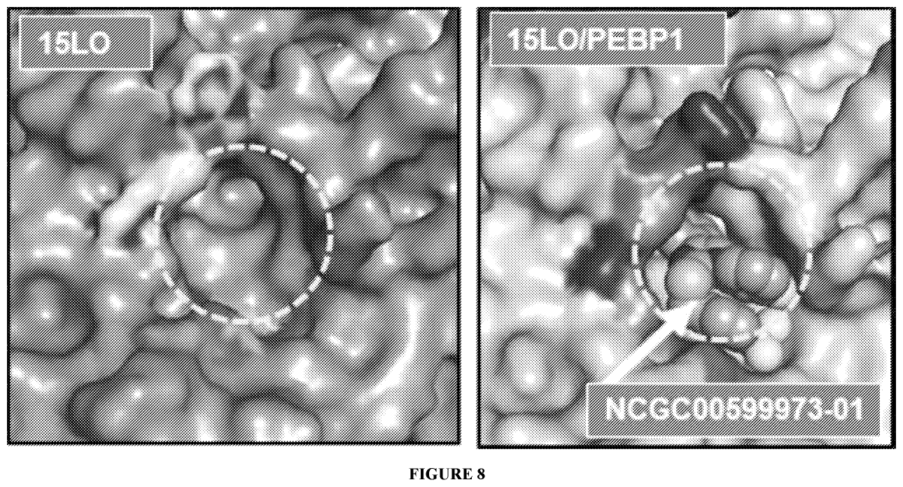

It was established that a change in substrate specificity of 15LOX is achieved upon complex formation with PEBP1 whereby structural rearrangements are mainly responsible for the selectivity and specificity leading to the formation of 15-HOO-AA-PE as the oxidation product. Thus, new selective anti-ferroptotic inhibitors of 15LOX/PEBP1 complex were designed. These inhibitors should not interfere with oxidation of free AA, DPA, DHA. Note that free OOH-AA does not represent a ferroptotic death signal. NCGC00599973-01 inhibits activity of 15LOX/PEBP1 complex. This compound suppressed ferroptosis triggered by RSL3 (GPX4 inhibition) or Erastin (GSH depletion) in cells with half maximal activity of 175 nM and 140 nM, respectively (FIG. 7A). The compound is a poor inhibitor of 15LOX alone with free AA as a substrate. Even at ˜1,000 times higher concentration (20 μM), inhibition of 15LOX alone was ˜4.5%. Preliminary docking studies of inhibitor on 15LOX/PEBP1 complex showed two interesting sites (FIG. 8) amongst top 5 docking poses from five different simulations. Site 1 is at 15LOX/PEBP1 binding interface (binding energy −10.1 kcal/mol) and site 2 is at catalytic site (binding energy −8.9 kcal/mol). Importantly, this inhibitor does not dock at catalytic site of 15LOX alone (FIG. 8). It was also shown that NCG-01 suppresses RSL3-induced accumulation of pro-ferroptotic HOO-AA-PE in vitro (FIG. 7B) and improves survival in a model of oxidant injury induced by ionizing radiation (FIG. 7C).

Experimental: to examine neuroprotective potential of 15LOX/PEBP1 inhibitor NCG-01 vs. baicalein and design 15LOX/PEBP1 inhibitors, a basic pharmacokinetic (PK) profile is established. This step helps to (i) determine dosing amount and (ii) highlight any serious adverse effects of NCG-01. Naïve PND17 male and female rats are treated with a single dose of inhibitor (10, 30, 60 mg/kg iv) or vehicle (n=5/grp). The relationship between plasma, liver and kidney levels of the compound over a 24 h period (at 10, 30, 60 min; 4 h, 12 h, 24 h, 72 h, 168 h) is analyzed using LC-MS. Calculation of the time to maximal plasma and liver tissue level (Tₘₐₓ) and the basic PK parameters (t_{1/2}, V_{d}, Cₘₐₓ, Cl) of the compound are determined. An assessment of CSF and brain levels of the compound using LC-MS and imaging MS is made. To determine whether NCG-01 induces toxicity, EKG (QT interval) plasma pH, glucose, albumin, AST/ALT/AlkP (liver enzymes), BUN, creatinine, and electrolytes over a 7-day period following single-dose administration using a commercial laboratory are performed.

Next, verification of biochemical target engagement of NCG-01 in brain is made and definition of optimal dosing regimens by assessing regional and cellular drug concentrations and inhibition of PE oxidation is done. PND 17 rats are randomized to receive NCG-01, baicalein or vehicle given i.v. 15 min after CCI or sham injury (n=5/grp). Rats are sacrificed at 4, 24, or 72 h, brains harvested, and cortical and hippocampal regions dissected on ice. A single i.v. dose 10, 25, 50 mg/kg NCG-01 and 50 mg/kg baicalein on PE oxidation (using LCMS) and 15LOX-PEP1 activity (with exogenous HOO-AA-PE as a substrate) is performed. In addition to PE oxidation, assessment of generation of pro- and anti-inflammatory lipid mediators from free fatty acids such as prostaglandins (PG) PGF2α, and PGD2, leukotriene B4 (LTB4), Lipoxin A4 (LXA4), resolvin D3 (RvD3), and neuroprotection D1 (NPD1) are made. The doses are based on data showing improved survival after oxidant injury (FIG. 7A-FIG. 7C) and improved functional outcome after CCI (FIG. 6A-FIG. 6C). Therapeutic window (15, 60, 120 min) is determined using optimal dosing regimen based on PD.

Sample sizes and data analysis: An initial group of n=5/sex/group is used. Thus, for each treatment (NCG-01, baicalein, vehicle), dose (0, 25, 50 mg/kg), time point (0, 4, 24, 72 h) and tissue need (homogenate, sections) 300 rats are needed. Based on these data therapeutic window studies are designed. If there are no sex-dependent PK/PD differences, a combination of sexes and reduction of sample sizes are made or increase in sample size if sex-dependent differences are observed but study is underpowered. For resource intensive studies, only the dosing and timepoint associated with maximal AA-PE-OOH inhibition are used.

Preclinical trial comparing NCG-01 and baicalein. Rats are randomized into sham vehicle, sham drug, CCI vehicle, or CCI drug groups. Dose timing are based on previous results above. Rats undergo motor and neurocognitive function and histological assessment as described above. Safety: Drug treated sham and CCI rats are monitored for physiological derangements and undergo all biochemical, behavioral and histological outcomes.

Sample sizes and data analysis: An initial plan to use an n=10/group/treatment/sex=200 is made. 3 dosing regimens of NCG-01 are utilized, 1 dosing regimen for baicalein and vehicle in PND17 rats. Sample sizes for histological and functional outcome are based on studies in the MWM testing in PND17 rats after CCI15. For α=0.05 and power=0.8 an n=10 is required if shams are combined and sexes are analyzed separately. If no differences are detected between male and female rats within groups, they are combined to increase statistical power. The smallest sample size possible is used that allows for reliable data interpretation. For histological outcome, comparisons between treatment groups are done using 2-way ANOVA with Tukey's post hoc test. For functional outcome, comparisons between treatment groups are done using 2-way RM-ANOVA with Bonferroni's post hoc test.

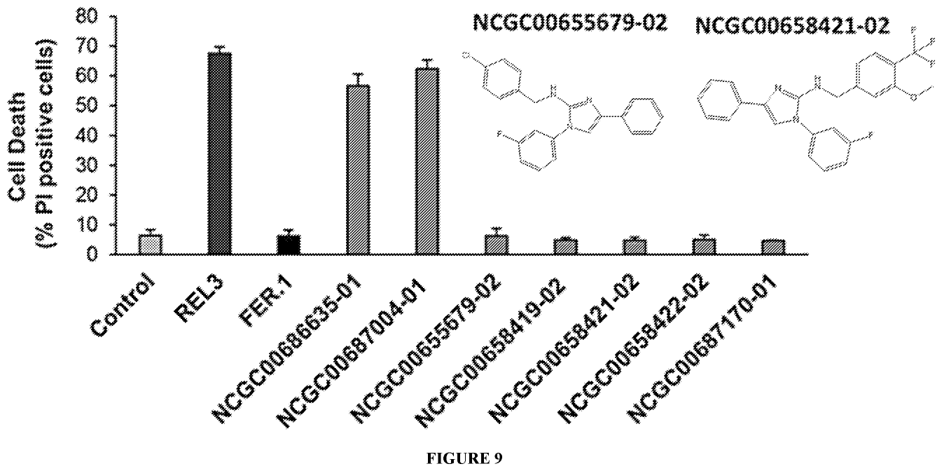

Development and testing of new compounds that inhibit 15LOX/PEBP1 complex activity. A combination of computational and experimental studies are used. New molecules that selectively suppress pro-ferroptotic HOO-AA-PE formation from AA-PE by 15LOX/PEBP1 complex but do not suppress 15LOX's catalytic activity towards the canonical substrate, free AA are developed. Computer modeling considers specific features of organization of 15LOX/PEBP1 complexes and positioning of AA-PE as well as regio-specific oxygenation of AA-residues at 15^{th} carbon. This is essential as only 15-HOO-AA-PE represents the pro-ferroptotic signals, while oxygenated free HOO-AA—does not. It has been shown that: i) binding interface of 15LOX in 15LOX/PEBP1 complex includes a helix on 15LOX (α2) and kinase binding epitope (D144, H145, R146) on PEBP1 (FIG. 8); and ii) interfacial interactions play a pivotal role in allosterically altering conformational dynamics of 15LOX in complex, and its substrate specificity. Analysis of change in access to catalytic site upon PEBP1 binding, using elastic network models indicates needed sites at 15LOX/PEBP1 binding interface. Dynamics simulations are also performed, for 15LOX alone and 15LOX/PEBP1 complex. An effective inhibitor, NCG-01, with in vivo activity has been indicated. Several other 15LOX/PEBP1 inhibitors with anti-ferroptotic effect (FIG. 9) have been identified. To test the effectiveness of compounds identified from the computational screen, RSL3 and mechanical stretch models with LDH and PI assays as a primary outcome are performed. The compounds that inhibit cell death by at least 50% are then evaluated for their effects on AA-PE and AA oxidation by 15LOX/PEBP1 complex and 15LOX alone, respectively, by LC-MS. Evaluation of the effect of compounds in suppressing AA-PE-OOH formation in cells upon proferroptotic stimuli is also performed.

### Example 2: Synthesis of 15LOX/PEBP1 Complex Inhibitory Compounds and Potency

The present disclosure encompasses compounds for use in the inhibition of 15LOX/PEBP1 complex. The compounds may have the formula below:

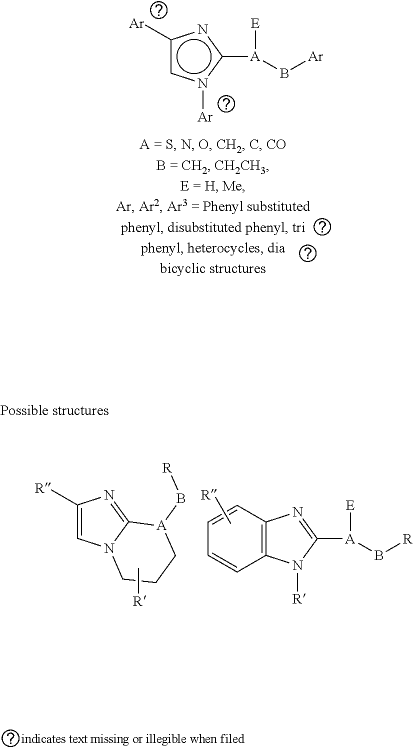

General Methods for Chemistry. All air or moisture sensitive reactions were performed under positive pressure of nitrogen with oven-dried glassware. Chemical reagents and anhydrous solvents were obtained from commercial sources and used as-is. Preparative purification was performed on a Waters semi-preparative HPLC. The column used was a Phenomenex Luna C18 (5-micron, 30×75 mm) at a flow rate of 45 mL/min. The mobile phase consisted of acetonitrile and water (each containing 0.1% trifluoroacetic acid). A gradient of 10% to 50% acetonitrile over 8 minutes was used during the purification. Fraction collection was triggered by mass detection. Analytical analysis for purity was determined by two different methods denoted as Final QC Methods 1 and 2. Method 1: Analysis was performed on an Agilent 1290 Infinity Series HPLC. HPLC Short Gradient (Method 1) 4% to 100% acetonitrile (0.05% trifluoroacetic acid) in water over 3.5 minutes run time of 4 minutes with a flow rate of 0.8 mL/min. A Phenomenex Kinetex 1.7 micron C18 column (2.1×100 mm) was used at a temperature of 50° C. Method 2: analysis was performed on an Agilent 1290 with a 7 minute gradient of 4% to 100% acetonitrile (containing 0.025% trifluoroacetic acid) in water (containing 0.05% trifluoroacetic acid) over 8 minute run time at a flow rate of 1 m/min. A Phenomenex Luna C18 column (3-micron, 3×75 mm) was used at a temperature of 50° C. Purity determination was performed using an Agilent Diode Array Detector for both Method 1 and Method 2. Mass determination was performed using an Agilent 6130 mass spectrometer with electrospray ionization in the positive mode. All the analogs for assay have purity greater than 95% based on both analytical methods. ¹H and ¹³C NMR spectra were recorded on a Varian 400 (100) MHz spectrometer. High resolution mass spectrometry was recorded on Agilent 6210 Time-of-Flight LC/MS system.

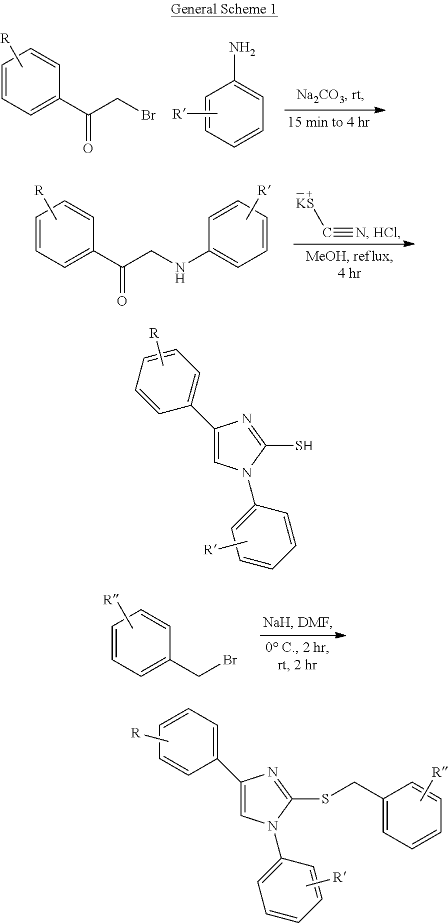

General Method 1: 2-bromo-1-phenylethan-1-one (1.01 mmol), aniline (1.11 mmol), and Na₂CO₃ (1.01 mmol) are mortared for 3 min let sit in the mortar for 15 min to 4 hr. at rt (dependent on aniline). The solid is rinsed with water and then washed with MeOH into a round bottom flask. The resulting mixture is dried to a pale-yellow powder, crystalized with MeOH, and filtered to recover a white solid with a yield of 58% to 83% and purity of 95% by LCMS.

A slurry of 2-((phenyl)amino)-1-phenylethan-1-one (0.44 mmol) in MeOH (2.20 mL) is stirred and treated with potassium thiocyanate (0.87 mmol) and concentrated HCl (0.44 mmol) followed by heating to reflux for 4 h. After cooling to room temperature, the solids are filtered to give a white solid at 95% pure by LCMS in a 99% yield. The white solid is used as is in the next reaction.

1,4-diphenyl-1H-imidazole-2-thiol (0.44 mmol) in DMF (2.20 mL) is cooled to 0° C. in an ice bath for 15 min before adding NaH (0.53 mmol) and stirred for 2 hours in the ice bath. 1-(bromomethyl)-4-(trifluoromethyl)benzene (0.44 mmol) is added to the reaction mixture at 0° C. and allowed to stir at rt for 2 hr. The reaction is quenched by chilling the solution to ice bath temp and adding drops of MeOH and water. This mixture is placed on a reverse phase waters and purified to give the desired product.

1,4-Diphenyl-2-((4-(trifluoromethyl)benzyl)thio)-1H-imidazole (DKL040-009, NCGC00600066): ¹H NMR (400 MHz, DMSO-d₆) δ 7.98 (s, 1H), 7.86-7.78 (m, 2H), 7.64-7.57 (m, 2H), 7.54-7.43 (m, 5H), 7.42-7.29 (m, 4H), 7.27-7.19 (m, 1H), 4.42 (s, 2H); LC-MS retention time (Method 1): 3.712 min; (Method 2): 6.407 min; HRMS: m/z (M+H)⁺=(Calculated for C₂₃H₁₈F₃N₂S, 411.1137) found, 411.1142.

1-(3-Chlorophenyl)-4-phenyl-2-((4-(trifluoromethyl)benzyl)thio)-1H-imidazole (DKL040-077, NCGC00600001): Follow General Method 1 using 3-chloroaniline ¹H NMR (400 MHz, DMSO-d₆) δ 8.04 (s, 1H), 7.86-7.80 (m, 2H), 7.59 (d, J=8.1 Hz, 2H), 7.55-7.49 (m, 2H), 7.47 (d, J=8.1 Hz, 2H), 7.42-7.35 (m, 3H), 7.35-7.29 (m, 1H), 7.28-7.22 (m, 1H), 4.40 (s, 2H); LC-MS retention time (Method 1): 3.869 min; (Method 2): 6.934 min; HRMS: m/z (M+H)⁺=(Calculated for C₂₃H₁₇ClF₃N₂S, 445.0748) found, 445.0753.

1-(2-Fluorophenyl)-4-phenyl-2-((4-(trifluoromethyl)benzyl)thio)-1H-imidazole (DKL039-098, NCGC00599992): Follow General Method 1 using 2-fluoroaniline: ¹H NMR (400 MHz, DMSO-d₆) δ 7.92 (d, J=0.8 Hz, 1H), 7.85-7.79 (m, 2H), 7.61-7.49 (m, 2H), 7.48-7.34 (m, 6H), 7.32-7.19 (m, 3H), 4.35 (s, 2H); LC-MS retention time (Method 1): 3.829 min; (Method 2): 6.871 min; HRMS: m/z (M+H)⁺=(Calculated for C₂₃H₁₇F₄N₂S 429.1043) found, 429.1039.

1-(3-Fluorophenyl)-4-phenyl-2-((4-(trifluoromethyl)benzyl)thio)-1H-imidazole (DKL039-097, NCGC00599973): Follow General Method 1 using 3-fluoroaniline: ¹H NMR (400 MHz, DMSO-d₆) δ 8.02 (s, 1H), 7.86-7.79 (m, 2H), 7.59 (d, J=8.1 Hz, 2H), 7.56-7.50 (m, 1H), 7.48 (d, J=8.2 Hz, 2H), 7.39 (t, J=7.7 Hz, 2H), 7.34-7.22 (m, 2H), 7.26-7.16 (m, 2H), 4.41 (s, 2H); LC-MS retention time (Method 1): 3.807 min; (Method 2): 6.888 min; HRMS: m/z (M+H)⁺=(Calculated for C₂₃H₁₇F₄N₂S 429.1054) found, 429.1061.

1-(4-Fluorophenyl)-4-phenyl-2-((4-(trifluoromethyl)benzyl)thio)-1H-imidazole (DKL039-096, NCGC00599971): Follow General Method 1 using 4-fluoroaniline: ¹H NMR (400 MHz, DMSO-d₆) δ 7.98 (s, 1H), 7.85-7.78 (m, 2H), 7.60 (d, J=8.1 Hz, 2H), 7.49 (d, J=8.0 Hz, 2H), 7.47-7.34 (m, 4H), 7.37-7.27 (m, 2H), 7.27-7.19 (m, 1H), 4.40 (s, 2H); LC-MS retention time (Method 1): 3.780 min; (Method 2): 6.701 min; HRMS: m/z (M+H)⁺=(Calculated for C₂₃H₁₇F₄N₂S 429.1043) found, 429.1045.

1-(4-Chlorophenyl)-4-phenyl-2-((4-(trifluoromethyl)benzyl)thio)-1H-imidazole (DKL039-099, NCGC00599991): Follow General Method 1 using 4-chloroaniline ¹H NMR (400 MHz, DMSO-d₆) δ 8.00 (s, 1H), 7.85-7.78 (m, 2H), 7.63-7.51 (m, 4H), 7.51-7.45 (m, 2H), 7.44-7.31 (m, 4H), 7.28-7.20 (m, 1H), 4.41 (s, 2H); LC-MS retention time (Method 1): 3.852 min; (Method 2): 7.069 min; HRMS: m/z (M+H)⁺=(Calculated for C₂₃H₁₇ClF₃N₂S 445.0748) found, 445.0744.

4-Phenyl-1-(p-tolyl)-2-((4-(trifluoromethyl)benzyl)thio)-1H-imidazole (DKL040-001, NCGC00599997): Follow General Method 1 using 4-methylaniline: ¹H NMR (400 MHz, DMSO-d₆) δ 7.94 (s, 1H), 7.84-7.77 (m, 2H), 7.60 (d, J=8.1 Hz, 2H), 7.51 (d, J=8.1 Hz, 2H), 7.38 (t, J=7.7 Hz, 2H), 7.31-7.14 (m, 5H), 4.41 (s, 2H), 2.34 (s, 3H); LC-MS retention time (Method 1): 3.753 min; (Method 2): 6.754 min; HRMS: m/z (M+H)⁺=(Calculated for C₂₄H₂₀F₃N₂S 425.1294) found, 425.1313.

2-(Benzylthio)-1-phenyl-4-(4-(trifluoromethyl)phenyl)-1H-imidazole (DKL039-058, NCGC00599877): Reflux 2-(phenylamino)-1-(4-(trifluoromethyl)phenyl)ethan-1-one (50.0 mg 0.18 mmol), and (thiocyanatomethyl)benzene (24 mg, 0.16 mmol), in DMF (1 mL) for 1 hr. The reaction was followed by LCMS to completion. The material was cooled to room temperature and turned in for reverse phase purification with no workup ¹H NMR (400 MHz, DMSO-d₆) δ 8.19 (s, 1H), 8.07-8.02 (m, 2H), 7.76-7.72 (m, 2H), 7.55-7.43 (m, 3H), 7.41-7.34 (m, 2H), 7.31 (ddt, J=5.0, 3.5, 1.4 Hz, 2H), 7.28-7.18 (m, 3H), 4.38 (s, 2H); LC-MS retention time (Method 1): 3.792 min; (Method 2): 6.977 min; HRMS: m/z (M+H)⁺=(Calculated for C₂₃H₁₈F₃N₂S 411.1137) found, 411.1135.

1-(3,5-Difluorophenyl)-4-phenyl-2-((4-(trifluoromethyl)benzyl)thio)-1H-imidazole (DKL040-079, NCGC00600614): Follow General Method 1 using 3,5-difluoroaniline. ¹H NMR (400 MHz, DMSO-d₆) δ 8.05 (s, 1H), 7.85-7.78 (m, 2H), 7.61-7.55 (m, 2H), 7.47-7.32 (m, 5H), 7.30-7.21 (m, 1H), 7.19-7.08 (m, 2H), 4.38 (s, 2H). LC-MS retention time (Method 1): 3.862 min; (Method 2): 6.913 min; HRMS: m/z (M+H)⁺=(Calculated for C₂₃H₁₆F₅N₂S 447.0949) found, 447.0956.

1-Phenyl-2-((4-(trifluoromethyl)benzyl)thio)-4-(4-(trifluoromethyl)phenyl)-1H-imidazole (DKL039-082, NCGC00599898): Follow General Method 1 starting with 2-bromo-1-(4-(trifluoromethyl)phenyl)ethan-1-one: ¹H NMR (400 MHz, DMSO-d₆) δ 8.20 (s, 1H), 8.04 (d, J=8.1 Hz, 2H), 7.74 (d, J=8.3 Hz, 2H), 7.61 (d, J=8.1 Hz, 2H), 7.57-7.41 (m, 5H), 7.39-7.28 (m, 2H), 4.45 (s, 2H); LC-MS retention time (Method 1): 3.716 min; (Method 2): 7.281 min; HRMS: m/z (M+H)⁺=(Calculated for C₂₄H₁₇F₆N₂S 479.1011) found, 479.1004.

4-Phenyl-2-((4-(trifluoromethyl)benzyl)thio)-1-(3-(trifluoromethyl)phenyl)-1H-imidazole (DKL040-078, NCGC00600653): Follow General Method 1 using 3-trifluoromethylaniline. LC-MS retention time (Method 1): 3.840 min; (Method 2): 6.979 min; HRMS: m/z (M+H)⁺=(Calculated for C₂₄H₁₇F₆N₂S 479.1011) found, 479.1027.

1-(3-Bromophenyl)-4-phenyl-2-((4-(trifluoromethyl)benzyl)thio)-1H-imidazole (DKL040-082, NCGC00600636): Follow General Method 1 using 3-bromoaniline. ¹H NMR (400 MHz, DMSO-d₆) δ 8.04 (s, 1H), 7.86-7.80 (m, 2H), 7.65 (ddd, J=8.0, 1.9, 1.1 Hz, 1H), 7.60 (d, J=8.1 Hz, 2H), 7.53-7.43 (m, 4H), 7.43-7.33 (m, 3H), 7.29-7.18 (m, 1H), 4.41 (s, 2H); LC-MS retention time (Method 1): 3.890 min; (Method 2): 7.030 min; HRMS: m/z (M+H)+=(Calculated for C₂₃H₁₇BrF₃N₂S 491.0226) found, 491.0224.

4-Phenyl-1-(m-tolyl)-2-((4-(trifluoromethyl)benzyl)thio)-1H-imidazole (DKL040-080, NCGC00600630): Follow General Method 1 using 3-methylaniline. ¹H NMR (400 MHz, DMSO-d₆) δ 7.97 (s, 1H), 7.86-7.79 (m, 2H), 7.64-7.58 (m, 2H), 7.50 (d, J=8.1 Hz, 2H), 7.37 (q, J=7.6 Hz, 3H), 7.29-7.19 (m, 2H), 7.12 (ddd, J=7.8, 2.1, 1.1 Hz, 1H), 7.06 (t, J=1.5 Hz, 1H), 4.42 (s, 2H), 2.32 (s, 3H); LC-MS retention time (Method 1): 3.774 min; (Method 2): 6.613 min; HRMS: m/z (M+H)⁺=(Calculated for C₂₄H₂₀F₃N₂S 425.1294) found, 425.1298.

1-(3-(Methylsulfonyl)phenyl)-4-phenyl-2-((4-(trifluoromethyl)benzyl)thio)-1H-imidazole, (DKL040-081, NCGC00600635): Follow General Method 1 using 3-(methylsulfonyl)aniline. ¹H NMR (400 MHz, DMSO-d₆) δ 8.13 (s, 1H), 8.02-7.92 (m, 2H), 7.87-7.80 (m, 2H), 7.77 (t, J=7.8 Hz, 1H), 7.71 (ddd, J=8.0, 2.2, 1.2 Hz, 1H), 7.60 (d, J=8.2 Hz, 2H), 7.51 (d, J=8.1 Hz, 2H), 7.40 (t, J=7.7 Hz, 2H), 7.30-7.21 (m, 1H), 4.44 (s, 2H), 3.28 (s, 3H); LC-MS retention time (Method 1): 3.556 min; (Method 2): 6.072 min; HRMS: m/z (M+H)⁺=(Calculated for C₂₄H₂₀F₃N₂O₂S₂ 489.0913) found, 489.0912.

1-(3-Fluorophenyl)-4-phenyl-2-((4-(3-(trifluoromethyl)-3H-diazirin-3-yl)benzyl)thio)-1H-imidazole (DKL041-004, NCGC00600835): Follow General Method 1 using 3-fluoroaniline and 3-(4-(bromomethyl)phenyl)-3-(trifluoromethyl)-3H-diazirine. ¹H NMR (400 MHz, DMSO-d₆) δ 8.02 (s, 1H), 7.88-7.76 (m, 2H), 7.52 (td, J=8.2, 6.5 Hz, 1H), 7.44-7.34 (m, 4H), 7.33-7.06 (m, 6H), 4.35 (s, 2H); LC-MS retention time (Method 1): 3.903 min; (Method 2): 7.065 min; HRMS: m/z (M+H)⁺=(Calculated for C₂₄H₁₇F₄N₄S 469.1105) found, 469.1111.

2-(Benzylthio)-4-(4-bromophenyl)-1-(3-fluorophenyl)-1H-imidazole (DKL041-012, NCGC00600840): Follow General Method 1 using 2-bromo-1-(4-bromophenyl)ethan-1-one and 3-fluoroaniline in the first step, and (bromomethyl)benzene in the final step. ¹H NMR (400 MHz, DMSO-d₆) δ 8.09 (s, 1H), 7.82-7.74 (m, 2H), 7.61-7.55 (m, 2H), 7.55-7.50 (m, 1H), 7.35-7.16 (m, 8H), 4.35 (s, 2H); LC-MS retention time (Method 1): 3.904 min; (Method 2): 6.940 min; HRMS: m/z (M+H)⁺=(Calculated for C₂₂H₁₇BrFN₂S 441.0255) found, 441.0260.

1-(3-Methoxyphenyl)-4-phenyl-2-((4-(trifluoromethyl)benzyl)thio)-1H-imidazole (DKL041-020, NCGC00600960): Follow General Method 1 using 3-methoxyaniline. ¹H NMR (400 MHz, DMSO-d₆) δ 8.00 (s, 1H), 7.86-7.78 (m, 2H), 7.61 (d, J=8.1 Hz, 2H), 7.53 (d, J=8.1 Hz, 2H), 7.39 (td, J=8.1, 3.4 Hz, 3H), 7.28-7.19 (m, 1H), 7.01 (ddd, J=8.4, 2.4, 1.1 Hz, 1H), 6.94-6.87 (m, 2H), 4.44 (s, 2H), 3.77 (s, 3H); LC-MS retention time (Method 1): 3.721 min; (Method 2): 6.433 min; HRMS: m/z (M+H)⁺=(Calculated for C₂₄H₂₀F₃N₂OS 441.1243) found, 441.1259.

1-(3-(Difluoromethoxy)phenyl)-4-phenyl-2-((4-(trifluoromethyl)benzyl)thio)-1H-imidazole (DKL041-021, NCGC00600959): Follow General Method 1 using 3-difluoromethoxyaniline. ¹H NMR (400 MHz, DMSO-d₆) δ 8.03 (s, 1H), 7.86-7.79 (m, 2H), 7.60 (d, J=8.1 Hz, 2H), 7.54-7.47 (m, 3H), 7.39 (t, J=7.7 Hz, 2H), 7.29-7.21 (m, 4H), 7.18 (t, J=2.2 Hz, 1H), 4.43 (s, 2H); LC-MS retention time (Method 1): 3.757 min; (Method 2): 6.577 min; HRMS: m/z (M+H)⁺=(Calculated for C₂₄H₁₈F₅N₂OS 477.1055) found, 477.1075.

4-Phenyl-1-(3-(trifluoromethoxy)phenyl)-2-((4-(trifluoromethyl)benzyl)thio)-1H-imidazole (DKL041-026, NCGC00600973): Follow General Method 1 using 3-trifluoromethoxyaniline. ¹H NMR (400 MHz, DMSO-d₆) δ 8.06 (s, 1H), 7.87-7.79 (m, 2H), 7.65-7.55 (m, 3H), 7.50-7.35 (m, 6H), 7.31-7.20 (m, 2H), 4.39 (s, 2H); LC-MS retention time (Method 1): 3.891 min; (Method 2):7.006 min; HRMS: m/z (M+H)⁺=(Calculated for C₂₄H₁₇F₆N₂OS 495.0960) found, 495.0947.

1-(3-Fluorophenyl)-2-((4-methylbenzyl)thio)-4-phenyl-1H-imidazole(DKL041-046, NCGC00601881): Follow General Method 1 using 3-fluoroaniline in the first step, and 1-(bromomethyl)-4-methylbenzene in the final step. ¹H NMR (400 MHz, DMSO-d₆) δ 8.02 (s, 1H), 7.87-7.80 (m, 2H), 7.54 (td, J=8.3, 6.5 Hz, 1H), 7.39 (t, J=7.7 Hz, 2H), 7.35-7.26 (m, 1H), 7.29-7.18 (m, 3H), 7.18-7.11 (m, 2H), 7.04 (d, J=7.8 Hz, 2H), 4.30 (s, 2H), 2.23 (s, 3H); LC-MS retention time (Method 1): 3.181 min; (Method 2): 6.297 min; HRMS: m/z (M+H)+=(Calculated for C₂₃H₂₀FN₂S 375.1326) found, 375.1324.

1-(3-Fluorophenyl)-2-((4-(methylsulfonyl)benzyl)thio)-4-phenyl-1H-imidazole (DKL041-049, NCGC00601948): Follow General Method 1 using 3-fluoroaniline in the first step, and 1-(bromomethyl)-4-(methylsulfonyl)benzene in the final step. ¹H NMR (400 MHz, DMSO-d₆) δ 8.04 (s, 1H), 7.81 (ddd, J=13.4, 7.5, 1.6 Hz, 4H), 7.60-7.49 (m, 3H), 7.44-7.18 (m, 6H), 4.45 (s, 2H), 3.16 (s, 3H); LC-MS retention time (Method 1): 3.329 min; (Method 2): 5.470 min; HRMS: m/z (M+H)⁺=(Calculated for C₂₃H₂₀FN₂O₂S₂ 439.0945) found, 439.0955.

2-((4-Chlorobenzyl)thio)-1-(3-fluorophenyl)-4-phenyl-1H-imidazole (DKL041-048, NCGC00601951): Follow General Method 1 using 3-fluoroaniline in the first step, and 1-(bromomethyl)-4-chlorobenzene in the final step. ¹H NMR (400 MHz, DMSO-d₆) δ 8.03 (s, 1H), 7.86-7.79 (m, 2H), 7.55 (td, J=8.2, 6.4 Hz, 1H), 7.39 (t, J=7.7 Hz, 2H), 7.35-7.19 (m, 8H), 4.33 (s, 2H); LC-MS retention time (Method 1): 3.723 min; (Method 2): 6.472 min; HRMS: m/z (M+H)⁺=(Calculated for C₂₂H₁₇ClFN₂S 395.0780) found, 395.0780.

2-((4-Bromobenzyl)thio)-1-(3-fluorophenyl)-4-phenyl-1H-imidazole (DKL041-050, NCGC00601930): Follow General Method 1 using 3-fluoroaniline in the first step, and 1-(bromomethyl)-4-bromobenzene in the final step. ¹H NMR (400 MHz, DMSO-d₆) δ 8.03 (s, 1H), 7.86-7.79 (m, 2H), 7.54 (td, J=8.2, 6.4 Hz, 1H), 7.47-7.19 (m, 10H), 4.32 (s, 2H); LC-MS retention time (Method 1): 3.786 min; (Method 2): 6.562 min; HRMS: m/z (M+H)⁺=(Calculated for C₂₂H₁₇BrFN₂S 441.0255) found, 441.0262.

1-(3-Fluorophenyl)-4-phenyl-2-((3-(trifluoromethyl)benzyl)thio)-1H-imidazole (DKL041-047, NCGC00601931): Follow General Method 1 using 3-fluoroaniline in the first step, and 1-(bromomethyl)-3-trifluoromethylbenzene in the final step. ¹H NMR (400 MHz, DMSO-d₆) δ 8.02 (s, 1H), 7.87-7.79 (m, 2H), 7.70 (s, 1H), 7.61-7.42 (m, 4H), 7.38 (t, J=7.7 Hz, 2H), 7.31 (td, J=8.5, 2.5 Hz, 1H), 7.26-7.12 (m, 3H), 4.41 (s, 2H); LC-MS retention time (Method 1): 3.780 min; (Method 2): 6.573 min; HRMS: m/z (M+H)⁺=(Calculated for C₂₃H₁₇F₄N₂S 429.1043) found, 429.1023.

1-(3-Fluorophenyl)-4-phenyl-2-((1-phenylethyl)thio)-1H-imidazole (DKL041-057, NCGC00601944): Follow General Method 1 using 3-fluoroaniline in the first step, and (1-bromoethyl)benzene in the final step. ¹H NMR (400 MHz, DMSO-d₆) δ 8.01 (s, 1H), 7.87-7.80 (m, 2H), 7.52 (td, J=8.3, 6.4 Hz, 1H), 7.39 (t, J=7.7 Hz, 2H), 7.34-7.19 (m, 7H), 7.16 (ddd, J=8.0, 5.8, 2.3 Hz, 2H), 4.71 (q, J=7.0 Hz, 1H), 1.63 (d, J=7.0 Hz, 3H); LC-MS retention time (Method 1): 3.614 min; (Method 2): 6.141 min; HRMS: m/z (M+H)⁺=(Calculated for C₂₃H₂₀FN₂S 375.1326) found, 375.1332.

2-(Benzylthio)-1-(3-fluorophenyl)-4-phenyl-1H-imidazole (DKL041-056, NCGC00601943): Follow General Method 1 using 3-fluoroaniline in the first step, and (bromomethyl)benzene in the final step. ¹H NMR (400 MHz, DMSO-d₆) δ 8.03 (s, 1H), 7.89-7.78 (m, 2H), 7.54 (td, J=8.1, 6.4 Hz, 1H), 7.39 (t, J=7.7 Hz, 2H), 7.36-7.14 (m, 9H), 4.35 (s, 2H); LC-MS retention time (Method 1): 3.624 min; (Method 2): 6.074 min; HRMS: m/z (M+H)⁺=(Calculated for C₂₂H₁₈FN₂S 361.1169) found, 361.1170.

2-((3-Chlorobenzyl)thio)-1-(3-fluorophenyl)-4-phenyl-1H-imidazole (DKL041-054, NCGC00601947): Follow General Method 1 using 3-fluoroaniline in the first step, and 1-(bromomethyl)-3-chlorobenzene in the final step. ¹H NMR (400 MHz, DMSO-d₆) δ 8.03 (s, 1H), 7.88-7.79 (m, 2H), 7.55 (td, J=8.2, 6.4 Hz, 1H), 7.44-7.36 (m, 3H), 7.35-7.18 (m, 7H), 4.33 (s, 2H); LC-MS retention time (Method 1): 3.763 min; (Method 2): 6.505 min; HRMS: m/z (M+H)⁺=(Calculated for C₂₂H₁₇ClFN₂S 395.0780) found, 395.0782.

1-(3-Fluorophenyl)-4-phenyl-2-((4-(trifluoromethoxy)benzyl)thio)-1H-imidazole (DKL041-053, NCGC00601942): Follow General Method 1 using 3-fluoroaniline in the first step, and 1-(bromomethyl)-4-(trifluoromethoxy)benzene in the final step. ¹H NMR (400 MHz, DMSO-d₆) δ 8.04 (s, 1H), 7.86-7.79 (m, 2H), 7.53 (td, J=8.2, 6.4 Hz, 1H), 7.39 (dd, J=8.4, 6.9 Hz, 4H), 7.35-7.17 (m, 6H), 4.37 (s, 2H); LC-MS retention time (Method 1): 3.800 min; (Method 2): 6.666 min; HRMS: m/z (M+H)⁺=(Calculated for C₂₃H₁₇F₄N₂OS 445.1005) found, 445.1003.

1-(3-Fluorophenyl)-2-((3-methylbenzyl)thio)-4-phenyl-1H-imidazole (DKL041-055, NCGC00601955): Follow General Method 1 using 3-fluoroaniline in the first step, and 1-(bromomethyl)-3-methylbenzene in the final step. ¹H NMR (400 MHz, DMSO-d₆) δ 8.02 (s, 1H), 7.88-7.81 (m, 2H), 7.54 (td, J=8.4, 6.5 Hz, 1H), 7.39 (t, J=7.7 Hz, 2H), 7.37-7.16 (m, 4H), 7.15-6.99 (m, 4H), 4.29 (s, 2H), 2.21 (s, 3H); LC-MS retention time (Method 1): 3.718 min; (Method 2): 6.254 min; HRMS: m/z (M+H)⁺=(Calculated for C₂₃H₂₀FN₂S 375.1326) found, 375.1338.

1-(3-Fluorophenyl)-4-phenyl-2-((2-(trifluoromethyl)benzyl)thio)-1H-imidazole (DKL041-052, NCGC00601949): Follow General Method 1 using 3-fluoroaniline in the first step, and 1-(bromomethyl)-2-(trifluoromethyl)benzene in the final step. ¹H NMR (400 MHz, DMSO-d₆) δ 8.06 (s, 1H), 7.87-7.80 (m, 2H), 7.67 (d, J=7.9 Hz, 1H), 7.54 (ddd, J=14.6, 8.0, 6.2 Hz, 3H), 7.46 (t, J=7.5 Hz, 1H), 7.39 (t, J=7.7 Hz, 2H), 7.34-7.19 (m, 4H), 4.49 (s, 2H); LC-MS retention time (Method 1): 3.781 min; (Method 2): 6.618 min; HRMS: m/z (M+H)⁺=(Calculated for C₂₃H₁₇F₄N₂S 429.1043) found, 429.1053.

1-(3-Fluorophenyl)-2-((4-isopropylbenzyl)thio)-4-phenyl-1H-imidazole (DKL041-061, NCGC00602021): Follow General Method 1 using 3-fluoroaniline in the first step, and 1-(bromomethyl)-4-isopropylbenzene in the final step. ¹H NMR (400 MHz, DMSO-d₆) δ 8.03 (s, 1H), 7.88-7.81 (m, 2H), 7.52 (td, J=8.0, 6.4 Hz, 1H), 7.40 (t, J=7.7 Hz, 2H), 7.35-7.08 (m, 8H), 4.30 (s, 2H), 2.81 (hept, J=6.9 Hz, 1H), 1.14 (d, J=6.9 Hz, 6H); LC-MS retention time (Method 1): 3.818 min; (Method 2): 6.671 min; HRMS: m/z (M+H)⁺=(Calculated for C₂₅H₂₄FN₂S 403.1639) found, 403.1647.

4-(((1-(3-Fluorophenyl)-4-phenyl-1H-imidazol-2-yl)thio)methyl)benzenesulfonamide (DKL041-062, NCGC00602017): Follow General Method 1 using 3-fluoroaniline in the first step, and 4-(bromomethyl)benzene sulfonamide in the final step. ¹H NMR (400 MHz, DMSO-d₆) δ 8.03 (s, 1H), 7.87-7.79 (m, 2H), 7.71-7.64 (m, 2H), 7.55 (td, J=8.1, 6.4 Hz, 1H), 7.53-7.46 (m, 2H), 7.44-7.32 (m, 3H), 7.36-7.28 (m, 1H), 7.31-7.21 (m, 3H), 7.20 (dd, J=7.8, 2.0 Hz, 1H), 4.42 (s, 2H); LC-MS retention time (Method 1): 3.291 min; (Method 2): 5.049 min; HRMS: m/z (M+H)⁺=(Calculated for C₂₂H₁₉FN₃O₂S₂ 440.0897) found, 440.0900.

N-(4-(((1-(3-fluorophenyl)-4-phenyl-1H-imidazol-2-yl)thio)methyl)phenyl)acetamide (DKL041-063, NCGC00602026): Follow General Method 1 using 3-fluoroaniline in the first step, and N-(4-(bromomethyl)phenyl)acetamide in the final step. ¹H NMR (400 MHz, DMSO-d₆) δ 9.89 (s, 1H), 8.03 (s, 1H), 7.87-7.80 (m, 2H), 7.54 (td, J=8.1, 6.4 Hz, 1H), 7.45-7.36 (m, 4H), 7.26 (dddd, J=20.0, 13.7, 7.2, 2.2 Hz, 4H), 7.20-7.16 (m, 2H), 4.29 (s, 2H), 1.99 (s, 3H); LC-MS retention time (Method 1): 3.269 min; (Method 2): 5.007 min; HRMS: m/z (M+H)+=(Calculated for C₂₄H₂₁FN₃OS 418.1384) found, 418.1383.

2-((2-Fluoro-4-(trifluoromethyl)benzyl)thio)-1-(3-fluorophenyl)-4-phenyl-1H-imidazole (DKL041-058, NCGC00602024): Follow General Method 1 using 3-fluoroaniline in the first step, and 1-(bromomethyl)-2-fluoro-4-(trifluoromethyl)benzene in the final step. ¹H NMR (400 MHz, DMSO-d₆) δ 8.05 (s, 1H), 7.84-7.77 (m, 2H), 7.60-7.43 (m, 4H), 7.39 (t, J=7.7 Hz, 2H), 7.34-7.28 (m, 1H), 7.27-7.18 (m, 3H), 4.36 (s, 2H); LC-MS retention time (Method 1): 3.795 min; (Method 2): 6.692 min; HRMS: m/z (M+H)⁺=(Calculated for C₂₃H₁₆F₅N₂S 447.0949) found, 447.0958.

2-((4-(Tert-butyl)benzyl)thio)-1-(3-fluorophenyl)-4-phenyl-1H-imidazole (DKL041-060, NCGC00602019): Follow General Method 1 using 3-fluoroaniline in the first step, and 1-(bromomethyl)-4-(tert-butyl)benzene in the final step. ¹H NMR (400 MHz, DMSO-d₆) δ 8.03 (s, 1H), 7.91-7.78 (m, 2H), 7.58-7.47 (m, 1H), 7.40 (t, J=7.7 Hz, 2H), 7.35-7.20 (m, 4H), 7.20-7.14 (m, 4H), 4.31 (s, 2H), 1.22 (s, 9H); LC-MS retention time (Method 1): 3.868 min; (Method 2): 6.873 min; HRMS: m/z (M+H)⁺=(Calculated for C₂₆H₂₆FN₂S 417.1795) found, 417.1804.

2-((3-Bromobenzyl)thio)-1-(3-fluorophenyl)-4-phenyl-1H-imidazole (DKL041-059, NCGC00602020): Follow General Method 1 using 3-fluoroaniline in the first step, and 1-bromo-3-(bromomethyl)benzene in the final step. ¹H NMR (400 MHz, DMSO-d₆) δ 8.03 (s, 1H), 7.88-7.81 (m, 2H), 7.60-7.50 (m, 2H), 7.39 (dd, J=8.6, 6.6 Hz, 3H), 7.36-7.15 (m, 6H), 4.32 (s, 2H); LC-MS retention time (Method 1): 3.770 min; (Method 2): 6.550 min; HRMS: m/z (M+H)⁺=(Calculated for C₂₂H₁₇BrFN₂S 441.0255) found, 441.0264.

Tert-butyl(4-(((1-(3-fluorophenyl)-4-phenyl-1H-imidazol-2-yl)thio)methyl)phenyl) carbamate (DKL041-066, NCGC00602099): Follow General Method 1 using 3-fluoroaniline in the first step, and tert-butyl (4-(bromomethyl)phenyl)carbamate in the final step. ¹H NMR (400 MHz, DMSO-d₆) δ 9.29 (s, 1H), 8.03 (s, 1H), 7.87-7.81 (m, 2H), 7.54 (td, J=8.2, 6.4 Hz, 1H), 7.39 (t, J=7.7 Hz, 2H), 7.35-7.19 (m, 6H), 7.18-7.08 (m, 2H), 4.27 (s, 2H), 1.43 (s, 9H); LC-MS retention time (Method 1): 3.631 min; (Method 2): 6.156 min; HRMS: m/z (M+H)+=(Calculated for C₂₇H₂₇FN₃O₂S 476.1803) found, 476.1796.

4-(((1-(3-Fluorophenyl)-4-phenyl-1H-imidazol-2-yl)thio)methyl)benzamide (DKL041-069, NCGC00602096): Follow General Method 1 using 3-fluoroaniline in the first step, and 4-(bromomethyl)benzamide in the final step. ¹H NMR (400 MHz, DMSO-d₆) δ 8.03 (s, 1H), 7.93-7.78 (m, 3H), 7.78-7.69 (m, 2H), 7.54 (td, J=8.2, 6.4 Hz, 1H), 7.46-7.13 (m, 9H), 4.39 (s, 2H); LC-MS retention time (Method 1): 3.177 min; (Method 2): 4.885 min; HRMS: m/z (M+H)⁺=(Calculated for C₂₃H₁₉FN₃OS 404.1227) found, 404.1220.

1-(Bromomethyl)-4-(difluoromethyl)benzene (DKL041-070, NCGC00602098): Follow General Method 1 using 3-fluoroaniline in the first step, and 1-(bromomethyl)-4-(difluoromethyl)benzene in the final step. ¹H NMR (400 MHz, DMSO-d₆) δ 8.03 (s, 1H), 7.89-7.78 (m, 2H), 7.53 (td, J=8.2, 6.4 Hz, 1H), 7.46-7.37 (m, 6H), 7.34-7.15 (m, 4H), 6.96 (s, 1H), 4.39 (s, 2H); LC-MS retention time (Method 1): 3.643 min; (Method 2): 6.154 min; HRMS: m/z (M+H)⁺=(Calculated for C₂₃H₁₈F₃N₂S 411.1137) found, 411.1129.

1-(3-Fluorophenyl)-4-phenyl-2-((1-(4-(trifluoromethyl)phenyl)ethyl)thio)-1H-imidazole (DKL041-067, NCGC00602164): General Method 1 using 3-fluoroaniline in the first step, and 1-(1-bromoethyl)-4-(trifluoromethyl)benzene in the final step. ¹H NMR (400 MHz, DMSO-d₆) δ 8.01 (s, 1H), 7.89-7.77 (m, 2H), 7.56 (d, J=8.1 Hz, 2H), 7.49 (q, J=7.7 Hz, 1H), 7.39 (t, J=7.5 Hz, 4H), 7.33-7.23 (m, 2H), 7.11 (dd, J=8.7, 1.8 Hz, 2H), 4.76 (q, J=7.1 Hz, 1H), 1.64 (d, J=7.1 Hz, 3H); LC-MS retention time (Method 1): 3.805 min; (Method 2): 6.664 min; HRMS: m/z (M+H)⁺=(Calculated for C₂₄H₁₉F₄N₂S 443.1197) found, 443.1192.

2-((4-(Difluoromethoxy)benzyl)thio)-1-(3-fluorophenyl)-4-phenyl-1H-imidazole (DKL041-068, NCGC00602097): General Method 1 using 3-fluoroaniline in the first step, and 1-(bromomethyl)-4-(difluoromethoxy)benzene in the final step. ¹H NMR (400 MHz, DMSO-d₆) δ 8.03 (s, 1H), 7.86-7.81 (m, 2H), 7.54 (td, J=8.1, 6.4 Hz, 1H), 7.39 (t, J=7.7 Hz, 2H), 7.36-7.19 (m, 6H), 7.17 (s, 1H), 7.07-7.02 (m, 2H), 4.35 (s, 2H); LC-MS retention time (Method 1): 3.644 min; (Method 2): 6.157 min; HRMS: m/z (M+H)⁺=(Calculated for C₂₃H₁₈F₃N₂OS 427.1086) found, 427.1091.

4-(((1-(3-Fluorophenyl)-4-phenyl-1H-imidazol-2-yl)thio)methyl)benzonitrile (DKL041-085, NCGC00602282): General Method 1 using 3-fluoroaniline in the first step, and 4-(bromomethyl)benzonitrile in the final step. ¹H NMR (400 MHz, DMSO-d₆) δ 8.03 (s, 1H), 7.85-7.77 (m, 2H), 7.77-7.67 (m, 2H), 7.54 (td, J=8.2, 6.4 Hz, 1H), 7.50-7.44 (m, 2H), 7.39 (t, J=7.7 Hz, 2H), 7.36-7.26 (m, 1H), 7.30-7.19 (m, 3H), 4.41 (s, 2H); LC-MS retention time (Method 1): 3.581 min; (Method 2): 5.929 min; HRMS: m/z (M+H)⁺=(Calculated for C₂₃H₁₇FN₃S 386.1122) found, 386.1139.

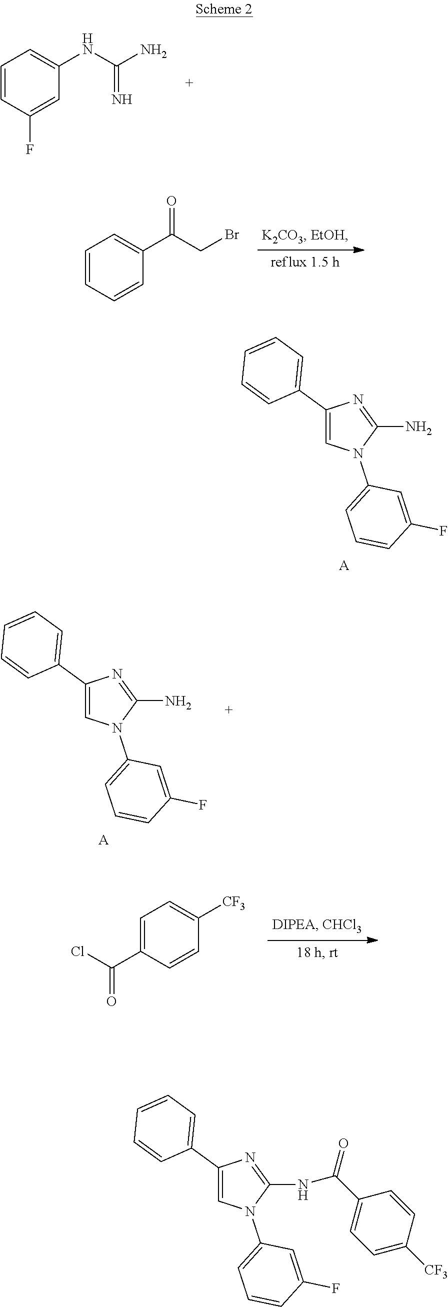

N-(1-(3-fluorophenyl)-4-phenyl-1H-imidazol-2-yl)-4-(trifluoromethyl)benzamide (DKL041-030, NCGC00601601): 1-(3-fluorophenyl)-4-phenyl-1H-imidazol-2-amine (50 mg, 0.20 mmol) and DIPEA (70 uL, 0.40 mmol) were combine in CHCl₃ (1.5 mL) before the dropwise addition of 4-(trifluoromethyl)benzoyl chloride (0.029 ml, 0.197 mmol). The reaction was allowed to stir 18 h, concentrated and purified on reverse phase as explained above. ¹H NMR (400 MHz, DMSO-d₆) δ 11.00 (s, 1H), 8.07 (d, J=16.4 Hz, 3H), 7.91-7.73 (m, 5H), 7.69-6.96 (m, 6H); LC-MS retention time (Method 1): 2.802 min; (Method 2): 6.472 min; HRMS: m/z (M+H)⁺=(Calculated for C₂₃H₁₆F₄N₃O 426.1224) found, 426.1233.

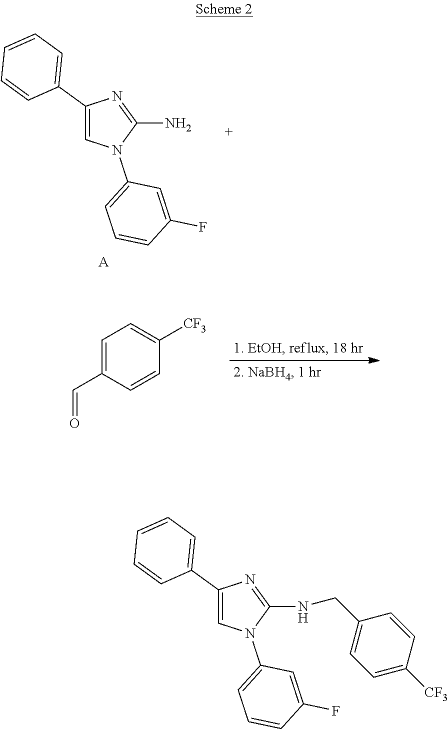

1-(3-Fluorophenyl)-4-phenyl-N-(4-(trifluoromethyl)benzyl)-1H-imidazol-2-amine (DKL041-034, NCGC00601632): Heated A (0.20 mmol), and 4-(trifluoromethyl)benzaldehyde (0.30 mmol) to reflux in EtOH (1.5 mL) 18 hr. Cool to rt and added NaBH₄ (0.40 mmol) stirred for 1 hr, added MeOH and water when complete. Filtered out the white precipitate and concentrated to an orange oil which was extracted with EtOAc and water, dried over MgSO₄, filtered and concentrated. The target compound was purified as mentioned above. ¹H NMR (400 MHz, DMSO-d₆) δ 7.82-7.58 (m, 8H), 7.58-7.20 (m, 6H), 4.71 (s, 2H); LC-MS retention time (Method 1): 3.889 min; (Method 2): 5.222 min; HRMS: m/z (M+H)⁺=(Calculated for C₂₃H₁₈F₄N₃ 412.1431) found, 412.1451.

N-benzyl-1-(3-fluorophenyl)-4-phenyl-1H-imidazol-2-amine, TFA (DKL042-047, NCGC00655680): Follow Scheme 2 using benzaldhyde instead of 4-(trifluoromethyl)benzaldehyde: ¹H NMR (400 MHz, DMSO-d₆) δ 12.71 (s, 1H), 8.53 (s, 1H), 7.90-7.53 (m, 5H), 7.52-7.07 (m, 9H), 4.82-4.33 (m, 2H); LC-MS retention time (Method 1): 3.189 min; (Method 2): 4.752 min; HRMS: m/z (M+H)⁺=(Calculated for C₂₂H₁₉FN₃ 344.1558) found, 344.1564.

1-(3-fluorophenyl)-4-phenyl-N-(3-(trifluoromethyl)benzyl)-1H-imidazol-2-amine, TFA (DKL042-046, NCGC00655683): Follow Scheme 2 using benzaldhyde instead of 4-(trifluoromethyl)benzaldehyde: ¹H NMR (400 MHz, DMSO-d₆) δ 12.74 (s, 1H), 8.51 (s, 1H), 7.80 (s, 1H), 7.75-7.53 (m, 5H), 7.53-7.18 (m, 6H), 4.69 (s, 2H); LC-MS retention time (Method 1): 3.297 min; (Method 2): 5.174 min; HRMS: m/z (M+H)⁺=(Calculated for C₂₃H₁₈F₄N₃ 412.1431) found, 412.1426.

N-(2-fluoro-3-(trifluoromethyl)benzyl)-1-(3-fluorophenyl)-4-phenyl-1H-imidazol-2-amine, TFA (DKL042-053, NCGC00655676): Scheme 2 using of 2-fluoro-3-(trifluoromethyl)benzaldehyde instead 4-(trifluoromethyl)benzaldehyde. LC-MS retention time (Method 1): 3.184 min; (Method 2): 5.203 min; HRMS: m/z (M+H)⁺=(Calculated for C₂₃H₁₇F₅N₃ 431.1368) found, 431.1369.

4-(((1-(3-fluorophenyl)-4-phenyl-1H-imidazol-2-yl)amino)methyl)benzonitrile, TFA (DKL042-050, NCGC00655674): Follow Scheme 2 using 4-formylbenzonitrile instead of 4-(trifluoromethyl)benzaldehyde: ¹H NMR (400 MHz, DMSO-d₆) δ 12.71 (s, 1H), 8.52 (s, 1H), 7.83 (d, J=8.0 Hz, 2H), 7.71-7.64 (m, 3H), 7.60 (d, J=8.2 Hz, 3H), 7.50 (d, J=7.9 Hz, 1H), 7.44 (s, 4H), 7.33 (s, 1H), 4.69 (s, 2H); LC-MS retention time (Method 1): 2.957 min; (Method 2): 4.594 min; HRMS: m/z (M+H)⁺=(Calculated for C₂₃H₁₈FN₄ 369.1510) found, 369.1464.

N-(3,4-difluorobenzyl)-1-(3-fluorophenyl)-4-phenyl-1H-imidazol-2-amine, TFA (DKL042-051, NCGC00655684): Follow Scheme 2 using 3,4-difluorobenzaldehyde. instead of 4-(trifluoromethyl)benzaldehyde: ¹H NMR (400 MHz, DMSO-d₆) δ 8.51 (s, 1H), 7.87-6.91 (m, 9H), 4.58 (s, 2H); LC-MS retention time (Method 1): 3.094 min; (Method 2): 4.952 min; HRMS: m/z (M+H)⁺=(Calculated for C₂₂H₁₇F₃N₃ 380.1369) found, 380.1367.

1-(3-fluorophenyl)-N-(3-methylbenzyl)-4-phenyl-1H-imidazol-2-amine, TFA (DKL042-048, NCGC00655675): Follow Scheme 2 using 3-methylbenzaldehyde instead of 4-(trifluoromethyl)benzaldehyde: ¹H NMR (400 MHz, DMSO-d₆) δ 12.69 (s, 1H), 7.79 (s, 1H), 7.74-7.63 (m, 3H), 7.61 (d, J=9.7 Hz, 1H), 7.52-7.42 (m, 4H), 7.36 (s, 1H), 7.24 (t, J=7.5 Hz, 1H), 7.21-7.14 (m, 2H), 7.09 (d, J=7.4 Hz, 1H), 4.62-4.56 (m, 2H), 2.29 (s, 3H); LC-MS retention time (Method 1): 3.262 min; (Method 2): 5.043 min; HRMS: m/z (M+H)⁺=(Calculated for C₂₃H₂₁FN₃ 358.1714) found, 358.1712.

N-(4-chlorobenzyl)-1-(3-fluorophenyl)-4-phenyl-1H-imidazol-2-amine, TFA (DKL042-052, NCGC00655679): Follow Scheme 2 using 4-chlorobenzaldehyde. instead of 4-(trifluoromethyl)benzaldehyde: ¹H NMR (400 MHz, DMSO-d₆) δ 12.70 (s, 1H), 7.77 (s, 1H), 7.72-7.64 (m, 3H), 7.60 (q, J=14.3, 13.6 Hz, 2H), 7.51-7.38 (m, 4H), 7.42 (s, 4H), 7.35 (s, 2H), 4.60 (s, 2H); LC-MS retention time (Method 1): 3.263 min; (Method 2): 5.030 min; HRMS: m/z (M+H)⁺=(Calculated for C₂₂H₁₈ClFN₃ 378.1168) found, 378.1171.

1-(3-fluorophenyl)-N-(3-methoxybenzyl)-4-phenyl-1H-imidazol-2-amine, TFA (DKL042-049, NCGC00655682): Follow Scheme 2 using 3-methoxybenzaldehyde instead of 4-(trifluoromethyl)benzaldehyde: ¹H NMR (400 MHz, DMSO-d₆) δ 12.67 (s, 1H), 8.40 (d, J=77.0 Hz, 1H), 7.88-7.51 (m, 5H), 7.49-7.15 (m, 7H), 7.02-6.79 (m, 2H), 4.54 (s, 2H), 3.72 (s, 3H); LC-MS retention time (Method 1): 3.180 min; (Method 2): 4.825 min; HRMS: m/z (M+H)⁺=(Calculated for C₂₃H₂₁FN₃O 374.1663) found, 374.1666.

4-(4-bromophenyl)-1-(3-fluorophenyl)-2-((4-(3-(trifluoromethyl)-3H-diazirin-3-yl)benzyl)thio)-1H-imidazole, TFA (DKL042-045, NCGC00655688): Follow General Method 1 using 3-fluoroaniline in the first step, and 3-(4-(bromomethyl)phenyl)-3-(trifluoromethyl)-3H-diazirine in the final step. ¹H NMR (400 MHz, DMSO-d₆) δ 8.09 (s, 1H), 7.80-7.74 (m, 2H), 7.62-7.54 (m, 2H), 7.52 (td, J=8.3, 6.4 Hz, 1H), 7.45-7.36 (m, 2H), 7.31 (td, J=8.6, 2.5 Hz, 1H), 7.27-7.12 (m, 4H), 4.36 (s, 2H); LC-MS retention time (Method 1): 3.880 min; (Method 2): 7.356 min; HRMS: m/z (M+H)⁺=(Calculated for C₂₄H₁₆BrF₄N₄S 549.0191) found, 549.0190.

1-(3-fluorophenyl)-N-(3-methoxy-4-(trifluoromethyl)benzyl)-4-phenyl-1H-imidazol-2-amine, TFA (DKL042-068, NCGC00658421): Follow Scheme 2 using 3-methoxy-4-(trifluoromethyl)benzaldehyde instead of 4-(trifluoromethyl)benzaldehyde: ¹H NMR (400 MHz, DMSO-d₆) δ 12.72 (s, 1H), 8.56 (s, 1H), 7.79 (s, 1H), 7.73-7.63 (m, 2H), 7.58 (d, J=8.0 Hz, 1H), 7.50 (d, J=7.5 Hz, 1H), 7.44 (s, 4H), 7.31 (s, 1H), 7.12 (d, J=8.0 Hz, 1H), 4.67 (s, 2H), 3.89 (s, 3H); LC-MS retention time (Method 1): 3.651 min; (Method 2): 5.279 min; HRMS: m/z (M+H)⁺=(Calculated for C₂₄H₂₀F₄N₃O 442.1537) found, 442.1533.

1-(3-fluorophenyl)-N-(3-methyl-4-(trifluoromethyl)benzyl)-4-phenyl-1H-imidazol-2-amine (DKL042-071, NCGC00666252): Follow Scheme 2 using 3-methyl-4-(trifluoromethyl)benzaldehyde instead of 4-(trifluoromethyl)benzaldehyde: ¹H NMR (400 MHz, DMSO-d₆) δ 12.69 (s, 1H), 7.94-7.52 (m, 4H), 7.50-6.98 (m, 9H), 4.65 (s, 2H), 2.44 (d, J=2.0 Hz, 3H); LC-MS retention time (Method 1): 3.380 min; (Method 2): 5.428 min; HRMS: m/z (M+H)⁺=(Calculated for C₂₄H₂₀F₄N₃ 426.1588) found, 426.1591.

N-(3-bromo-4-(trifluoromethyl)benzyl)-1-(3-fluorophenyl)-4-phenyl-1H-imidazol-2-amine, TFA (DKL042-070, NCGC00658419): Follow Scheme 2 using 3-bromo-4-(trifluoromethyl)benzaldehyde instead of 4-(trifluoromethyl)benzaldehyde: ¹H NMR (400 MHz, DMSO-d₆) δ 7.95 (s, 1H), 7.82 (d, J=8.1 Hz, 1H), 7.72-7.65 (m, 3H), 7.66 (s, 1H), 7.62 (d, J=8.1 Hz, 1H), 7.48 (s, 2H), 7.43 (s, 3H), 7.33 (s, 1H), 4.67 (s, 2H); LC-MS retention time (Method 1): 3.367 min; (Method 2): 5.427 min; HRMS: m/z (M+H)⁺=(Calculated for C₂₃H₁₈F₄N₃ 412.1431) found, 412.1451.

N-(3-chloro-4-(trifluoromethyl)benzyl)-1-(3-fluorophenyl)-4-phenyl-1H-imidazol-2-amine, TFA (DKL042-069, NCGC00658422): Follow Scheme 2 using 3-chloro-4-(trifluoromethyl)benzaldehyde instead of 4-(trifluoromethyl)benzaldehyde: ¹H NMR (400 MHz, DMSO-d₆) δ 7.95-7.74 (m, 2H), 7.74-7.54 (m, 4H), 7.46 (d, J=25.7 Hz, 4H), 4.67 (s, 2H); LC-MS retention time (Method 1): 3.731 min; (Method 2): 5.399 min; HRMS: m/z (M+H)⁺=(Calculated for C₂₃H₁₇ClF₄N₃ 446.1042) found, 446.1066.

The potency of several 15LOX/PEBP1 inhibitors with anti-ferroptotic effect was determined and are shown in Table 1 below.

TABLE 1
Potency of 15LOX/PEBP1 inhibitors.

| Cmpnd | | | | |
|---|---|---|---|---|
| no. | Registration No. | Structure | Name | Potency* |
| 1 | NCGC00599836- 01 | 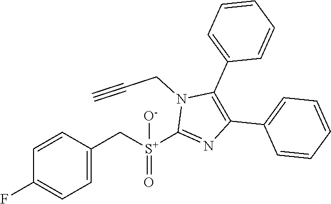 [SMILES: C#CCn1c([S+](=O)([O-])Cc2ccc(F)cc2)nc(-c2ccccc2)c1-c1ccccc1] | 2-((4-fluorobenzyl)sulfonyl)-4,5- diphenyl-1-(prop-2-yn-1-yl)-1H- imidazole | |
| 2 | NCGC00599837- 01 | 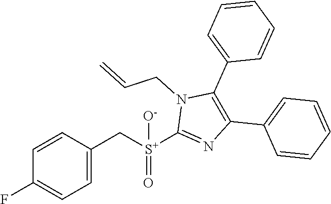 [SMILES: C=CCn1c([S+](=O)([O-])Cc2ccc(F)cc2)nc(-c2ccccc2)c1-c1ccccc1] | 1-allyl-2-((4- fluorobenzyl)sulfonyl)-4,5- diphenyl-1H-imidazole | |
| 3 | NCGC00599874- 01 | 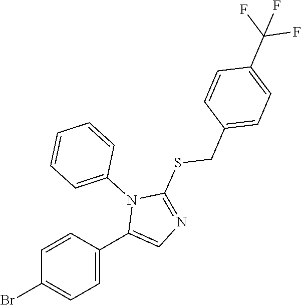 [SMILES: FC(F)(F)c1ccc(CSc2ncc(-c3ccc(Br)cc3)n2-c2ccccc2)cc1] | 5-(4-bromophenyl)-1-phenyl-2-((4- (trifluoromethyl)benzyl)thio)-1H- imidazole | |
| 4 | NCGC00599877- 01 | 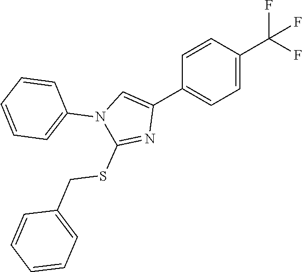 [SMILES: FC(F)(F)c1ccc(-c2cn(-c3ccccc3)c(SCc3ccccc3)n2)cc1] | 2-(benzylthio)-1-phenyl-4-(4- (trifluoromethyl)phenyl)-1H- imidazole | + |
| 5 | NCGC00599883- 01 | 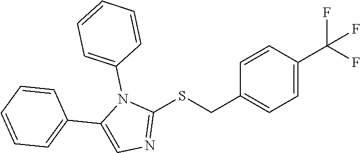 [SMILES: FC(F)(F)c1ccc(CSc2ncc(-c3ccccc3)n2-c2ccccc2)cc1] | 1,5-diphenyl-2-((4- (trifluoromethyl)benzyl)thio)-1H- imidazole | |
| 6 | NCGC00599898- 01 | 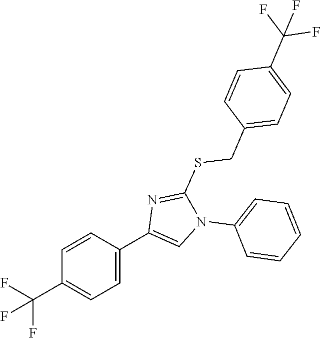 [SMILES: FC(F)(F)c1ccc(CSc2nc(-c3ccc(C(F)(F)F)cc3)cn2-c2ccccc2)cc1] | 1-phenyl-2-((4- (trifluoromethyl)benzyl)thio)-4-(4- (trifluoromethyl)phenyl)-1H- imidazole | + |
| 7 | NCGC00599904- 01 | 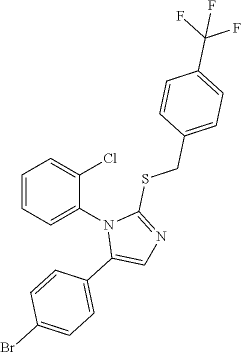 [SMILES: FC(F)(F)c1ccc(CSc2ncc(-c3ccc(Br)cc3)n2-c2ccccc2Cl)cc1] | 5-(4-bromophenyl)-1-(2- chlorophenyl)-2-((4- (trifluoromethyl)benzyl)thio)-1H- imidazole | |
| 8 | NCGC00599921- 01 | 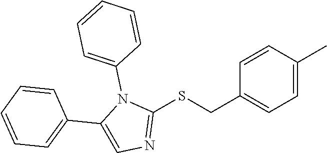 [SMILES: Cc1ccc(CSc2ncc(-c3ccccc3)n2-c2ccccc2)cc1] | 2-((4-methylbenzyl)thio)-1,5- diphenyl-1H-imidazole | |
| 9 | NCGC00599925- 01 | 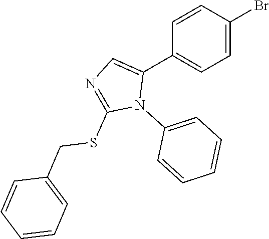 [SMILES: Brc1ccc(-c2cnc(SCc3ccccc3)n2-c2ccccc2)cc1] | 2-(benzylthio)-5-(4-bromophenyl)- 1-phenyl-1H-imidazole | |
| 10 | NCGC00599927- 01 | 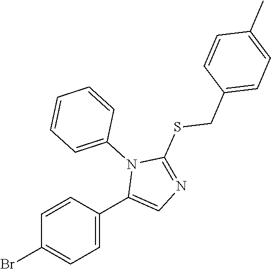 [SMILES: Cc1ccc(CSc2ncc(-c3ccc(Br)cc3)n2-c2ccccc2)cc1] | 5-(4-bromophenyl)-2-((4- methylbenzyl)thio)-1-phenyl-1H- imidazole | |
| 11 | NCGC00599971- 01 | 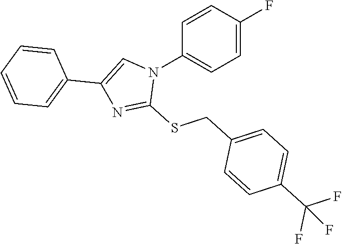 [SMILES: Fc1ccc(-n2cc(-c3ccccc3)nc2SCc2ccc(C(F)(F)F)cc2)cc1] | 1-(4-fluorophenyl)-4-phenyl-2-((4- (trifluoromethyl)benzyl)thio)-1H- imidazole | + |
| 12 | NCGC00599973- 08 | 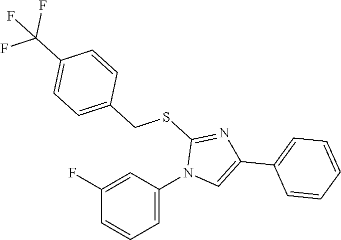 [SMILES: Fc1cccc(-n2cc(-c3ccccc3)nc2SCc2ccc(C(F)(F)F)cc2)c1] | 1-(3-fluorophenyl)-4-phenyl-2-((4- (trifluoromethyl)benzyl)thio)-1H- imidazole | ++ |
| 13 | NCGC00599991- 01 | 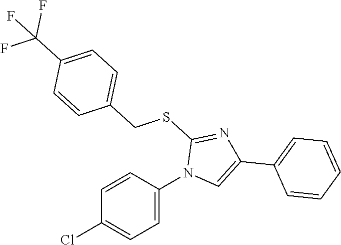 [SMILES: FC(F)(F)c1ccc(CSc2nc(-c3ccccc3)cn2-c2ccc(Cl)cc2)cc1] | 1-(4-chlorophenyl)-4-phenyl-2-((4- (trifluoromethyl)benzyl)thio)-1H- imidazole | + |
| 14 | NCGC00599992- 01 | 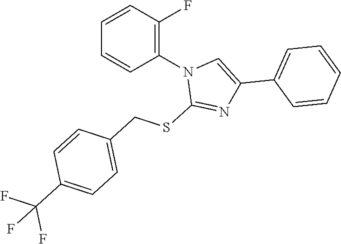 [SMILES: Fc1ccccc1-n1cc(-c2ccccc2)nc1SCc1ccc(C(F)(F)F)cc1] | 1-(2-fluorophenyl)-4-phenyl-2-((4- (trifluoromethyl)benzyl)thio)-1H- imidazole | + |
| 15 | NCGC00599993- 01 | 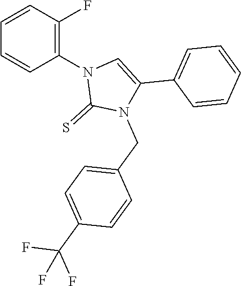 [SMILES: Fc1ccccc1-n1cc(-c2ccccc2)n(Cc2ccc(C(F)(F)F)cc2)c1=S] | 1-(2-fluorophenyl)-4-phenyl-3-(4- (trifluoromethyl)benzyl)-1,3- dihydro-2H-imidazole-2-thione | |
| 16 | NCGC00599997- 01 | 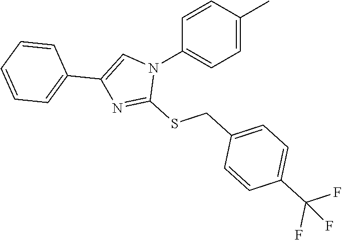 [SMILES: Cc1ccc(-n2cc(-c3ccccc3)nc2SCc2ccc(C(F)(F)F)cc2)cc1] | 4-phenyl-1-(p-tolyl)-2-((4- (trifluoromethyl)benzyl)thio)-1H- imidazole | + |
| 17 | NCGC00600001- 02 | 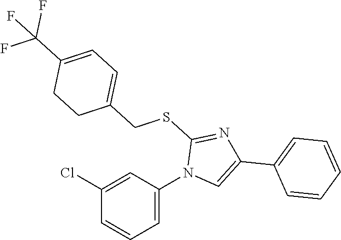 [SMILES: FC(F)(F)C1=CC=C(CSc2nc(-c3ccccc3)cn2-c2cccc(Cl)c2)CC1] | 1-(3-chlorophenyl)-4-phenyl-2-((4- (trifluoromethyl)benzyl)thio)-1H- imidazole | + |
| 18 | NCGC00600014- 01 | 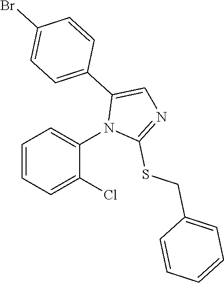 [SMILES: Clc1ccccc1-n1c(-c2ccc(Br)cc2)cnc1SCc1ccccc1] | 2-(benzylthio)-5-(4-bromophenyl)- 1-(2-chlorophenyl)-1H-imidazole | |
| 19 | NCGC00600017- 01 | 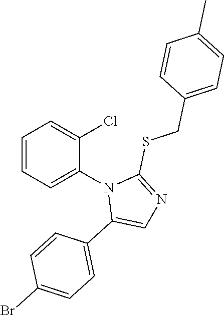 [SMILES: Cc1ccc(CSc2ncc(-c3ccc(Br)cc3)n2-c2ccccc2Cl)cc1] | 5-(4-bromophenyl)-1-(2- chlorophenyl)-2-((4- methylbenzyl)thio)-1H-imidazole | |
| 20 | NCGC00600066- 01 | 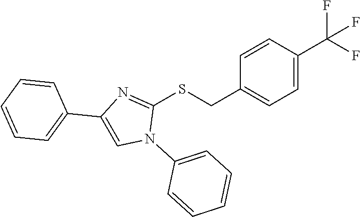 [SMILES: FC(F)(F)c1ccc(CSc2nc(-c3ccccc3)cn2-c2ccccc2)cc1] | 1,4-diphenyl-2-((4- (trifluoromethyl)benzyl)thio)-1H- imidazole | + |
| 21 | NCGC00600614- 01 | 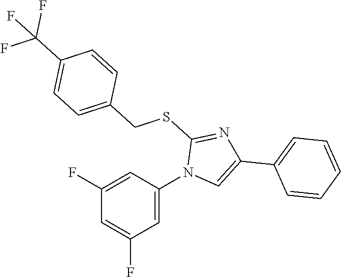 [SMILES: Fc1cc(F)cc(-n2cc(-c3ccccc3)nc2SCc2ccc(C(F)(F)F)cc2)c1] | 1-(3,5-difluorophenyl)-4-phenyl-2- ((4-(trifluoromethyl)benzyl)thio)- 1H-imidazole | dead |
| 22 | NCGC00600630- 01 | 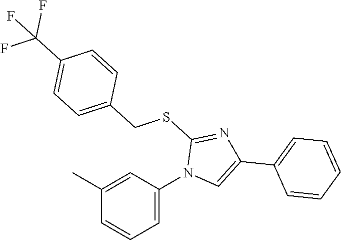 [SMILES: Cc1cccc(-n2cc(-c3ccccc3)nc2SCc2ccc(C(F)(F)F)cc2)c1] | 4-phenyl-1-(m-tolyl)-2-((4- (trifluoromethyl)benzyl)thio)-1H- imidazole | + |
| 23 | NCGC00600635- 01 | 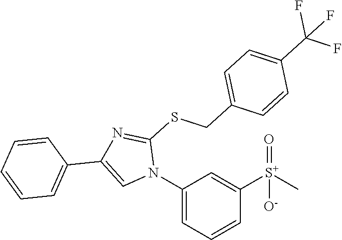 [SMILES: C[S+](=O)([O-])c1cccc(-n2cc(-c3ccccc3)nc2SCc2ccc(C(F)(F)F)cc2)c1] | 1-(3-(methylsulfonyl)phenyl)-4- phenyl-2-((4- (trifluoromethyl)benzyl)thio)-1H- imidazole | + |
| 24 | NCGC00600636- 01 | 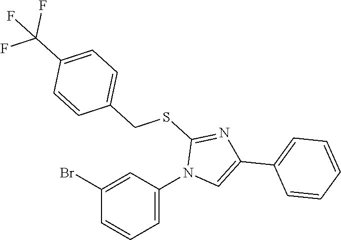 [SMILES: FC(F)(F)c1ccc(CSc2nc(-c3ccccc3)cn2-c2cccc(Br)c2)cc1] | 1-(3-bromophenyl)-4-phenyl-2-((4- (trifluoromethyl)benzyl)thio)-1H- imidazole | + |
| 25 | NCGC00600653- 01 | 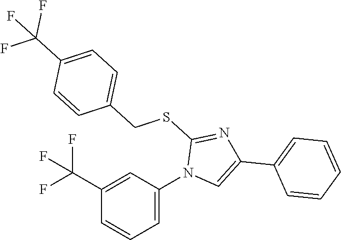 [SMILES: FC(F)(F)c1ccc(CSc2nc(-c3ccccc3)cn2-c2cccc(C(F)(F)F)c2)cc1] | 4-phenyl-2-((4- (trifluoromethyl)benzyl)thio)-1-(3- (trifluoromethyl)phenyl)-1H- imidazole | + |
| 26 | NCGC00600835- 01 | 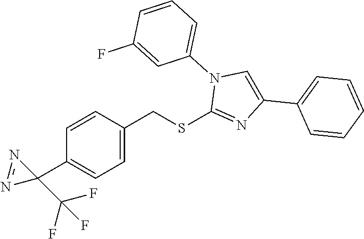 [SMILES: Fc1cccc(-n2cc(-c3ccccc3)nc2SCc2ccc(C3(C(F)(F)F)N=N3)cc2)c1] | 1-(3-fluorophenyl)-4-phenyl-2-((4- (3-(trifluoromethyl)-3H-diazirin-3- yl)benzyl)thio)-1H-imidazole | + |
| 27 | NCGC00600840- 01 | 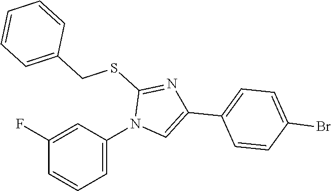 [SMILES: Fc1cccc(-n2cc(-c3ccc(Br)cc3)nc2SCc2ccccc2)c1] | 2-(benzylthio)-4-(4-bromophenyl)- 1-(3-fluorophenyl)-1H-imidazole | + |
| 28 | NCGC00600959- 01 | 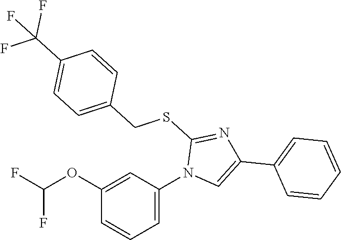 [SMILES: FC(F)Oc1cccc(-n2cc(-c3ccccc3)nc2SCc2ccc(C(F)(F)F)cc2)c1] | 1-(3-(difluoromethoxy)phenyl)-4- phenyl-2-((4- (trifluoromethyl)benzyl)thio)-1H- imidazole | + |
| 29 | NCGC00600960- 01 | 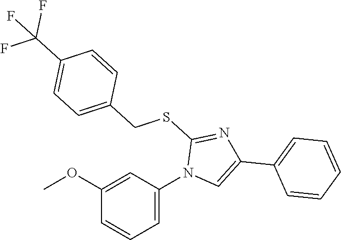 [SMILES: COc1cccc(-n2cc(-c3ccccc3)nc2SCc2ccc(C(F)(F)F)cc2)c1] | 1-(3-methoxyphenyl)-4-phenyl-2- ((4-(trifluoromethyl)benzyl)thio)- 1H-imidazole | + |
| 30 | NCGC00600973- 01 | 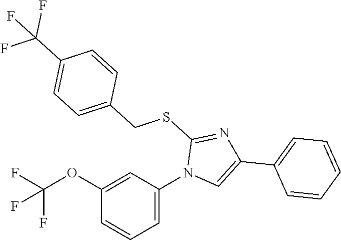 [SMILES: FC(F)(F)Oc1cccc(-n2cc(-c3ccccc3)nc2SCc2ccc(C(F)(F)F)cc2)c1] | 4-phenyl-1-(3- (trifluoromethoxy)phenyl)-2-((4- (trifluoromethyl)benzyl)thio)-1H- imidazole | + |
| 31 | NCGC00601601- 01 | 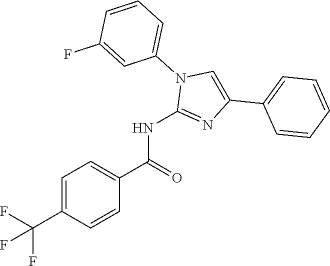 [SMILES: O=C(Nc1nc(-c2ccccc2)cn1-c1cccc(F)c1)c1ccc(C(F)(F)F)cc1] | N-(1-(3-fluorophenyl)-4-phenyl- 1H-imidazol-2-yl)-4- (trifluoromethyl)benzamide | + |
| 32 | NCGC00601632- 01 | 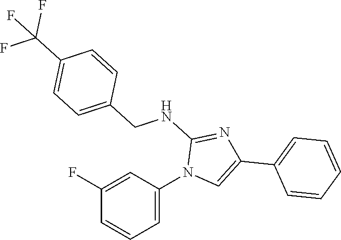 [SMILES: Fc1cccc(-n2cc(-c3ccccc3)nc2NCc2ccc(C(F)(F)F)cc2)c1] | 1-(3-fluorophenyl)-4-phenyl-N-(4- (trifluoromethyl)benzyl)-1H- imidazol-2-amine | ++ |
| 33 | NCGC00601881- 01 | 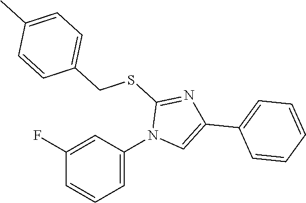 [SMILES: Cc1ccc(CSc2nc(-c3ccccc3)cn2-c2cccc(F)c2)cc1] | 1-(3-fluorophenyl)-2-((4- methylbenzyl)thio)-4-phenyl-1H- imidazole | + |
| 34 | NCGC00601930- 01 | 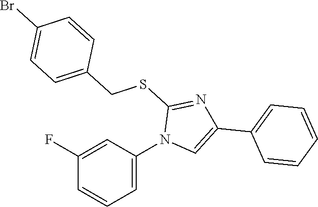 [SMILES: Fc1cccc(-n2cc(-c3ccccc3)nc2SCc2ccc(Br)cc2)c1] | 2-((4-bromobenzyl)thio)-1-(3- fluorophenyl)-4-phenyl-1H- imidazole | + |
| 35 | NCGC00601931- 01 | 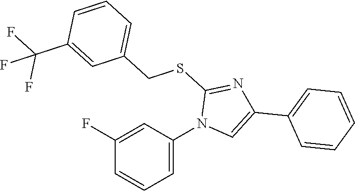 [SMILES: Fc1cccc(-n2cc(-c3ccccc3)nc2SCc2cccc(C(F)(F)F)c2)c1] | 1-(3-fluorophenyl)-4-phenyl-2-((3- (trifluoromethyl)benzyl)thio)-1H- imidazole | + |
| 36 | NCGC00601942- 01 | 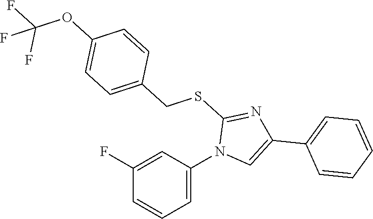 [SMILES: Fc1cccc(-n2cc(-c3ccccc3)nc2SCc2ccc(OC(F)(F)F)cc2)c1] | 1-(3-fluorophenyl)-4-phenyl-2-((4- (trifluoromethoxy)benzyl)thio)-1H- imidazole | + |
| 37 | NCGC00601943- 01 | 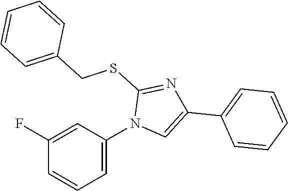 [SMILES: Fc1cccc(-n2cc(-c3ccccc3)nc2SCc2ccccc2)c1] | 2-(benzylthio)-1-(3-fluorophenyl)- 4-phenyl-1H-imidazole | + |
| 38 | NCGC00601944- 01 | 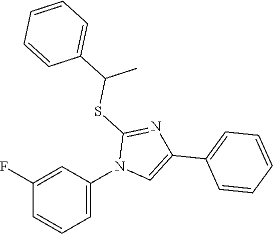 [SMILES: CC(Sc1nc(-c2ccccc2)cn1-c1cccc(F)c1)c1ccccc1] | 1-(3-fluorophenyl)-4-phenyl-2-((1- phenylethyl)thio)-1H-imidazole | + |
| 39 | NCGC00601947- 01 | 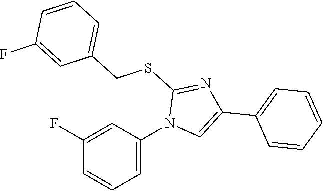 [SMILES: Fc1cccc(CSc2nc(-c3ccccc3)cn2-c2cccc(F)c2)c1] | 2-((3-chlorobenzyl)thio)-1-(3- fluorophenyl)-4-phenyl-1H- imidazole | + |
| 40 | NCGC00601948- 01 | 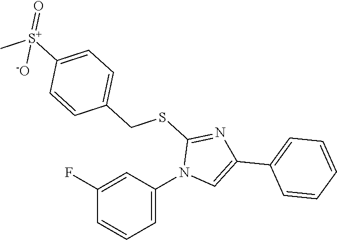 | 1-(3-fluorophenyl)-2-((4- (methylsulfonyl)benzyl)thio)-4- phenyl-1H-imidazole | + |
| 41 | NCGC00601949- 01 | 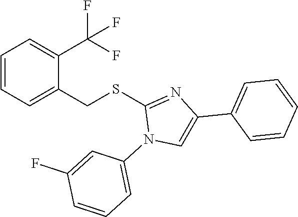 [SMILES: Fc1cccc(-n2cc(-c3ccccc3)nc2SCc2ccccc2C(F)(F)F)c1] | 1-(3-fluorophenyl)-4-phenyl-2-((2- (trifluoromethyl)benzyl)thio)-1H- imidazole | + |
| 42 | NCGC00601951- 01 | 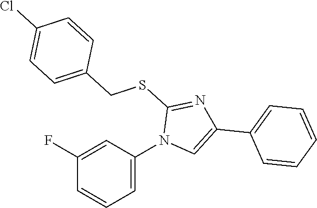 [SMILES: Fc1cccc(-n2cc(-c3ccccc3)nc2SCc2ccc(Cl)cc2)c1] | 2-((4-chlorobenzyl)thio)-1-(3- fluorophenyl)-4-phenyl-1H- imidazole | + |
| 43 | NCGC00601955- 01 | 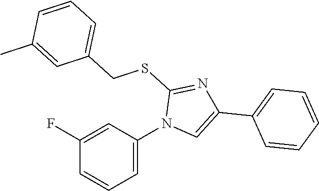 [SMILES: Cc1cccc(CSc2nc(-c3ccccc3)cn2-c2cccc(F)c2)c1] | 1-(3-fluorophenyl)-2-((3- methylbenzyl)thio)-4-phenyl-1H- imidazole | + |
| 44 | NCGC00602017- 01 | 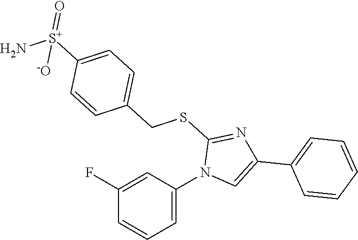 | 4-(((1-(3-fluorophenyl)-4-phenyl- 1H-imidazol-2- yl)thio)methyl)benzenesulfonamide | + |
| 45 | NCGC00602019- 01 | 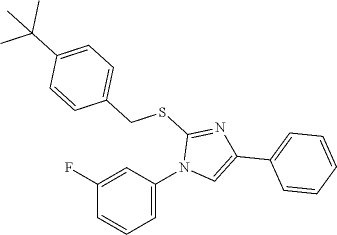 [SMILES: CC(C)(C)c1ccc(CSc2nc(-c3ccccc3)cn2-c2cccc(F)c2)cc1] | 2-((4-(tert-butyl)benzyl)thio)-1-(3- fluorophenyl)-4-phenyl-1H- imidazole | + |
| 46 | NCGC00602020- 01 | 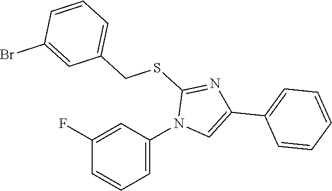 [SMILES: Fc1cccc(-n2cc(-c3ccccc3)nc2SCc2cccc(Br)c2)c1] | 2-((3-bromobenzyl)thio)-1-(3- fluorophenyl)-4-phenyl-1H- imidazole | + |
| 47 | NCGC00602021- 01 | 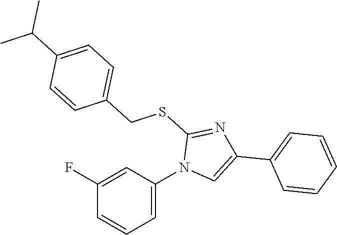 [SMILES: CC(C)c1ccc(CSc2nc(-c3ccccc3)cn2-c2cccc(F)c2)cc1] | 1-(3-fluorophenyl)-2-((4- isopropylbenzyl)thio)-4-phenyl- 1H-imidazole | + |
| 48 | NCGC00602024- 01 | 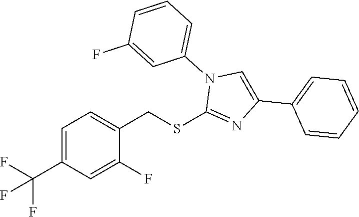 [SMILES: Fc1cccc(-n2cc(-c3ccccc3)nc2SCc2ccc(C(F)(F)F)cc2F)c1] | 2-((2-fluoro-4- (trifluoromethyl)benzyl)thio)-1-(3- fluorophenyl)-4-phenyl-1H- imidazole | + |
| 49 | NCGC00602026- 01 | 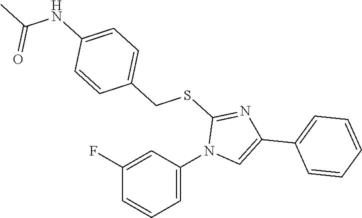 [SMILES: CC(=O)Nc1ccc(CSc2nc(-c3ccccc3)cn2-c2cccc(F)c2)cc1] | N-(4-(((1-(3-fluorophenyl)-4- phenyl-1H-imidazol-2- yl)thio)methyl)phenyl)acetamide | + |
| 50 | NCGC00602096- 01 | 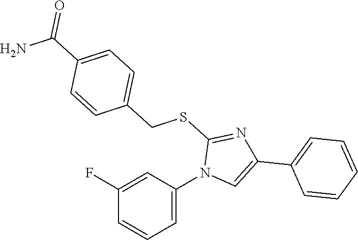 [SMILES: NC(=O)c1ccc(CSc2nc(-c3ccccc3)cn2-c2cccc(F)c2)cc1] | 4-(((1-(3-fluorophenyl)-4-phenyl- 1H-imidazol-2- yl)thio)methyl)benzamide | + |
| 51 | NCGC00602097- 01 | 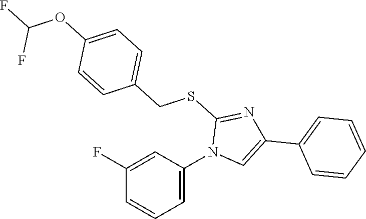 [SMILES: Fc1cccc(-n2cc(-c3ccccc3)nc2SCc2ccc(OC(F)F)cc2)c1] | 2-((4- (difluoromethoxy)benzyl)thio)-1- (3-fluorophenyl)-4-phenyl-1H- imidazole | + |
| 52 | NCGC00602098- 01 | 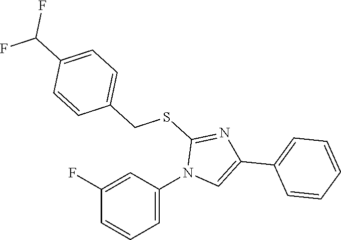 [SMILES: Fc1cccc(-n2cc(-c3ccccc3)nc2SCc2ccc(C(F)F)cc2)c1] | 2-((4-(difluoromethyl)benzyl)thio)- 1-(3-fluorophenyl)-4-phenyl-1H- imidazole | + |
| 53 | NCGC00602099- 01 | 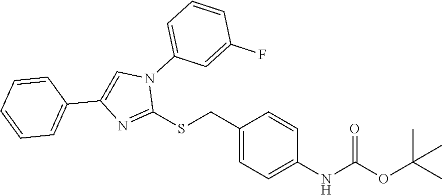 [SMILES: CC(C)(C)OC(=O)Nc1ccc(CSc2nc(-c3ccccc3)cn2-c2cccc(F)c2)cc1] | tert-butyl (4-(((1-(3-fluorophenyl)- 4-phenyl-1H-imidazol-2- yl)thio)methyl)phenyl)carbamate | + |
| 54 | NCGC00602164- 01 | 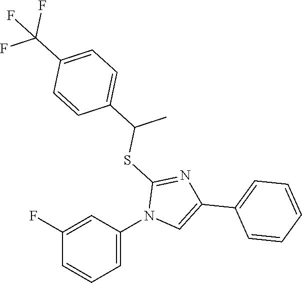 [SMILES: CC(Sc1nc(-c2ccccc2)cn1-c1cccc(F)c1)c1ccc(C(F)(F)F)cc1] | 1-(3-fluorophenyl)-4-phenyl-2-((1- (4- (trifluoromethyl)phenyl)ethyl)thio)- 1H-imidazole | + |
| 55 | NCGC00602282- 01 | 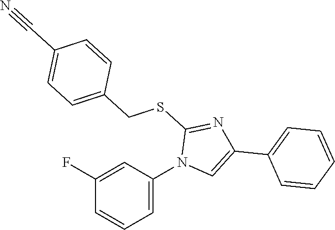 [SMILES: N#Cc1ccc(CSc2nc(-c3ccccc3)cn2-c2cccc(F)c2)cc1] | 4-(((1-(3-fluorophenyl)-4-phenyl- 1H-imidazol-2- yl)thio)methyl)benzonitrile | + |
| 56 | NCGC00655674- 01 | 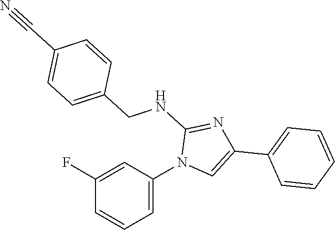 [SMILES: N#Cc1ccc(CNc2nc(-c3ccccc3)cn2-c2cccc(F)c2)cc1] | 4-(((1-(3-fluorophenyl)-4-phenyl- 1H-imidazol-2- yl)amino)methyl)benzonitrile | ++ |
| 57 | NCGC00655675- 01 | 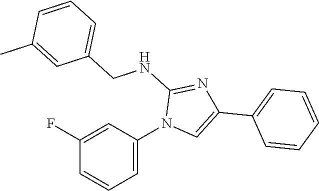 [SMILES: Cc1cccc(CNc2nc(-c3ccccc3)cn2-c2cccc(F)c2)c1] | 1-(3-fluorophenyl)-N-(3- methylbenzyl)-4-phenyl-1H- imidazol-2-amine | ++ |
| 58 | NCGC00655676- 01 | 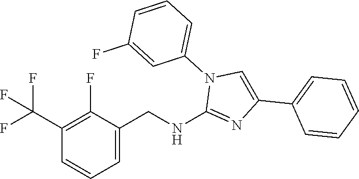 [SMILES: Fc1cccc(-n2cc(-c3ccccc3)nc2NCc2cccc(C(F)(F)F)c2F)c1] | N-(2-fluoro-3- (trifluoromethyl)benzyl)-1-(3- fluorophenyl)-4-phenyl-1H- imidazol-2-amine | ++ |
| 59 | NCGC00655679- 01 | 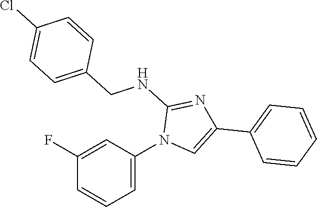 [SMILES: Fc1cccc(-n2cc(-c3ccccc3)nc2NCc2ccc(Cl)cc2)c1] | N-(4-chlorobenzyl)-1-(3- fluorophenyl)-4-phenyl-1H- imidazol-2-amine | ++ |
| 60 | NCGC00655679- 02 | 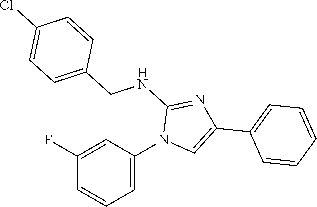 [SMILES: Fc1cccc(-n2cc(-c3ccccc3)nc2NCc2ccc(Cl)cc2)c1] | N-(4-chlorobenzyl)-1-(3- fluorophenyl)-4-phenyl-1H- imidazol-2-amine | |
| 61 | NCGC00655680- 01 | 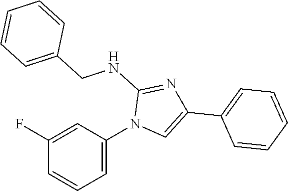 [SMILES: Fc1cccc(-n2cc(-c3ccccc3)nc2NCc2ccccc2)c1] | N-benzyl-1-(3-fluorophenyl)-4- phenyl-1H-imidazol-2-amine | ++ |
| 62 | NCGC00655682- 01 | 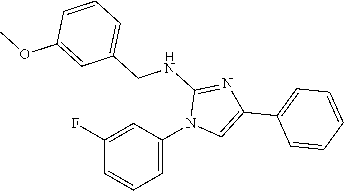 [SMILES: COc1cccc(CNc2nc(-c3ccccc3)cn2-c2cccc(F)c2)c1] | 1-(3-fluorophenyl)-N-(3- methoxybenzyl)-4-phenyl-1H- imidazol-2-amine | ++ |
| 63 | NCGC00655683- 01 | 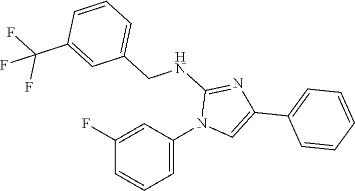 [SMILES: Fc1cccc(-n2cc(-c3ccccc3)nc2NCc2cccc(C(F)(F)F)c2)c1] | 1-(3-fluorophenyl)-4-phenyl-N-(3- (trifluoromethyl)benzyl)-1H- imidazol-2-amine | ++ |
| 64 | NCGC00655684- 01 | 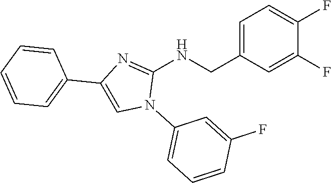 [SMILES: Fc1cccc(-n2cc(-c3ccccc3)nc2NCc2ccc(F)c(F)c2)c1] | N-(3,4-difluorobenzyl)-1-(3- fluorophenyl)-4-phenyl-1H- imidazol-2-amine | ++ |
| 65 | NCGC00655688- 01 | 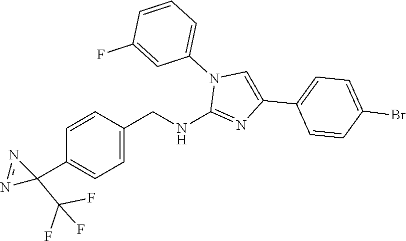 [SMILES: Fc1cccc(-n2cc(-c3ccc(Br)cc3)nc2NCc2ccc(C3(C(F)(F)F)N=N3)cc2)c1] | 4-(4-bromophenyl)-1-(3- fluorophenyl)-2-((4-(3- (trifluoromethyl)-3H-diazirin-3- yl)benzyl)thio)-1H-imidazole | + |
| 66 | NCGC00658419- 01 | 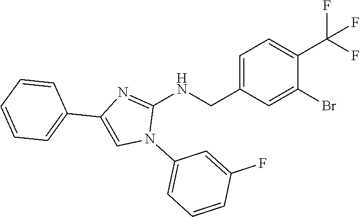 [SMILES: Fc1cccc(-n2cc(-c3ccccc3)nc2NCc2ccc(C(F)(F)F)c(Br)c2)c1] | N-(3-bromo-4- (trifluoromethyl)benzyl)-1-(3- fluorophenyl)-4-phenyl-1H- imidazol-2-amine | ++ |
| 67 | NCGC00658421- 02 | 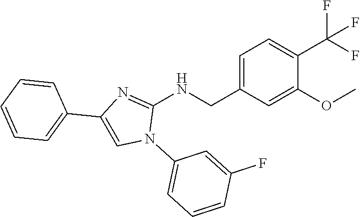 [SMILES: COc1cc(CNc2nc(-c3ccccc3)cn2-c2cccc(F)c2)ccc1C(F)(F)F] | 1-(3-fluorophenyl)-N-(3-methoxy- 4-(trifluoromethyl)benzyl)-4- phenyl-1H-imidazol-2-amine | ++ |
| 68 | NCGC00658422- 01 | 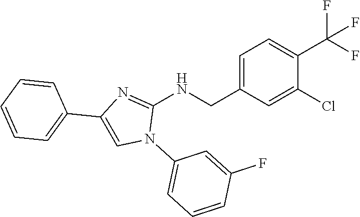 [SMILES: Fc1cccc(-n2cc(-c3ccccc3)nc2NCc2ccc(C(F)(F)F)c(Cl)c2)c1] | N-(3-chloro-4- (trifluoromethyl)benzyl)-1-(3- fluorophenyl)-4-phenyl-1H- imidazol-2-amine | ++ |
| 69 | NCGC00666252- 01 | 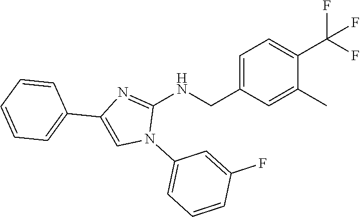 [SMILES: Cc1cc(CNc2nc(-c3ccccc3)cn2-c2cccc(F)c2)ccc1C(F)(F)F] | 1-(3-fluorophenyl)-N-(3-methyl-4- (trifluoromethyl)benzyl)-4-phenyl- 1H-imidazol-2-amine | ++ |
| 70 | NCGC00686635- 01 | 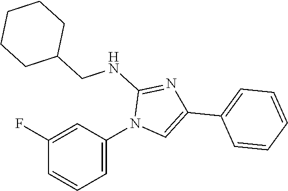 [SMILES: Fc1cccc(-n2cc(-c3ccccc3)nc2NCC2CCCCC2)c1] | 2-((cyclohexylmethyl)thio)-1-(3- fluorophenyl)-4-phenyl-1H- imidazole | |
| 71 | NCGC00687004- 01 | 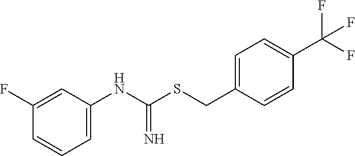 [SMILES: N=C(Nc1cccc(F)c1)SCc1ccc(C(F)(F)F)cc1] | 4-(trifluoromethyl)benzyl (3- fluorophenyl)carbamimidothioate | |
| 72 | NCGC00687170- 01 | 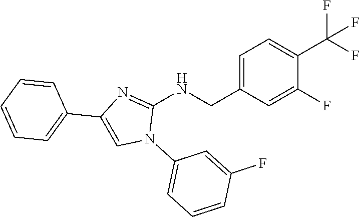 [SMILES: Fc1cccc(-n2cc(-c3ccccc3)nc2NCc2ccc(C(F)(F)F)c(F)c2)c1] | N-(3-fluoro-4- (trifluoromethyl)benzyl)-1-(3- fluorophenyl)-4-phenyl-1H- imidazol-2-amine | |
| 73 | NCGC00687402- 01 | 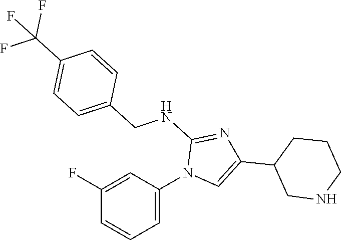 [SMILES: Fc1cccc(-n2cc(C3CCCNC3)nc2NCc2ccc(C(F)(F)F)cc2)c1] | 1-(3-fluorophenyl)-4-(piperidin-3- yl)-N-(4-(trifluoromethyl)benzyl)- 1H-imidazol-2-amine | |
| 74 | NCGC00687409- 01 | 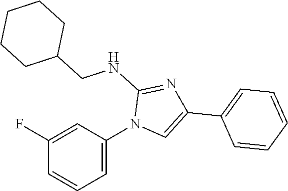 [SMILES: Fc1cccc(-n2cc(-c3ccccc3)nc2NCC2CCCCC2)c1] | N-(cyclohexylmethyl)-1-(3- fluorophenyl)-4-phenyl-1H- imidazol-2-amine | |
| 75 | NCGC00687413- 01 | 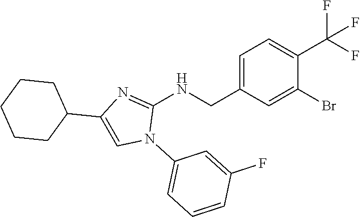 [SMILES: Fc1cccc(-n2cc(C3CCCCC3)nc2NCc2ccc(C(F)(F)F)c(Br)c2)c1] | N-(3-bromo-4- (trifluoromethyl)benzyl)-4- cyclohexyl-1-(3-fluorophenyl)-1H- imidazol-2-amine | |
| 76 | NCGC00687757- 01 | 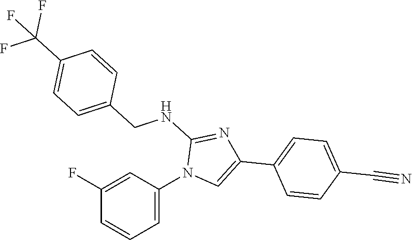 [SMILES: N#Cc1ccc(-c2cn(-c3cccc(F)c3)c(NCc3ccc(C(F)(F)F)cc3)n2)cc1] | 4-(1-(3-fluorophenyl)-2-((4- (trifluoromethyl)benzyl)amino)- 1H-imidazol-4-yl)benzonitrile | |
| 77 | NCGC00687758- 01 | 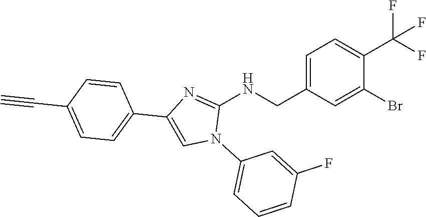 [SMILES: C#Cc1ccc(-c2cn(-c3cccc(F)c3)c(NCc3ccc(C(F)(F)F)c(Br)c3)n2)cc1] | N-(3-bromo-4- (trifluoromethyl)benzyl)-4-(4- ethynylphenyl)-1-(3-fluorophenyl)- 1H-imidazol-2-amine | |
| 78 | NCGC00687759- 01 | 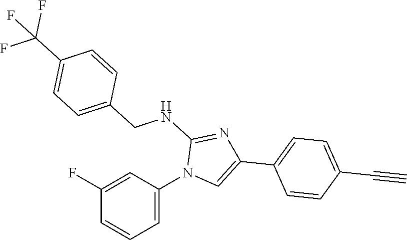 [SMILES: C#Cc1ccc(-c2cn(-c3cccc(F)c3)c(NCc3ccc(C(F)(F)F)cc3)n2)cc1] | 4-(4-ethynylphenyl)-1-(3- fluorophenyl)-N-(4- (trifluoromethyl)benzyl)-1H- imidazol-2-amine | |
| 79 | NCGC00687760- 01 | 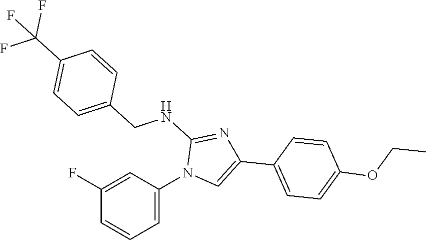 [SMILES: CCOc1ccc(-c2cn(-c3cccc(F)c3)c(NCc3ccc(C(F)(F)F)cc3)n2)cc1] | 4-(4-ethoxyphenyl)-1-(3- fluorophenyl)-N-(4- (trifluoromethyl)benzyl)-1H- imidazol-2-amine | |
| 80 | NCGC00687761- 01 | 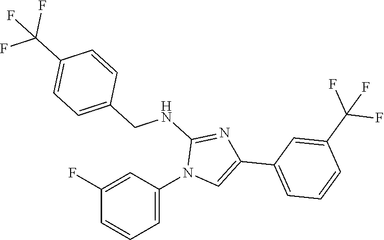 [SMILES: Fc1cccc(-n2cc(-c3cccc(C(F)(F)F)c3)nc2NCc2ccc(C(F)(F)F)cc2)c1] | 1-(3-fluorophenyl)-N-(4- (trifluoromethyl)benzyl)-4-(3- (trifluoromethyl)phenyl)-1H- imidazol-2-amine | |
| 81 | NCGC00687762- 01 | 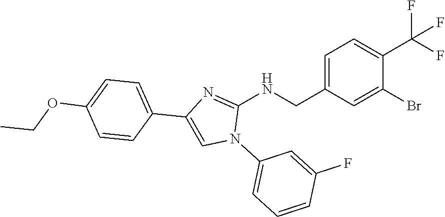 [SMILES: CCOc1ccc(-c2cn(-c3cccc(F)c3)c(NCc3ccc(C(F)(F)F)c(Br)c3)n2)cc1] | N-(3-bromo-4- (trifluoromethyl)benzyl)-4-(4- ethoxyphenyl)-1-(3-fluorophenyl)- 1H-imidazol-2-amine | |
| 82 | NCGC00687764- 01 | 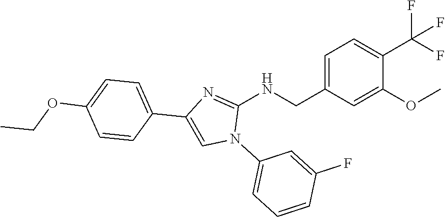 [SMILES: CCOc1ccc(-c2cn(-c3cccc(F)c3)c(NCc3ccc(C(F)(F)F)c(OC)c3)n2)cc1] | 4-(4-ethoxyphenyl)-1-(3- fluorophenyl)-N-(3-methoxy-4- (trifluoromethyl)benzyl)-1H- imidazol-2-amine | |
| 83 | NCGC00687765- 01 | 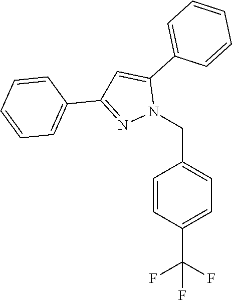 [SMILES: FC(F)(F)c1ccc(Cn2nc(-c3ccccc3)cc2-c2ccccc2)cc1] | 3,5-diphenyl-1-(4- (trifluoromethyl)benzyl)-1H- pyrazole | |
| 84 | NCGC00687771- 01 | 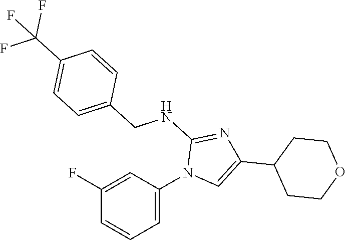 [SMILES: Fc1cccc(-n2cc(C3CCOCC3)nc2NCc2ccc(C(F)(F)F)cc2)c1] | 1-(3-fluorophenyl)-4-(tetrahydro- 2H-pyran-4-yl)-N-(4- (trifluoromethyl)benzyl)-1H- imidazol-2-amine | |
| 85 | NCGC00687772- 01 | 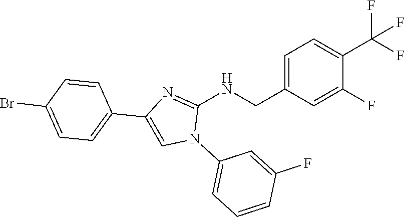 [SMILES: Fc1cccc(-n2cc(-c3ccc(Br)cc3)nc2NCc2ccc(C(F)(F)F)c(F)c2)c1] | 4-(4-bromophenyl)-N-(3-fluoro-4- (trifluoromethyl)benzyl)-1-(3- fluorophenyl)-1H-imidazol-2- amine | |
| 86 | NCGC00687776- 01 | 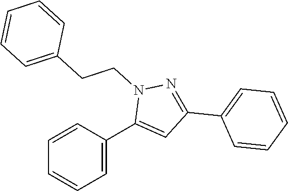 [SMILES: c1ccc(CCn2nc(-c3ccccc3)cc2-c2ccccc2)cc1] | 1-phenethyl-3,5-diphenyl-1H- pyrazole | |
| 87 | NCGC00687778- 01 | 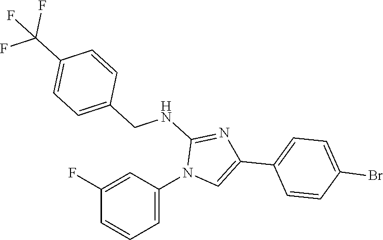 [SMILES: Fc1cccc(-n2cc(-c3ccc(Br)cc3)nc2NCc2ccc(C(F)(F)F)cc2)c1] | 4-(4-bromophenyl)-1-(3- fluorophenyl)-N-(4- (trifluoromethyl)benzyl)-1H- imidazol-2-amine | |
| 88 | NCGC00687786- 01 | 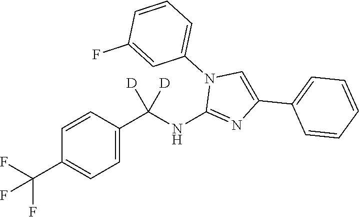 [SMILES: [2H]C([2H])(Nc1nc(-c2ccccc2)cn1-c1cccc(F)c1)c1ccc(C(F)(F)F)cc1] | 1-(3-fluorophenyl)-4-phenyl-N-((4- (trifluoromethyl)phenyl)methyl- d2)-1H-imidazol-2-amine | |
| 89 | NCGC00687788- 01 | 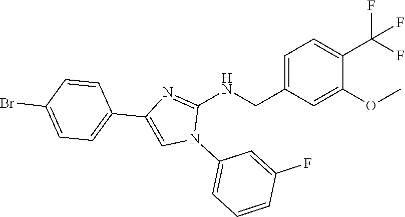 [SMILES: COc1cc(CNc2nc(-c3ccc(Br)cc3)cn2-c2cccc(F)c2)ccc1C(F)(F)F] | 4-(4-bromophenyl)-1-(3- fluorophenyl)-N-(3-methoxy-4- (trifluoromethyl)benzyl)-1H- imidazol-2-amine | |
| 90 | NCGC00687799- 01 | 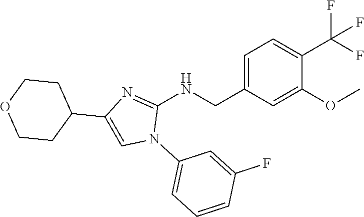 [SMILES: COc1cc(CNc2nc(C3CCOCC3)cn2-c2cccc(F)c2)ccc1C(F)(F)F] | 1-(3-fluorophenyl)-N-(3-methoxy- 4-(trifluoromethyl)benzyl)-4- (tetrahydro-2H-pyran-4-yl)-1H- imidazol-2-amine | |
| 91 | NCGC00687806- 01 | 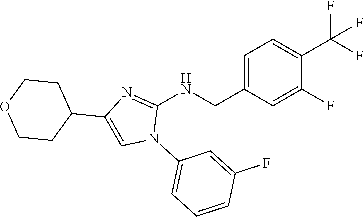 [SMILES: Fc1cccc(-n2cc(C3CCOCC3)nc2NCc2ccc(C(F)(F)F)c(F)c2)c1] | N-(3-fluoro-4- (trifluoromethyl)benzyl)-1-(3- fluorophenyl)-4-(tetrahydro-2H- pyran-4-yl)-1H-imidazol-2-amine | |
| 92 | NCGC00687808- 01 | 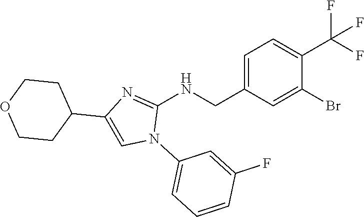 [SMILES: Fc1cccc(-n2cc(C3CCOCC3)nc2NCc2ccc(C(F)(F)F)c(Br)c2)c1] | N-(3-bromo-4- (trifluoromethyl)benzyl)-1-(3- fluorophenyl)-4-(tetrahydro-2H- pyran-4-yl)-1H-imidazol-2-amine | |
| 93 | NCGC00687813- 01 | 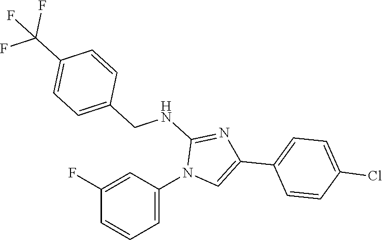 [SMILES: Fc1cccc(-n2cc(-c3ccc(Cl)cc3)nc2NCc2ccc(C(F)(F)F)cc2)c1] | 4-(4-chlorophenyl)-1-(3- fluorophenyl)-N-(4- (trifluoromethyl)benzyl)-1H- imidazol-2-amine | |
| 94 | NCGC00687852- 01 | 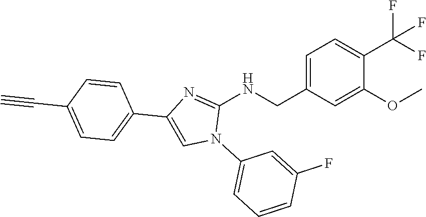 [SMILES: C#Cc1ccc(-c2cn(-c3cccc(F)c3)c(NCc3ccc(C(F)(F)F)c(OC)c3)n2)cc1] | 4-(4-ethynylphenyl)-1-(3- fluorophenyl)-N-(3-methoxy-4- (trifluoromethyl)benzyl)-1H- imidazol-2-amine | |
| 95 | NCGC00687861- 01 | 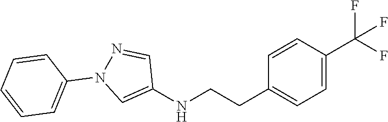 [SMILES: FC(F)(F)c1ccc(CCNc2cnn(-c3ccccc3)c2)cc1] | 1-phenyl-N-(4- (trifluoromethyl)phenethyl)-1H- pyrazol-4-amine | |
| 96 | NCGC00687954- 01 | 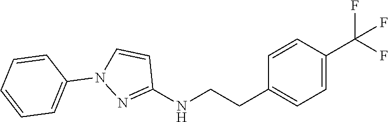 [SMILES: FC(F)(F)c1ccc(CCNc2ccn(-c3ccccc3)n2)cc1] | 1-phenyl-N-(4- (trifluoromethyl)phenethyl)-1H- pyrazol-3-amine | |
| 97 | NCGC00688012- 01 | 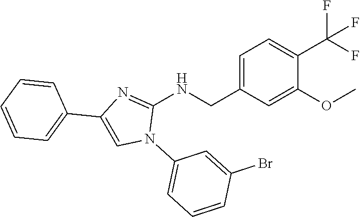 [SMILES: COc1cc(CNc2nc(-c3ccccc3)cn2-c2cccc(Br)c2)ccc1C(F)(F)F] | 1-(3-bromophenyl)-N-(3-methoxy- 4-(trifluoromethyl)benzyl)-4- phenyl-1H-imidazol-2-amine | |
| 98 | NCGC00688013- 01 | 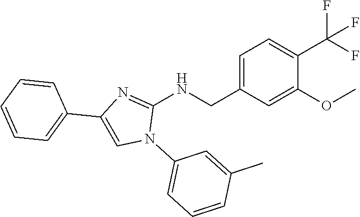 [SMILES: COc1cc(CNc2nc(-c3ccccc3)cn2-c2cccc(C)c2)ccc1C(F)(F)F] | N-(3-methoxy-4- (trifluoromethyl)benzyl)-4-phenyl- 1-(m-tolyl)-1H-imidazol-2-amine | |
| 99 | NCGC00688018- 01 | 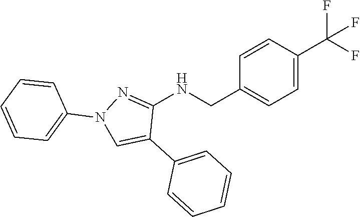 [SMILES: FC(F)(F)c1ccc(CNc2nn(-c3ccccc3)cc2-c2ccccc2)cc1] | 1,4-diphenyl-N-(4- (trifluoromethyl)benzyl)-1H- pyrazol-3-amine | |
| 100 | NCGC00689149- 01 | 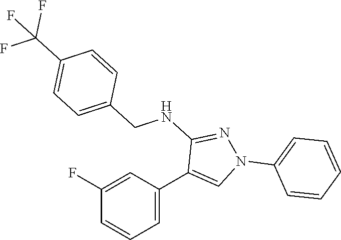 [SMILES: Fc1cccc(-c2cn(-c3ccccc3)nc2NCc2ccc(C(F)(F)F)cc2)c1] | 4-(3-fluorophenyl)-1-phenyl-N-(4- (trifluoromethyl)benzyl)-1H- pyrazol-3-amine | |
| 101 | NCGC00689325- 01 | 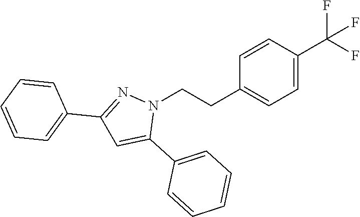 [SMILES: FC(F)(F)c1ccc(CCn2nc(-c3ccccc3)cc2-c2ccccc2)cc1] | 3,5-diphenyl-1-(4- (trifluoromethyl)phenethyl)-1H- pyrazole | |
| 102 | NCGC00689357- 01 | 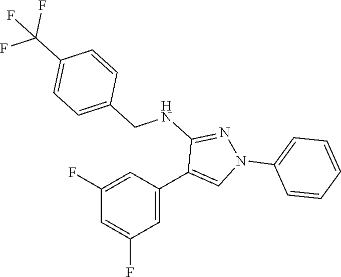 [SMILES: Fc1cc(F)cc(-c2cn(-c3ccccc3)nc2NCc2ccc(C(F)(F)F)cc2)c1] | 4-(3,5-difluorophenyl)-1-phenyl-N- (4-(trifluoromethyl)benzyl)-1H- pyrazol-3-amine | |
| 103 | NCGC00689481- 01 | 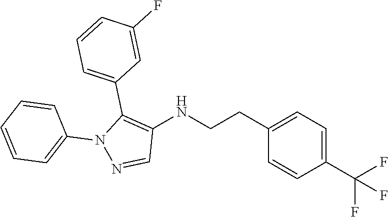 [SMILES: Fc1cccc(-c2c(NCCc3ccc(C(F)(F)F)cc3)cnn2-c2ccccc2)c1] | 5-(3-fluorophenyl)-1-phenyl-N-(4- (trifluoromethyl)phenethyl)-1H- pyrazol-4-amine | |
| 104 | NCGC00843079- 01 | 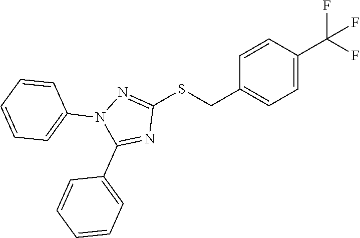 [SMILES: FC(F)(F)c1ccc(CSc2nc(-c3ccccc3)n(-c3ccccc3)n2)cc1] | 1,5-diphenyl-3-((4- (trifluoromethyl)benzyl)thio)-1H- 1,2,4-triazole | |
| 105 | NCGC00843083- 01 | 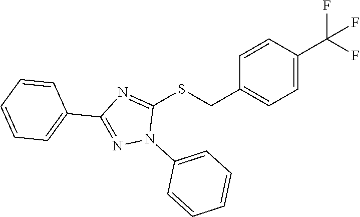 [SMILES: FC(F)(F)c1ccc(CSc2nc(-c3ccccc3)nn2-c2ccccc2)cc1] | 1,3-diphenyl-5-((4- (trifluoromethyl)benzyl)thio)-1H- 1,2,4-triazole | |

*Potency: +++ = 1-100 nM; ++ = 101-1000 nM; + > 1 μM

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

This listing of claims replaces all prior versions and listings of claims in the application:

1. A compound having a structure according to Formula I: 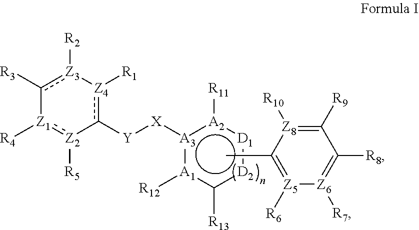
wherein
A₁, A₂, and A₃ are independently C, N, or S;
D₁ and D₂ are independently O, CR′, NR′ or S wherein R′ is absent or hydrogen, C₁-C₆ alkyl, C₂-C₆ alkenyl, C₂-C₆ alkynyl, aryl, alkylaryl, heteroaryl, or alkylheteroaryl, wherein R′ is optionally substituted or unsubstituted;
X is absent or O, S, SO, SO₂, CHR′, or NR′, wherein R′ is hydrogen, alkyl, or cycloalkyl;
Y is a bond, —C₁-C₃ alkyl-, —C₂-C₃ alkenyl-, —C₁-C₃ haloalkyl-, —C₂-C₃ haloalkenyl-, —C₁-C₃ alkyloxy-, —C₁-C₃ alkylamine-, —C₁-C₃ alkylamide-, —C₁-C₃ alkylsulfide-, —C₁-C₃ alkylthiol-, —C₁-C₃alkylsulfoxide-, —C₁-C₃ alkylsulfonyl-, —C₁-C₃ alkylsulfonamide-, —C₁-C₃ ester-, or cyclic, and wherein Y is optionally substituted with one or more groups;
Z₁, Z₂, Z₃, Z₄, Z₅, Z₆, Z₇, and Z₈ are independently C, CH, O, S, or N;
R₁, R₂, R₄, and R₅ are independently absent or hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁, R₂, R₄, and R₅ are optionally substituted with one or more groups;
R₃ is hydrogen, halogen, hydroxyl, amine, C₁-C₆ alkyl, C₂-C₆ alkenyl, C₂-C₆alkynyl, C₁-C₆ haloalkyl, C₂-C₆ haloalkenyl, C₂-C₆ haloalkynyl, C₁-C₆alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₆ to R₁₀ are optionally substituted with one or more groups;
R₁₁ is absent or hydrogen, cyano, C₁-C₆ alkyl, C₂-C₆ alkenyl, C₂-C₆ alkynyl, aryl, alkylaryl, heteroaryl, or alkylheteroaryl, or R₁₁ can combine with D₁ to form a six membered ring, wherein R₁₁ is optionally substituted or unsubstituted;
R₁₂ is absent or hydrogen, cyano, C₁-C₆ alkyl, C₂-C₆ alkenyl, C₂-C₆ alkynyl, aryl, alkylaryl, heteroaryl, or alkylheteroaryl, or R₁₂ can combine with X to form a six membered ring, wherein R₁₂ is optionally substituted or unsubstituted;
R₁₃ is hydrogen, halogen, C₁-C₆ alkyl, C₂-C₆ alkenyl, C₂-C₆ alkynyl, cycloalkyl, aryl, alkylaryl, heteroaryl, or alkylheteroaryl, or R₁₃ can combine with D₂ to form a six membered ring, wherein R₁₃ is optionally substituted or unsubstituted; [symbol]
represents a bond that is present or absent;
n is 0 or 1;
or a pharmaceutically acceptable salt, ester, or prodrug thereof.

2. The compound of claim 1, having a structure according to Formula I-A: 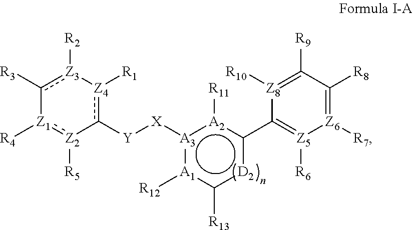
wherein
A₁, A₂, and A₃ are independently C, N or S;
D₂ is O, CR′, NR′ or S wherein R′ is absent or hydrogen, or C₁-C₆ alkyl;
X is absent or O, S, SO, SO₂, CHR′, or NR′, wherein R′ is hydrogen, alkyl, or cycloalkyl;
Y is a bond, —C₁-C₃ alkyl-, —C₂-C₃ alkenyl-, —C₁-C₃ haloalkyl-, —C₂-C₃ haloalkenyl-, —C₁-C₃ alkyloxy-, —C₁-C₃ alkylamine-, —C₁-C₃ alkylamide-, —C₁-C₃ alkylsulfide-, —C₁-C₃ alkylthiol-, —C₁-C₃ alkylsulfoxide-, —C₁-C₃ alkylsulfonyl-, —C₁-C₃ alkylsulfonamide-, —C₁-C₃ ester-, or cyclic, and wherein Y is optionally substituted with one or more groups;
Z₁, Z₂, Z₃, Z₄, Z₅, Z₆, Z₇, and Z₈ are independently C, CH, O, S, or N;
R₁, R₂, R₄, and R₅ are independently absent or hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁, R₂, R₄, and R₅ are optionally substituted with one or more groups;
R₃ is hydrogen, halogen, hydroxyl, amine, C₁-C₆ alkyl, C₂-C₆ alkenyl, C₂-C₆alkynyl, C₁-C₆ haloalkyl, C₂-C₆ haloalkenyl, C₂-C₆ haloalkynyl, C₁-C₆alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₆ to R₁₀ are optionally substituted with one or more groups;
R₁₁ is absent or hydrogen, cyano, C₁-C₆ alkyl, C₂-C₆ alkenyl, C₂-C₆ alkynyl, aryl, alkylaryl, heteroaryl, or alkylheteroaryl, wherein R₁₁ is optionally substituted or unsubstituted;
R₁₂ is absent or hydrogen, cyano, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted; [symbol]
represents a bond that is present or absent;
n is 0 or 1;
or a pharmaceutically acceptable salt, ester, or prodrug thereof.

3. The compound of claim 1, having a structure according to Formula I-A′: 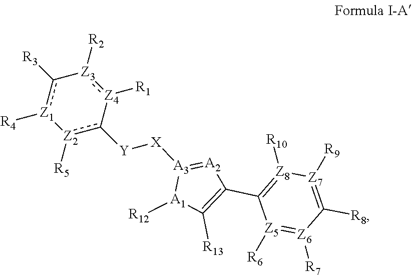
wherein
A₁, A₂, and A₃ are C, N, or S;
X is absent or O, S, SO, SO₂, CHR′, or NR′, wherein R′ is hydrogen, alkyl, or cycloalkyl;
Y is a bond, —C₁-C₃ alkyl-, —C₂-C₃ alkyl-, —C₁-C₃ haloalkyl-, —C₂-C₃ haloalkyl-, —C₁-C₃ alkenyleneoxy-, —C₁-C₃ alkylamine-, —C₁-C₃ alkylamide-, —C₁-C₃ alkylsulfide-, —C₁-C₃ alkylthiol-, —C₁-C₃ alkylsulfoxide-, —C₁-C₃ alkylsulfonyl-, —C₁-C₃ alkylsulfonamide-, —C₁-C₃ ester-, or cyclic, and wherein Y is optionally substituted with one or more groups;
Z₁, Z₂, Z₃, Z₄, Z₅, Z₆, Z₇, and Z₈ are independently C, CH, O, S, or N;
R₁, R₂, R₄, and R₅ are independently absent or hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁, R₂, R₄, and R₅ are optionally substituted with one or more groups;
R₃ is hydrogen, halogen, hydroxyl, amine, C₁-C₆ alkyl, C₂-C₆ alkenyl, C₂-C₆alkynyl, C₁-C₆ haloalkyl, C₂-C₆ haloalkenyl, C₂-C₆ haloalkynyl, C₁-C₆alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₆ to R₁₀ are optionally substituted with one or more groups;
R₁₂ is absent or hydrogen, C₁-C₆ alkyl, C₂-C₆ alkenyl, C₂-C₆ alkynyl, aryl, alkylaryl, heteroaryl, or alkylheteroaryl, wherein R₁₂ is optionally substituted or unsubstituted;
R₁₃ is hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted; [symbol]
represents a bond that is present or absent;
or a pharmaceutically acceptable salt, ester, or prodrug thereof.

4. The compound of claim 1, having a structure according to Formula I-A′-1: 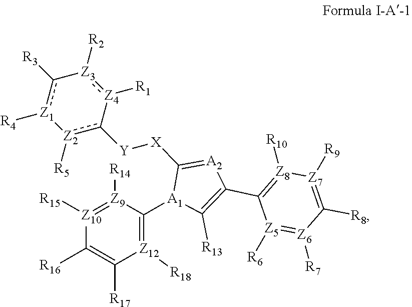
wherein
A₁ and A₂ are independently C, N, or S;
X is absent or O, S, SO, SO₂, CHR′, or NR′, wherein R′ is hydrogen, alkyl, or cycloalkyl;
Y is a bond, —C₁-C₃ alkyl, —C₂-C₃ alkenyl, —C₁-C₃ haloalkyl, —C₂-C₃ haloalkenyl, —C₁-C₃ alkyloxy, —C₁-C₃ alkylamine, —C₁-C₃ alkylamide, —C₁-C₃ alkylsulfide, —C₁-C₃ alkylthiol, —C₁-C₃ alkylsulfoxide, —C₁-C₃ alkylsulfonyl, —C₁-C₃ alkylsulfonamide, —C₁-C₃ ester, or cyclic, and wherein Y is optionally substituted with one or more groups;
Z₁, Z₂, Z₃, Z₄, Z₅, Z₆, Z₇, Z₈, Z₉, Z₁₀, Z₁₁, and Z₁₂ are independently C, CH, O, S, or N;
R₁, R₂, R₄, and R₅ are independently absent or hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁, R₂, R₄, and R₅ are optionally substituted with one or more groups;
R₃ is hydrogen, halogen, hydroxyl, amine, C₁-C₆ alkyl, C₂-C₆ alkenyl, C₂-C₆alkynyl, C₁-C₆ haloalkyl, C₂-C₆ haloalkenyl, C₂-C₆ haloalkynyl, C₁-C₆alkoxy, C₁-C₆ haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently absent or hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₆ to R₁₀ are optionally substituted with one or more groups;
R₁₃ is hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁₄ to R₁₈ are optionally substituted with one or more groups; [symbol]
represents a bond that is present or absent;
or a pharmaceutically acceptable salt, ester, or prodrug thereof.

5. The compound of claim 1, having a structure according to Formula I-A′-2: 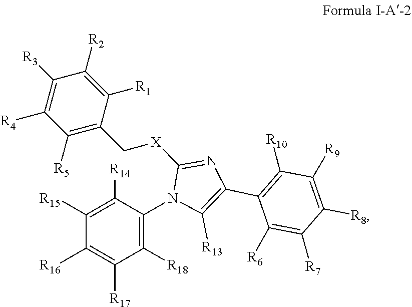
wherein
X is absent or S, SO₂, CHR′, or NR′, wherein R′ is hydrogen, alkyl, or cycloalkyl;
R₁, R₂, R₄, and R₅ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁, R₂, R₄, and R₅ are optionally substituted with one or more groups;
R₃ is hydrogen, halogen, hydroxyl, amine, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆alkoxy, C₁-C₆haloalkoxy, C₁-C₆ alkylamine, —C₁-C₆ alkylsulfide-, —C₁-C₆ alkylthiol-, —C₁-C₆ alkylsulfoxide-, —C₁-C₆ alkylsulfonyl-, —C₁-C₆ alkylsulfonamide-, —C₁-C₆ alkylsulfoximine-, —C₁-C₆ heteroalkyl, cyano, amide, alkylamide, carbamate, alkylcarbamate, thiocarbamate, nitro, aryl, or heteroaryl, wherein R₃ is optionally substituted or unsubstituted;
R₆, R₇, R₈, R₉, and R₁₀ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₆ to R₁₀ are optionally substituted with one or more groups;
R₁₃ is hydrogen, halogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted;
R₁₄, R₁₅, R₁₆, R₁₇, and R₁₈ are independently hydrogen, halogen, alkyl, haloalkyl, heteroalkyl, alkenyl, haloalkenyl, alkynyl, haloalkynyl, alkoxy, haloalkoxy, amine, alkylamine, amide, alkylamide, hydroxyl, cycloalkyl, heterocycloalkyl, cyano, nitro, —R′CO₂H, —CO₂R″, —R′CO₂R″, —CONH₂, —R′CONH₂, —CONHR″, —R′CONHR″, —CONR″R″′, —R′CONR″R″′, —CONHOH, —R′CONHOH, —R′CONHCN, —NR′C(═O)—R″, —SR″, —SO—R″, —SO₂—R″, —R′SO₃R″, —R′SO₂NHCOR″, —R′CONHSO₂R″, —SO₂NR′R″, —R′SO₂NR′R″, —NR′SO₂R″, —OCONR′R″, —NR′CO₂—R″, —OCO₂—R″, —NHCONH—R″, —OCO—R″, —NR′R″, cycloalkyl, cycloalkenyl, heterocycloalkyl, heteroalkyl, cycloheteroalkenyl, aryl, or heteroaryl, wherein R′, R″, and R″′ are independently hydrogen, C₁-C₆ alkyl, C₂-C₆ alkenyl, C₂-C₆ alkynyl, C₁-C₆ alkoxy, C₁-C₆ haloalkyl, cycloalkyl, alkyl cycloalkyl, cycloalkenyl, alkyl cycloalkenyl, heterocycloalkyl, alkyl heterocycloalkyl, cycloheteroalkenyl, aryl, alkylaryl, heteroaryl, or alkylheteroaryl, and wherein R₁₄ to R₁₈ are optionally substituted with one or more groups;
or a pharmaceutically acceptable salt, ester, or prodrug thereof.

6. The compound of claim 1, wherein X is absent or O, S, SO, SO₂, or NR′, wherein R′ is hydrogen or alkyl.

7. The compound of claim 1, wherein X is S, O or NH and Y is —C₁-C₃ alkyl.

8. The compound of claim 1, wherein R₃ is hydrogen, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₁-C₆ alkoxy, C₁-C₆ haloalkoxy, C₁-C₆ alkylamine, —C₁-C₆ alkylsulfoxide-, —C₁-C₆ alkylsulfonyl-, —C₁-C₆ alkylsulfonamide-, cyano, amide, alkylamide, carbamate, or alkylcarbamate, wherein R₃ is optionally substituted or unsubstituted.

9. The compound of claim 1, wherein R₁₃ is hydrogen, C₁-C₆ alkyl, C₂-C₆ alkenyl, C₂-C₆ alkynyl, cycloalkyl, aryl, alkylaryl, heteroaryl, or alkylheteroaryl, wherein R₁₃ is optionally substituted or unsubstituted.

10. The compound of claim 1, wherein R₁₁ and R₁₃ are hydrogen, aryl, alkylaryl, heteroaryl, or alkylheteroaryl, wherein R₁₃ is optionally substituted or unsubstituted.

11. The compound of claim 1, wherein R₁₂ is C₂-C₆ alkenyl, C₂-C₆ alkynyl, aryl, alkylaryl, heteroaryl, or alkylheteroaryl, wherein R₁₂ is optionally substituted or unsubstituted.

12. The compound of claim 1, wherein the compound is selected from the group consisting of: 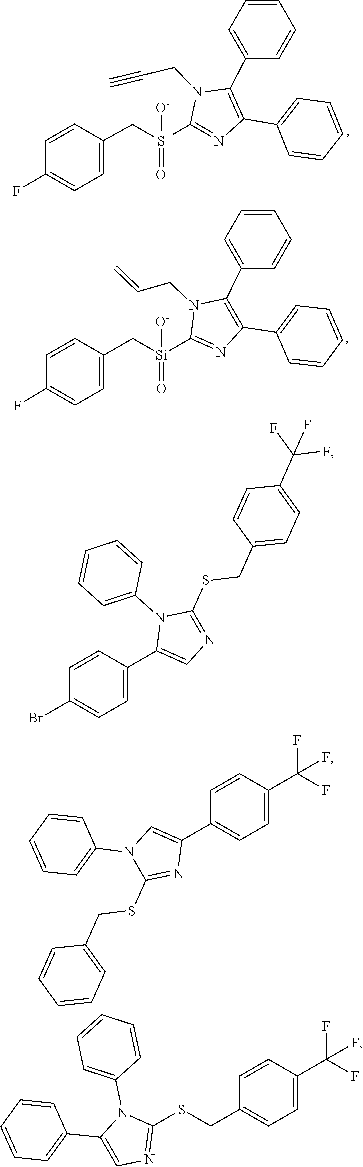 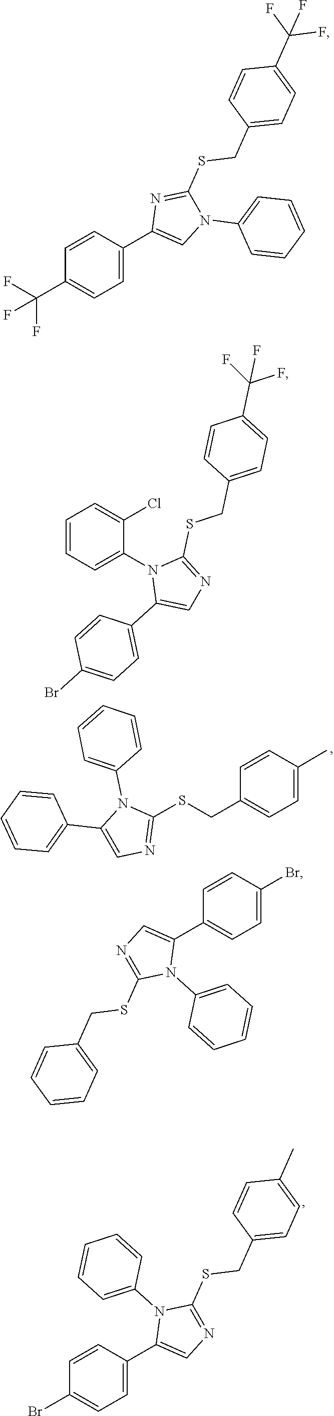 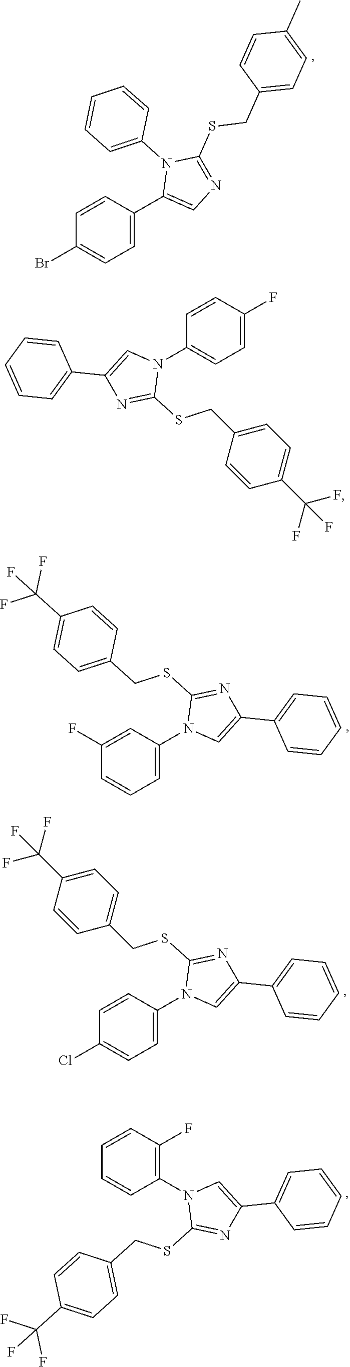 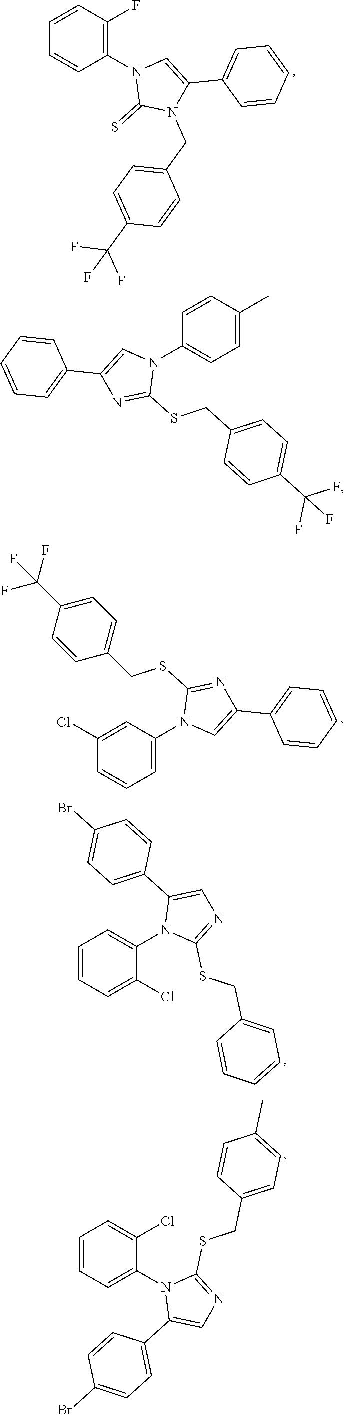 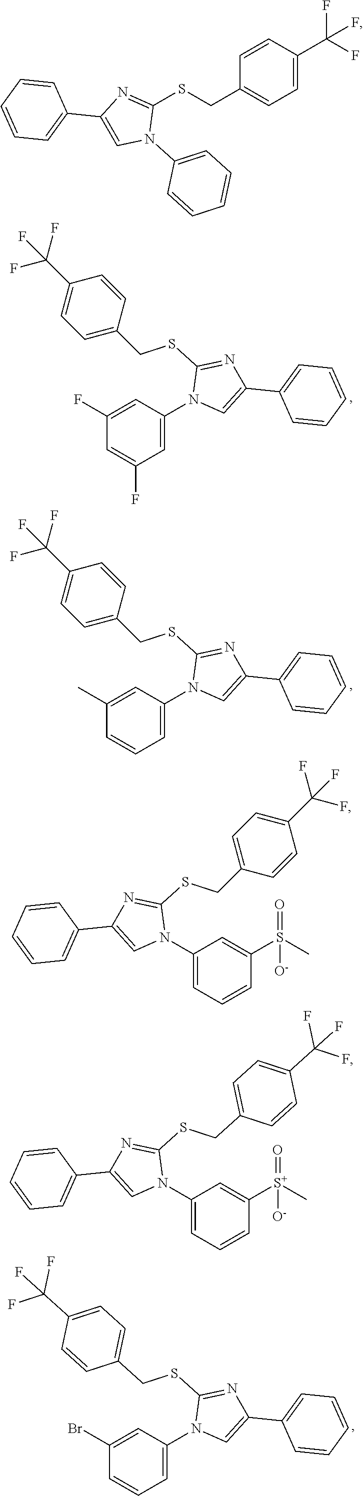 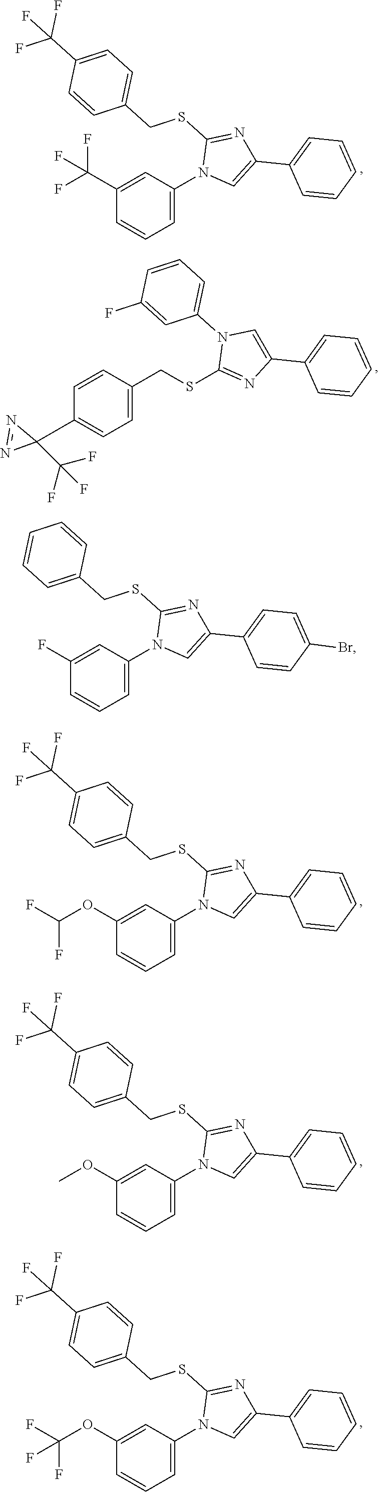 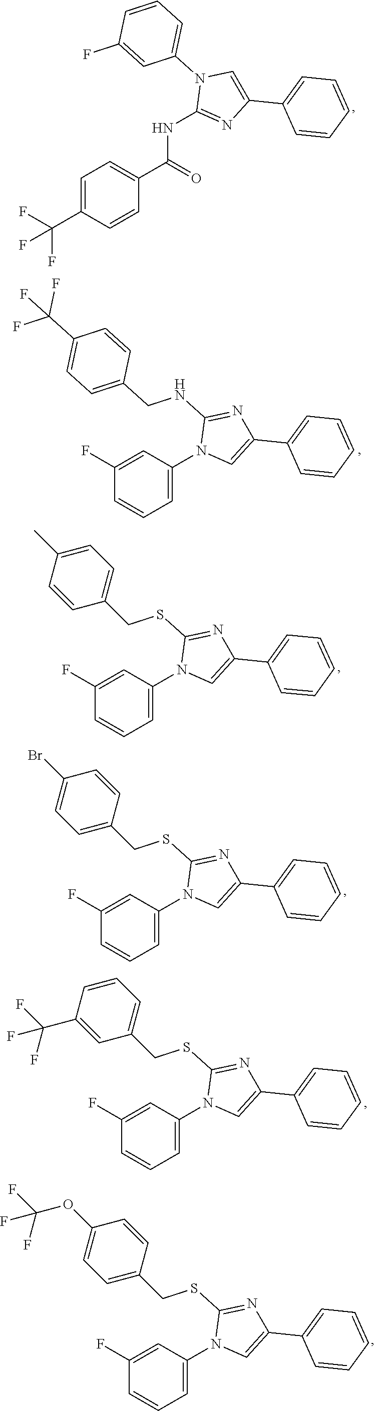 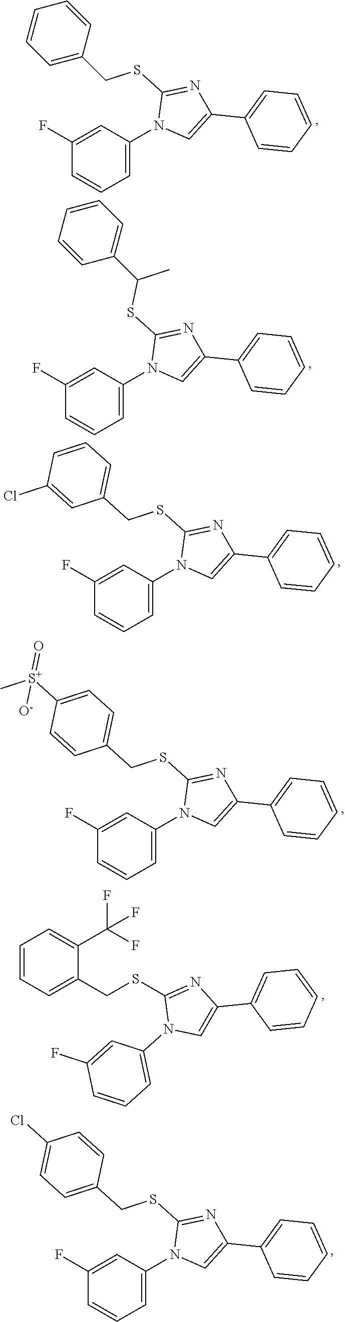 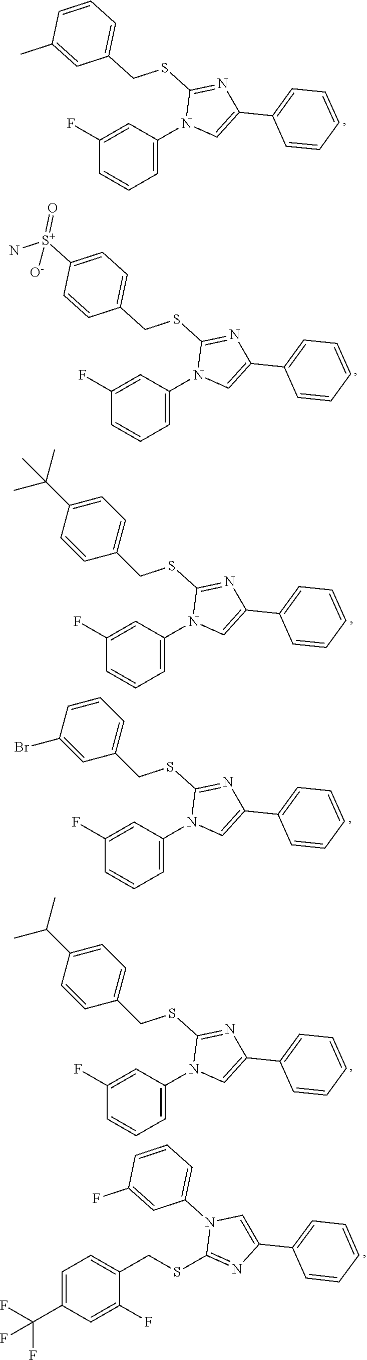 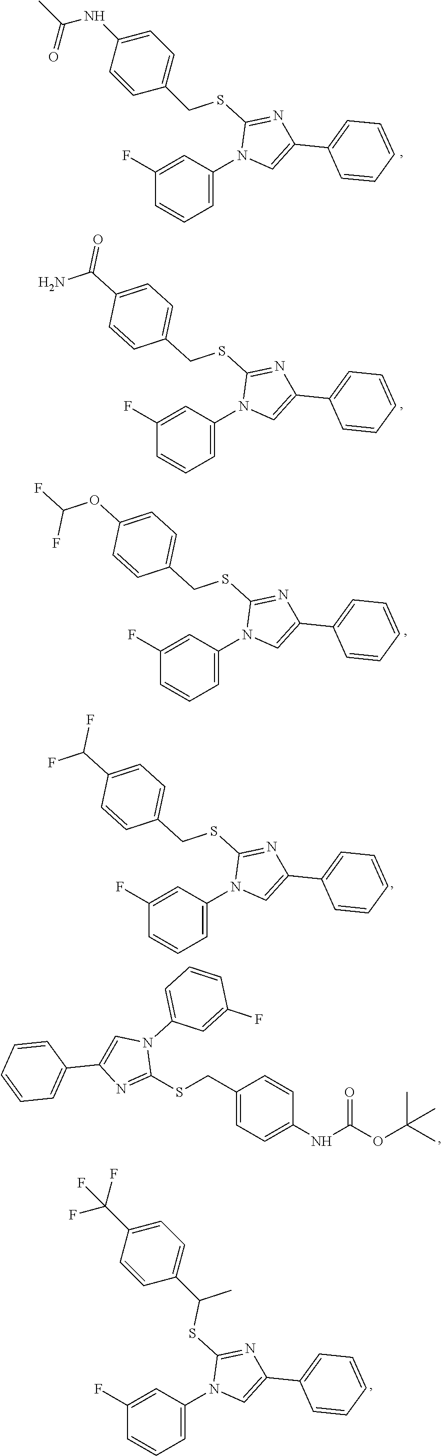 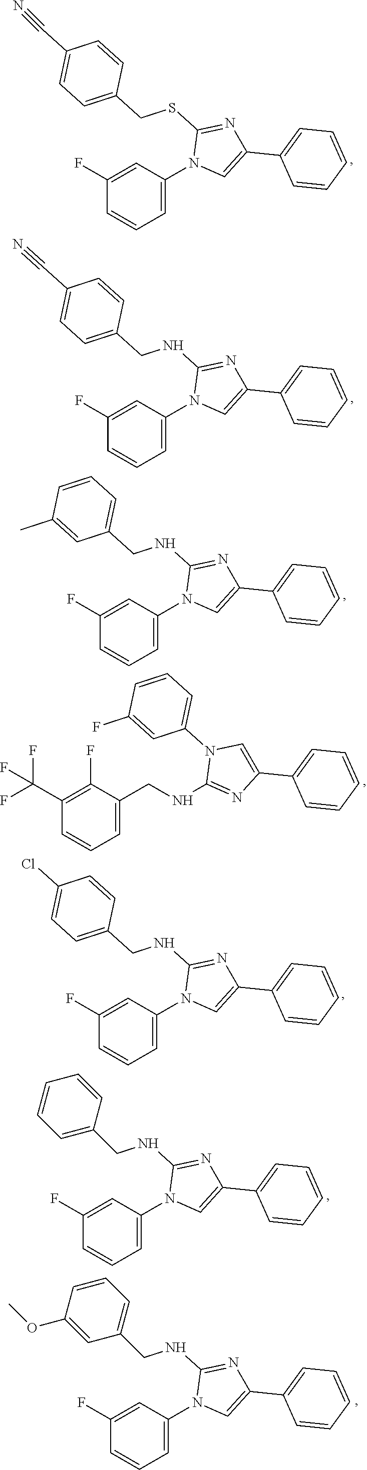 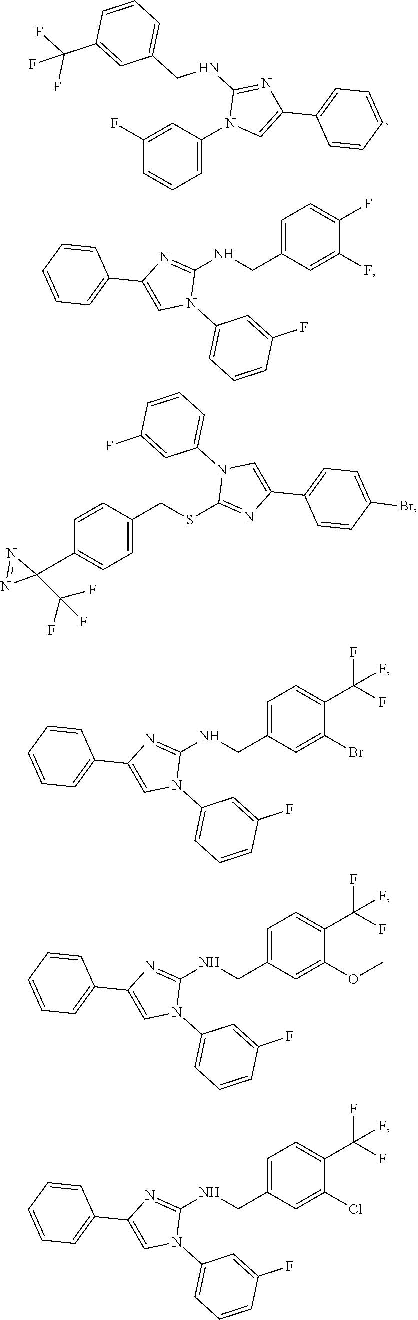 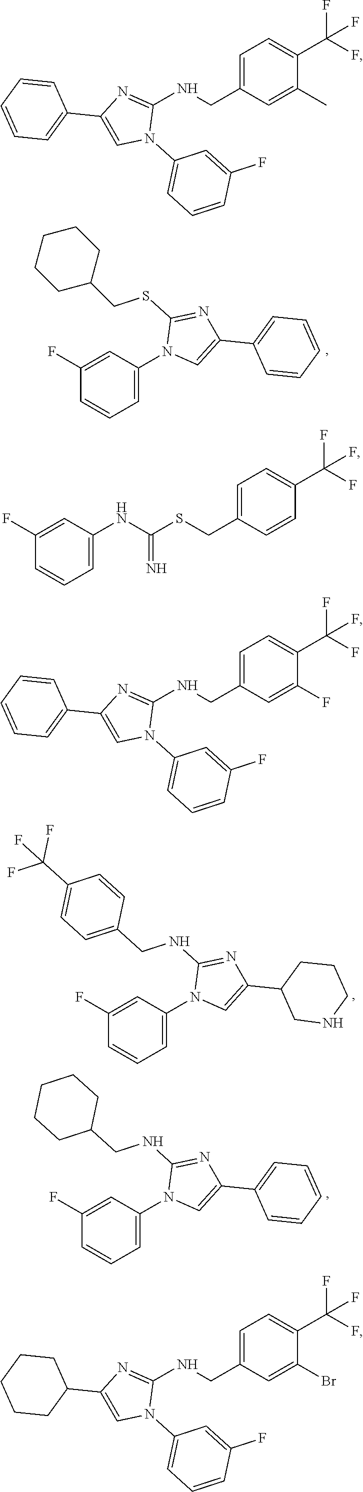 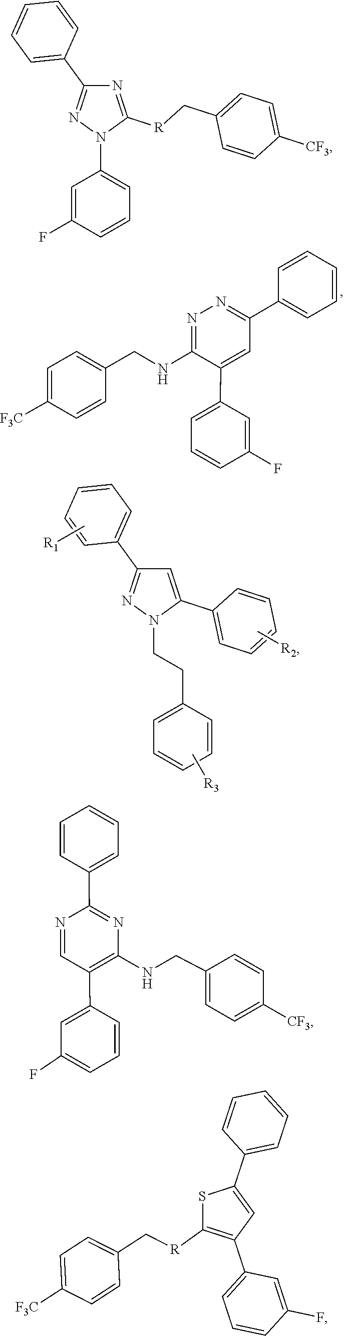 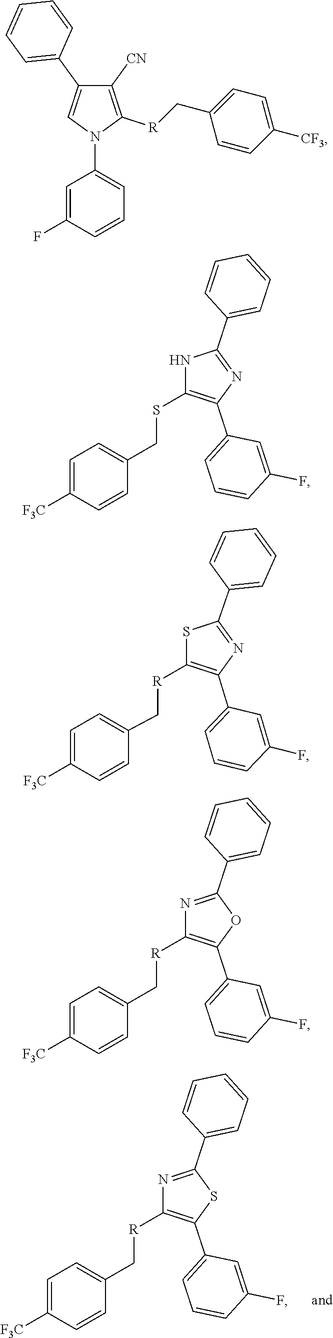 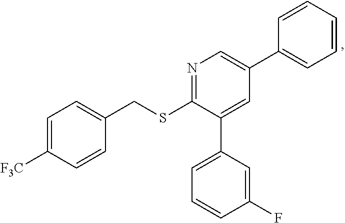
wherein R is S or NH; a pharmaceutically acceptable salt, ester, or prodrug thereof; and combinations thereof.

13. The compound of claim 1, wherein the compound is selected from the group consisting of: 2-((4-fluorobenzyl)sulfonyl)-4,5-diphenyl-1-(prop-2-yn-1-yl)-1H-imidazole; 1-allyl-2-((4-fluorobenzyl)sulfonyl)-4,5-diphenyl-1H-imidazole; 5-(4-bromophenyl)-1-phenyl-2-((4-(trifluoromethyl)benzyl)thio)-1H-imidazole; 2-(benzylthio)-1-phenyl-4-(4-(trifluoromethyl)phenyl)-1H-imidazole; 1,5-diphenyl-2-((4-(trifluoromethyl)benzyl)thio)-1H-imidazole; 1-phenyl-2-((4-(trifluoromethyl)benzyl)thio)-4-(4-(trifluoromethyl)phenyl)-1H-imidazole; 5-(4-bromophenyl)-1-(2-chlorophenyl)-2-((4-(trifluoromethyl)benzyl)thio)-1H-imidazole; 2-((4-methylbenzyl)thio)-1,5-diphenyl-1H-imidazole; 2-(benzylthio)-5-(4-bromophenyl)-1-phenyl-1H-imidazole; 5-(4-bromophenyl)-2-((4-methylbenzyl)thio)-1-phenyl-1H-imidazole; 1-(4-fluorophenyl)-4-phenyl-2-((4-(trifluoromethyl)benzyl)thio)-1H-imidazole; 1-(3-fluorophenyl)-4-phenyl-2-((4-(trifluoromethyl)benzyl)thio)-1H-imidazole; 1-(4-chlorophenyl)-4-phenyl-2-((4-(trifluoromethyl)benzyl)thio)-1H-imidazole; 1-(2-fluorophenyl)-4-phenyl-2-((4-(trifluoromethyl)benzyl)thio)-1H-imidazole; 1-(2-fluorophenyl)-4-phenyl-3-(4-(trifluoromethyl)benzyl)-1,3-dihydro-2H-imidazole-2-thione; 4-phenyl-1-(p-tolyl)-2-((4-(trifluoromethyl)benzyl)thio)-1H-imidazole; 1-(3-chlorophenyl)-4-phenyl-2-((4-(trifluoromethyl)benzyl)thio)-1H-imidazole; 2-(benzylthio)-5-(4-bromophenyl)-1-(2-chlorophenyl)-1H-imidazole; 5-(4-bromophenyl)-1-(2-chlorophenyl)-2-((4-methylbenzyl)thio)-1H-imidazole; 1,4-diphenyl-2-((4-(trifluoromethyl)benzyl)thio)-1H-imidazole; 1-(3,5-difluorophenyl)-4-phenyl-2-((4-(trifluoromethyl)benzyl)thio)-1H-imidazole; 4-phenyl-1-(m-tolyl)-2-((4-(trifluoromethyl)benzyl)thio)-1H-imidazole; 1-(3-(methylsulfonyl)phenyl)-4-phenyl-2-((4-(trifluoromethyl)benzyl)thio)-1H-imidazole; 1-(3-bromophenyl)-4-phenyl-2-((4-(trifluoromethyl)benzyl)thio)-1H-imidazole; 4-phenyl-2-((4-(trifluoromethyl)benzyl)thio)-1-(3-(trifluoromethyl)phenyl)-1H-imidazole; 1-(3-fluorophenyl)-4-phenyl-2-((4-(3-(trifluoromethyl)-3H-diazirin-3-yl)benzyl)thio)-1H-imidazole; 2-(benzylthio)-4-(4-bromophenyl)-1-(3-fluorophenyl)-1H-imidazole; 1-(3-(difluoromethoxy)phenyl)-4-phenyl-2-((4-(trifluoromethyl)benzyl)thio)-1H-imidazole; 1-(3-methoxyphenyl)-4-phenyl-2-((4-(trifluoromethyl)benzyl)thio)-1H-imidazole; 4-phenyl-1-(3-(trifluoromethoxy)phenyl)-2-((4-(trifluoromethyl)benzyl)thio)-1H-imidazole; N-(1-(3-fluorophenyl)-4-phenyl-1H-imidazol-2-yl)-4-(trifluoromethyl)benzamide; 1-(3-fluorophenyl)-4-phenyl-N-(4-(trifluoromethyl)benzyl)-1H-imidazol-2-amine; 1-(3-fluorophenyl)-2-((4-methylbenzyl)thio)-4-phenyl-1H-imidazole; 2-((4-bromobenzyl)thio)-1-(3-fluorophenyl)-4-phenyl-1H-imidazole; 1-(3-fluorophenyl)-4-phenyl-2-((3-(trifluoromethyl)benzyl)thio)-1H-imidazole; 1-(3-fluorophenyl)-4-phenyl-2-((4-(trifluoromethoxy)benzyl)thio)-1H-imidazole; 2-(benzylthio)-1-(3-fluorophenyl)-4-phenyl-1H-imidazole; 1-(3-fluorophenyl)-4-phenyl-2-((1-phenylethyl)thio)-1H-imidazole; 2-((3-chlorobenzyl)thio)-1-(3-fluorophenyl)-4-phenyl-1H-imidazole; 1-(3-fluorophenyl)-2-((4-(methylsulfonyl)benzyl)thio)-4-phenyl-1H-imidazole; 1-(3-fluorophenyl)-4-phenyl-2-((2-(trifluoromethyl)benzyl)thio)-1H-imidazole; 2-((4-chlorobenzyl)thio)-1-(3-fluorophenyl)-4-phenyl-1H-imidazole; 1-(3-fluorophenyl)-2-((3-methylbenzyl)thio)-4-phenyl-1H-imidazole; 4-(((1-(3-fluorophenyl)-4-phenyl-1H-imidazol-2-yl)thio)methyl)benzenesulfonamide; 2-((4-(tert-butyl)benzyl)thio)-1-(3-fluorophenyl)-4-phenyl-1H-imidazole; 2-((3-bromobenzyl)thio)-1-(3-fluorophenyl)-4-phenyl-1H-imidazole; 1-(3-fluorophenyl)-2-((4-isopropylbenzyl)thio)-4-phenyl-1H-imidazole; 2-((2-fluoro-4-(trifluoromethyl)benzyl)thio)-1-(3-fluorophenyl)-4-phenyl-1H-imidazole; N-(4-(((1-(3-fluorophenyl)-4-phenyl-1H-imidazol-2-yl)thio)methyl)phenyl)acetamide; 4-(((1-(3-fluorophenyl)-4-phenyl-1H-imidazol-2-yl)thio)methyl)benzamide; 2-((4-(difluoromethoxy)benzyl)thio)-1-(3-fluorophenyl)-4-phenyl-1H-imidazole; 2-((4-(difluoromethyl)benzyl)thio)-1-(3-fluorophenyl)-4-phenyl-1H-imidazole; 1 tert-butyl (4-(((1-(3-fluorophenyl)-4-phenyl-1H-imidazol-2-yl)thio)methyl)phenyl)carbamate; 1-(3-fluorophenyl)-4-phenyl-2-((1-(4-(trifluoromethyl)phenyl)ethyl)thio)-1H-imidazole; 4-(((1-(3-fluorophenyl)-4-phenyl-1H-imidazol-2-yl)thio)methyl)benzonitrile; 4-(((1-(3-fluorophenyl)-4-phenyl-1H-imidazol-2-yl)amino)methyl)benzonitrile; 1-(3-fluorophenyl)-N-(3-methylbenzyl)-4-phenyl-1H-imidazol-2-amine; N-(2-fluoro-3-(trifluoromethyl)benzyl)-1-(3-fluorophenyl)-4-phenyl-1H-imidazol-2-amine; N-(4-chlorobenzyl)-1-(3-fluorophenyl)-4-phenyl-1H-imidazol-2-amine; N-(4-chlorobenzyl)-1-(3-fluorophenyl)-4-phenyl-1H-imidazol-2-amine; N-benzyl-1-(3-fluorophenyl)-4-phenyl-1H-imidazol-2-amine; 1-(3-fluorophenyl)-N-(3-methoxybenzyl)-4-phenyl-1H-imidazol-2-amine; 1-(3-fluorophenyl)-4-phenyl-N-(3-(trifluoromethyl)benzyl)-1H-imidazol-2-amine; N-(3,4-difluorobenzyl)-1-(3-fluorophenyl)-4-phenyl-1H-imidazol-2-amine; 4-(4-bromophenyl)-1-(3-fluorophenyl)-2-((4-(3-(trifluoromethyl)-3H-diazirin-3-yl)benzyl)thio)-1H-imidazole; N-(3-bromo-4-(trifluoromethyl)benzyl)-1-(3-fluorophenyl)-4-phenyl-1H-imidazol-2-amine; 1-(3-fluorophenyl)-N-(3-methoxy-4-(trifluoromethyl)benzyl)-4-phenyl-1H-imidazol-2-amine; N-(3-chloro-4-(trifluoromethyl)benzyl)-1-(3-fluorophenyl)-4-phenyl-1H-imidazol-2-amine; 1-(3-fluorophenyl)-N-(3-methyl-4-(trifluoromethyl)benzyl)-4-phenyl-1H-imidazol-2-amine; 2-((cyclohexylmethyl)thio)-1-(3-fluorophenyl)-4-phenyl-1H-imidazole; 4-(trifluoromethyl)benzyl (3-fluorophenyl)carbamimidothioate; N-(3-fluoro-4-(trifluoromethyl)benzyl)-1-(3-fluorophenyl)-4-phenyl-1H-imidazol-2-amine; 1-(3-fluorophenyl)-4-(piperidin-3-yl)-N-(4-(trifluoromethyl)benzyl)-1H-imidazol-2-amine; N-(cyclohexylmethyl)-1-(3-fluorophenyl)-4-phenyl-1H-imidazol-2-amine; N-(3-bromo-4-(trifluoromethyl)benzyl)-4-cyclohexyl-1-(3-fluorophenyl)-1H-imidazol-2-amine; 4-(1-(3-fluorophenyl)-2-((4-(trifluoromethyl)benzyl)amino)-1H-imidazol-4-yl)benzonitrile; N-(3-bromo-4-(trifluoromethyl)benzyl)-4-(4-ethynylphenyl)-1-(3-fluorophenyl)-1H-imidazol-2-amine; 4-(4-ethynylphenyl)-1-(3-fluorophenyl)-N-(4-(trifluoromethyl)benzyl)-1H-imidazol-2-amine; 4-(4-ethoxyphenyl)-1-(3-fluorophenyl)-N-(4-(trifluoromethyl)benzyl)-1H-imidazol-2-amine; 1-(3-fluorophenyl)-N-(4-(trifluoromethyl)benzyl)-4-(3-(trifluoromethyl)phenyl)-1H-imidazol-2-amine; N-(3-bromo-4-(trifluoromethyl)benzyl)-4-(4-ethoxyphenyl)-1-(3-fluorophenyl)-1H-imidazol-2-amine; 4-(4-ethoxyphenyl)-1-(3-fluorophenyl)-N-(3-methoxy-4-(trifluoromethyl)benzyl)-1H-imidazol-2-amine; 3,5-diphenyl-1-(4-(trifluoromethyl)benzyl)-1H-pyrazole; 1-(3-fluorophenyl)-4-(tetrahydro-2H-pyran-4-yl)-N-(4-(trifluoromethyl)benzyl)-1H-imidazol-2-amine; 4-(4-bromophenyl)-N-(3-fluoro-4-(trifluoromethyl)benzyl)-1-(3-fluorophenyl)-1H-imidazol-2-amine; 1-phenethyl-3,5-diphenyl-1H-pyrazole; 4-(4-bromophenyl)-1-(3-fluorophenyl)-N-(4-(trifluoromethyl)benzyl)-1H-imidazol-2-amine; 1-(3-fluorophenyl)-4-phenyl-N-((4-(trifluoromethyl)phenyl)methyl-d2)-1H-imidazol-2-amine; 4-(4-bromophenyl)-1-(3-fluorophenyl)-N-(3-methoxy-4-(trifluoromethyl)benzyl)-1H-imidazol-2-amine; 1-(3-fluorophenyl)-N-(3-methoxy-4-(trifluoromethyl)benzyl)-4-(tetrahydro-2H-pyran-4-yl)-1H-imidazol-2-amine; N-(3-fluoro-4-(trifluoromethyl)benzyl)-1-(3-fluorophenyl)-4-(tetrahydro-2H-pyran-4-yl)-1H-imidazol-2-amine; N-(3-bromo-4-(trifluoromethyl)benzyl)-1-(3-fluorophenyl)-4-(tetrahydro-2H-pyran-4-yl)-1H-imidazol-2-amine; 4-(4-chlorophenyl)-1-(3-fluorophenyl)-N-(4-(trifluoromethyl)benzyl)-1H-imidazol-2-amine; 4-(4-ethynylphenyl)-1-(3-fluorophenyl)-N-(3-methoxy-4-(trifluoromethyl)benzyl)-1H-imidazol-2-amine; 1-phenyl-N-(4-(trifluoromethyl)phenethyl)-1H-pyrazol-4-amine; 1-phenyl-N-(4-(trifluoromethyl)phenethyl)-1H-pyrazol-3-amine; 1-(3-bromophenyl)-N-(3-methoxy-4-(trifluoromethyl)benzyl)-4-phenyl-1H-imidazol-2-amine; N-(3-methoxy-4-(trifluoromethyl)benzyl)-4-phenyl-1-(m-tolyl)-1H-imidazol-2-amine; 1,4-diphenyl-N-(4-(trifluoromethyl)benzyl)-1H-pyrazol-3-amine; 4-(3-fluorophenyl)-1-phenyl-N-(4-(trifluoromethyl)benzyl)-1H-pyrazol-3-amine; 3,5-diphenyl-1-(4-(trifluoromethyl)phenethyl)-1H-pyrazole; 4-(3,5-difluorophenyl)-1-phenyl-N-(4-(trifluoromethyl)benzyl)-1H-pyrazol-3-amine; 5-(3-fluorophenyl)-1-phenyl-N-(4-(trifluoromethyl)phenethyl)-1H-pyrazol-4-amine; 1,5-diphenyl-3-((4-(trifluoromethyl)benzyl)thio)-1H-1,2,4-triazole; 1,3-diphenyl-5-((4-(trifluoromethyl)benzyl)thio)-1H-1,2,4-triazole; a pharmaceutically acceptable salt, ester, or prodrug thereof; and combinations thereof.

14. A pharmaceutical composition comprising a therapeutic effective amount of a compound according to claim 1, and a pharmaceutically acceptable carrier.

15. A method of preventing or treating necroinflammation associated with ferroptotic processes promoting upper or lower respiratory disorders, acute or chronic brain injury, renal injury, injury by radiation, neurodegenerative disorder, or a combination thereof in a subject in need thereof, the method comprising:
administering a therapeutically effective amount of a compound or pharmaceutical composition that inhibits 15 lipoxygenase/phosphatidylethanolamine binding protein (15LOX/PEBP1) complex and/or inhibits interaction of 15 lipoxygenase (15LOX) with phosphatidylethanolamine binding protein PEBP1 to the subject,
wherein the compound or pharmaceutical composition exhibits a higher binding affinity or binding activity for 15LOX/PEBP1 complex compared to 15LOX alone.

16-18. (canceled)

19. A method of preventing or treating necroinflammation associated with ferroptotic processes thereby promoting upper or lower respiratory disorders, acute or chronic brain injury, renal injury, injury by radiation, neurodegenerative disorder, or a combination thereof in a subject in need thereof,
the method comprising administering a therapeutic effective amount of a compound having a structure according to claim 1 to the subject,
wherein the compound or composition inhibits accumulation of 15-hydroperoxy-eicasotetraenoyl-phosphatidylethanolamines (15 HpETE-PE) in the subject.

20-32. (canceled)

33. A method of preventing or treating necroinflammation associated with ferroptotic processes, comprising administering to a subject in need thereof a therapeutically effective amount of a compound according to claim 1.

34. The compound of claim 1, wherein X is O, S, SO, SO₂, CHR′, or NR′, wherein R′ is hydrogen, alkyl, or cycloalkyl.

35. The compound of claim 1, wherein X is O, S, SO, SO₂, or NR′, wherein R′ is hydrogen or alkyl.

36. The compound of claim 1, wherein X is S, O or NH.
